SEQUENCE LISTING (31 sequences)

SEQ ID NO: 1 - protein, 10 aa, synthetic construct
  REGION 1..10: note = VH-CDR1
GYTFTSYNMH

SEQ ID NO: 2 - protein, 17 aa, synthetic construct
  REGION 1..17: note = VH-CDR2
GIYPGNGDTSYNQKFQG

SEQ ID NO: 3 - protein, 11 aa, synthetic construct
  REGION 1..11: note = VH-CDR3
GTVVGDWYFDV

SEQ ID NO: 4 - protein, 16 aa, synthetic construct
  REGION 1..16: note = VL-CDR1
RSSQSLENSNGNTYLN

SEQ ID NO: 5 - protein, 7 aa, synthetic construct
  REGION 1..7: note = VL-CDR2
RVSNRFS

SEQ ID NO: 6 - protein, 9 aa, synthetic construct
  REGION 1..9: note = VL-CDR3
LQLTHVPWT

SEQ ID NO: 7 - protein, 120 aa, synthetic construct
  REGION 1..120: note = Heavy chain variable region
QVQLVQSGAEVKKPGASVKVSCKASGYTFTSYNMHWVRQAPGQGLEWMGGIYPGNGDTSYNQKFQGRVTMTRDTSTSTVYMELSSLRSEDTAVYYCARGTVVGDWYFDVWGQGTLVTVSS

SEQ ID NO: 8 - protein, 113 aa, synthetic construct
  REGION 1..113: note = Light chain variable region
DIQMTQSPSSLSASVGDRVTITCRSSQSLENSNGNTYLNWYQQKPGKAPKLLIYRVSNRFSGVPSRFSGSGSGTDFTFTISSLQPEDIATYYCLQLTHVPWTFGQGTKVEITR

SEQ ID NO: 9 - protein, 113 aa, synthetic construct
  REGION 1..113: note = Light chain variable region
DIQMTQSPSSLSASVGDRVTITCSASQSLENSNGNTYLNWYQQKPGKAPKLLIYRVSNRFSGVPSRFSGSGSGTDFTLTISSLQPEDFATYYCLQLTHVPWTFGQGTKVEIKR

SEQ ID NO: 10 - DNA, 360 bp, synthetic construct
  misc_feature 1..360: note = Heavy chain variable region
  source 1..360: mol_type = other DNA
caggttcagctggttcagtcaggggctgaggtgaagaagcctggagcctcagtgaaggtgtcctgcaaggcttctggctacacatttaccagttacaatatgcactgggtaagacaggctcctggacagggcctggaatggatgggaggtatttatccaggaaatggtgatacttcctacaatcagaagttccagggccgagttactatgactcgggacacttccacctctacagtgtacatggagctcagcagcctgagatctgaggacaccgcggtctattactgtgcaagaggcaccgtggtcggcgactggtacttcgatgtgtggggccaaggcaccctggtcaccgtgagcagt

SEQ ID NO: 11 - DNA, 339 bp, synthetic construct
  misc_feature 1..339: note = Light chain variable region
  source 1..339: mol_type = other DNA
gacatccagatgacccagagcccaagcagcctgagcgccagcgtgggtgacagagtgaccatcacctgtagaagtagtcagagccttgagaacagcaacggaaacacctacctgaattggtaccagcagaagccaggtaaggctccaaagctgctgatctacagagtttccaaccgattctctggtgtgccaagcagattcagcggtagcggtagcggtaccgacttcaccttcaccatcagcagcctccagccagaggacatcgccacctactactgcctccagctgactcatgtcccatggaccttcggtcagggcaccaaggtggagatcacccgg

SEQ ID NO: 12 - DNA, 339 bp, synthetic construct
  misc_feature 1..339: note = Light chain variable region
  source 1..339: mol_type = other DNA
gacatccagatgacccagagcccaagcagcctgagcgccagcgtgggtgacagagtgaccatcacctgtagtgccagtcagagccttgagaacagcaacggaaacacctacctgaattggtaccagcagaagccaggtaaggctccaaagctgctgatctacagagtttccaaccgattctctggtgtgccaagcagattcagcggtagcggtagcggtaccgacttcaccctcaccatcagcagcctccagccagaggacttcgccacctactactgcctccagctgactcatgtcccatggaccttcggtcagggcaccaaggtggagatcaaacgc

SEQ ID NO: 13 - protein, 339 aa, Homo sapiens
MSPSPTALFCLGLCLGRVPAQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGNLVRICLGAVILIILAGFLAEDWHSRRKRLRHRGRAVQRPLPPLPPLPQTRKSHGGQDGGRQDVHSRGLCS

SEQ ID NO: 14 - DNA, 321 bp, synthetic construct
  misc_feature 1..321: note = Heavy chain constant region
  source 1..321: mol_type = other DNA
gcctccaccaagggtccctcagtcttcccactggcaccctcctccaagagcacctctggtggcacagctgccctgggctgcctggtcaaggactacttcccagaaccagtgactgtgtcatggaactcaggcgccctgaccagcggcgtgcacaccttccctgctgtcttgcagtcctcaggactctactccctcagcagcgtggtgaccgtgccctccagcagcttgggcacccagacctacatctgcaacgtgaatcacaagcccagcaacaccaaggtggacaagaaagtcgagcctaagtcatgcgacaagactcac

SEQ ID NO: 15 - DNA, 318 bp, synthetic construct
  misc_feature 1..318: note = Light chain constant region
  source 1..318: mol_type = other DNA
actgtggctgcaccaagtgtgttcatcttcccacctagcgatgagcagttgaaatctggaactgcctctgtcgtgtgcctcctgaacaacttctacccacgggaggccaaggtacagtggaaggtggataacgccctccaatccggtaactcccaggagagtgtcacagagcaagatagcaaggacagcacctacagcctcagcagcaccctgactctgagcaaagcagactacgagaagcacaaggtctacgcctgcgaagtcacccatcagggcctgagttcccctgtcacaaagagcttcaaccggggagagtgt

SEQ ID NO: 16 - DNA, 66 bp, synthetic construct
  misc_feature 1..66: note = Signal peptide
  source 1..66: mol_type = other DNA
atggatatgcgtgtaccagctcaactacttggacttctattgctttggcttcgtggtgctagatgt

SEQ ID NO: 17 - DNA, 57 bp, synthetic construct
  misc_feature 1..57: note = Signal peptide
  source 1..57: mol_type = other DNA
atggactggacttggagaatcctattcttggttgctgcagctacaggtgctcattca

SEQ ID NO: 18 - DNA, 762 bp, synthetic construct
  misc_feature 1..762: note = Heavy chain
  source 1..762: mol_type = other DNA
gcggccgccaccatggactggacttggagaatcctattcttggttgctgcagctacaggtgctcattcacaggttcagctggttcagtcaggggctgaggtgaagaagcctggagcctcagtgaaggtgtcctgcaaggcttctggctacacatttaccagttacaatatgcactgggtaagacaggctcctggacagggcctggaatggatgggaggtatttatccaggaaatggtgatacttcctacaatcagaagttccagggccgagttactatgactcgggacacttccacctctacagtgtacatggagctcagcagcctgagatctgaggacaccgcggtctattactgtgcaagaggcaccgtggtcggcgactggtacttcgatgtgtggggccaaggcaccctggtcaccgtgagcagtgcctccaccaagggtccctcagtcttcccactggcaccctcctccaagagcacctctggtggcacagctgccctgggctgcctggtcaaggactacttcccagaaccagtgactgtgtcatggaactcaggcgccctgaccagcggcgtgcacaccttccctgctgtcttgcagtcctcaggactctactccctcagcagcgtggtgaccgtgccctccagcagcttgggcacccagacctacatctgcaacgtgaatcacaagcccagcaacaccaaggtggacaagaaagtcgagcctaagtcatgcgacaagactcactgatgaggatcc

SEQ ID NO: 19 - DNA, 742 bp, synthetic construct
  misc_feature 1..742: note = Light chain
  source 1..742: mol_type = other DNA
gacgtcaccatggatatgcgtgtaccagctcaactacttggacttctattgctttggcttcgtggtgctagatgtgacatccagatgacccagagcccaagcagcctgagcgccagcgtgggtgacagagtgaccatcacctgtagaagtagtcagagccttgagaacagcaacggaaacacctacctgaattggtaccagcagaagccaggtaaggctccaaagctgctgatctacagagtttccaaccgattctctggtgtgccaagcagattcagcggtagcggtagcggtaccgacttcaccttcaccatcagcagcctccagccagaggacatcgccacctactactgcctccagctgactcatgtcccatggaccttcggtcagggcaccaaggtggagatcacccggactgtggctgcaccaagtgtgttcatcttcccacctagcgatgagcagttgaaatctggaactgcctctgtcgtgtgcctcctgaacaacttctacccacgggaggccaaggtacagtggaaggtggataacgccctccaatccggtaactcccaggagagtgtcacagagcaagatagcaaggacagcacctacagcctcagcagcaccctgactctgagcaaagcagactacgagaagcacaaggtctacgcctgcgaagtcacccatcagggcctgagttcccctgtcacaaagagcttcaaccggggagagtgttgatgatatc

SEQ ID NO: 20 - DNA, 742 bp, synthetic construct
  misc_feature 1..742: note = Light chain
  source 1..742: mol_type = other DNA
gacgtcaccatggatatgcgtgtaccagctcaactacttggacttctattgctttggcttcgtggtgctagatgtgacatccagatgacccagagcccaagcagcctgagcgccagcgtgggtgacagagtgaccatcacctgtagtgccagtcagagccttgagaacagcaacggaaacacctacctgaattggtaccagcagaagccaggtaaggctccaaagctgctgatctacagagtttccaaccgattctctggtgtgccaagcagattcagcggtagcggtagcggtaccgacttcaccctcaccatcagcagcctccagccagaggacttcgccacctactactgcctccagctgactcatgtcccatggaccttcggtcagggcaccaaggtggagatcaaacgcactgtggctgcaccaagtgtgttcatcttcccacctagcgatgagcagttgaaatctggaactgcctctgtcgtgtgcctcctgaacaacttctacccacgggaggccaaggtacagtggaaggtggataacgccctccaatccggtaactcccaggagagtgtcacagagcaagatagcaaggacagcacctacagcctcagcagcaccctgactctgagcaaagcagactacgagaagcacaaggtctacgcctgcgaagtcacccatcagggcctgagttcccctgtcacaaagagcttcaaccggggagagtgttgatgatatc

SEQ ID NO: 21-22 - skipped (2 entries)

SEQ ID NO: 23 - protein, 5 aa, synthetic construct
  REGION 1..5: note = CDR-H2 - Residues before
LEWIG

SEQ ID NO: 24 - skipped

SEQ ID NO: 25 - protein, 527 aa, Homo sapiens
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 26 - protein, 275 aa, Homo sapiens
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFR

SEQ ID NO: 27 - protein, 252 aa, Homo sapiens
IKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 28 - protein, 562 aa, Homo sapiens
  REGION 1..562: note = t-PA
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 29 - protein, 526 aa, synthetic construct
  REGION 1..526: note = Alteplase
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 30 - protein, 355 aa, synthetic construct
  REGION 1..355: note = Reteplase
SYQGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 31 - protein, 527 aa, synthetic construct
  REGION 1..527: note = Tenecteplase
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGNWSTAESGAECTNWQSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAAAAASPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

(19) United States
(12) Patent Application Publication
(10) Pub. No.: US 2025/0154252 A1
(43) Pub. Date: May 15, 2025

(54) TREATMENT OF CARDIOVASCULAR DISEASES USING ANTI-HUMAN GPVI ANTIBODIES

(71) Applicant: ACTICOR BIOTECH, Paris (FR)

(72) Inventors: Gilles AVENARD, Bordeaux (FR); Yannick PLETAN, Orsay (FR); Jean-Marie GROUIN, Feucherolles (FR); Sophie BINAY, Paris (FR); Elie TOLEDANO, Paris (FR); Andrea COMENDUCCI, Paris (FR)

(73) Assignee: ACTICOR BIOTECH, Paris (FR)

(21) Appl. No.: 18/840,227

(22) PCT Filed: Feb. 21, 2023
(86) PCT No.: PCT/EP2023/054333

§ 371 Date: Aug. 21, 2024

(30) Foreign Application Priority Data
Feb. 21, 2022 (EP) 22305190.5
May 6, 2022 (EP) 22305677.1

(51) Int. Cl. C07K 16/28; A61K 39/00; A61P 7/02

(52) U.S. Cl. CPC C07K 16/2803; A61P 7/02; A61K 2039/505; C07K 2317/24; C07K 2317/565

(57) ABSTRACT

An inhibitor of GPVI signaling pathway, in particular an isolated humanized protein binding to human Glycoprotein VI (hGPVI), for treating a GPVI-related condition in a subject in need thereof, in particular an acute ischemic stroke, including the administration of the inhibitor GPVI signaling pathway, in particular the anti-human glycoprotein VI antibody or fragment thereof, to a human patient in need thereof.

### FIELD OF INVENTION

The present invention relates to the treatment of cardiovascular diseases. In particular, the present invention relates to a method for treating a cardiovascular disease, in particular an acute ischemic stroke, comprising the administration of an inhibitor of the GPVI signaling pathway, in particular an anti-human glycoprotein VI antibody or fragment thereof, to a human patient in need thereof.

### BACKGROUND OF INVENTION

Acute coronary and cerebrovascular accidents are currently a major cause of death in the world. In addition, the global incidence of recurrence and death in the 6-month post-treatment period after an acute coronary syndrome is still 8-15%.

In the case of acute coronary syndrome with ST segment elevation, mechanical treatment with coronary angioplasty and introduction of a stent is highly efficient to urgently restore coronary artery flow, but does not prevent morbidity/mortality for about 15% of patients in the next 6 months. Thrombolytic treatments, which are based on long-term fibrinolytic, anticoagulant and anti-aggregating drugs associations, give even less encouraging results. Indeed, despite improvements in medical treatment of thrombosis, morbidity/mortality at 6 months is similar to that observed for acute coronary syndrome without ST segment elevation.

Regarding cerebrovascular ischemic accidents, treatments are still very limited due to the generally late caring of most patients and to the hemorrhagic risk of currently available anti-thrombotic treatments.

There is thus still a clinical need for improving treatments for cardiovascular diseases, and especially for new molecules with improved features compared to available molecules and for specific dosage regimens for administering said molecules. The challenge to face is to obtain molecules and protocols of administration with excellent efficiency on the pathological thrombosis but devoid of risk of bleeding.

In particular, there is still a need in the art for an improved treatment of GPVI-related conditions, in particular an acute ischemic stroke, without (or with low) occurrence of intracranial hemorrhages, aiming at reducing the risk of death of patients, in particular for old patients or patients with moderate to severe forms of the GPVI related conditions.

Glycoprotein VI has been demonstrated in animals to play a role in experimental thrombosis including stroke, vascular remodeling and to be critical in atherothrombosis. Contrary to αIIbβ3 integrin antagonists, which are currently used in thrombosis treatment and inhibit platelet final activation phase, i.e., platelet aggregation, and to antagonists of platelet recruitment (e.g., inhibitors of the P2Y12 ADP receptor and aspirin), GPVI is implicated into several steps of the platelet plug formation: initiation via the interaction with the injured vascular wall, amplification via initial platelet activation leading to the secretion of secondary agonists, the activation of integrins and of platelet procoagulant activity, growth and stabilization via the interaction with fibrin (Mammadova-Bach et al., 2015. Blood. 126 (5): 683-91). Thus, it was hypothesized that GPVI antagonists may prevent not only platelet aggregation, but also secondary agonists liberation as well as growth factors and cytokines secretion resulting into vascular lesions development. Finally, GPVI expression is limited to platelets and megakaryocytes, and thus represents a perfectly specific target for anti-thrombosis treatment.

A humanized antibody fragment with GPVI antagonist activity was described in WO2017/021539. This antibody fragment, named ACT017 or glenzocimab, was demonstrated to be an agent that possess anti-platelet activity and efficient antithrombotic potential in models of thrombosis, without increasing bleeding.

The Inventors herein demonstrate that this antibody fragment was also able to decrease intracranial hemorrhages and the risk of death of patients suffering from a stroke, which was never observed with an anti-platelet agent before. In addition, the Inventors have surprisingly found that said antibody fragment is particularly efficient in subpopulations of stroke patients. Another advantage is that this antibody fragment is compatible with thrombolytic agents, anticoagulants and/or antiplatelet agents.

### SUMMARY

The present invention relates to an isolated humanized protein binding to human Glycoprotein VI (hGPVI) for use in treating a GPVI-related condition in a subject in need thereof, wherein said subject presents at least one of the following conditions:
(i). said subject is affected with a moderate or severe GPVI-related condition,
(ii). said subject is of 65 years of age or older, and/or
(iii). said subject is undergoing or has undergone thrombectomy and thrombolysis, preferably wherein said GPVI-related condition is a stroke, more preferably an acute ischemic stroke.

In one embodiment, said subject presents at least one of the following conditions:
(i). said subject is affected with a moderate to severe GPVI-related condition, and/or
(ii). said subject is of 65 years of age or older, preferably wherein said GPVI-related condition is a stroke, more preferably an acute ischemic stroke.

In one embodiment, the isolated humanized protein binding to human Glycoprotein VI (hGPVI) for use as described hereinabove is an antibody molecule or an antibody fragment selected from the group consisting of a whole antibody, a single chain antibody, a Fv, a Fab, a unibody, a domain antibody, and a nanobody; or a monomeric antibody mimetic selected from the group consisting of an affibody, an affilin, an affitin, an adnectin, an atrimer, an evasin, a DARPin, an anticalin, an avimer, a fynomer, and a versabody, preferably the isolated humanized protein is a monovalent antibody or fragment thereof.

In one embodiment, the isolated humanized protein binding to hGPVI for use as described hereinabove is an antibody molecule or an antibody fragment, wherein
the variable region of the heavy chain comprises at least one of the following CDRs:

VH-CDR1:
(SEQ ID NO: 1)
GYTFTSYNMH;
VH-CDR2:
(SEQ ID NO: 2)
GIYPGNGDTSYNQKFQG;
and
VH-CDR3:
(SEQ ID NO: 3)
GTVVGDWYFDV;

or any CDR having an amino acid sequence that shares at least 60% of identity with SEQ ID NO: 1-3; and
the variable region of the light chain comprises at least one of the following CDRs:

VL-CDR1:
(SEQ ID NO: 4)
RSSQSLENSNGNTYLN;
VL-CDR2:
(SEQ ID NO: 5)
RVSNRFS;
and
VL-CDR3:
(SEQ ID NO: 6)
LQLTHVPWT;

or any CDR having an amino acid sequence that shares at least 60% of identity with SEQ ID NO: 4-6.

In one embodiment, the isolated humanized protein binding to hGPVI for use as described hereinabove is an antibody molecule or an antibody fragment, wherein the variable region of the heavy chain comprises the following CDRs: GYTFTSYNMH (SEQ ID NO: 1), GIYPGNGDTSYNQKFQG (SEQ ID NO: 2) and GTVVGDWYFDV (SEQ ID NO: 3) and the variable region of the light chain comprises the following CDRs: RSSQSLENSNGNTYLN (SEQ ID NO: 4), RVSNRFS (SEQ ID NO: 5) and LQLTHVPWT (SEQ ID NO: 6) or any CDR having an amino acid sequence that shares at least 60% of identity with said SEQ ID NO: 1-6.

In one embodiment, the isolated humanized protein binding to hGPVI for use as described hereinabove is an antibody molecule or an antibody fragment, wherein the amino acid sequence encoding the heavy chain variable region is SEQ ID NO: 7 and the amino acid sequence encoding the light variable region is SEQ ID NO: 8, or any sequence having an amino acid sequence that shares at least 60% of identity with said SEQ ID NO: 7 or 8.

In one embodiment, the isolated humanized protein binding to hGPVI for use as described hereinabove is an antibody molecule or an antibody fragment, wherein the amino acid sequence encoding the heavy chain variable region is SEQ ID NO: 7 and the amino acid sequence encoding the light variable region is SEQ ID NO: 9, or any sequence having an amino acid sequence that shares at least 60% of identity with said SEQ ID NO: 7 or 9.

In one embodiment, the GPVI-related condition is a stroke, and the severity of the stroke is evaluated by the NIH stroke scale (NIHSS), preferably said subject affected with a stroke has a NIHSS score at baseline equal to or greater than 10.

In one embodiment, said GPVI-related condition is an ischemic stroke, preferably an acute ischemic stroke.

In one embodiment, said subject is diagnosed with a stroke.

In one embodiment, said subject has received, is receiving or will receive a thrombolytic agent. In one embodiment, said subject has received, is receiving or will receive a thrombolytic agent, and/or an anticoagulant, and/or an antiplatelet agent.

In one embodiment, said subject is of 80 years of age or older.

In one embodiment, said protein prevents the occurrence of intracranial hemorrhages in the subject.

In one embodiment, said protein decreases the risk of death of the subject, preferably the risk of death within a time period of 20 days after the onset of the GPVI-related condition, more preferably the risk of death within a time period of 10 days after the onset of the GPVI-related condition, and even more preferably the risk of death within a time period of 5 days after the onset of the GPVI-related condition.

In one embodiment, said protein decreases the degree of disability or dependence following the GPVI-related condition.

The present invention further relates to an isolated humanized protein binding to human Glycoprotein VI (hGPVI) for use in decreasing the degree of disability or dependence following a GPVI-related condition, such as a stroke, in a subject, preferably for use in decreasing the risk of having a modified Rankin Scale (mRS) score measured 90 days after the administration of the isolated humanized protein equal to or greater than 4 or for use in increasing the probability of having a mRS score measured 90 days after the administration of the isolated humanized protein equal to or lower than 3.

The present invention further relates to an isolated humanized protein binding to human Glycoprotein VI (hGPVI) for use in preventing the occurrence of intracranial hemorrhages in a subject affected with a GPVI-related condition, wherein preferably said GPVI-related condition is a stroke, more preferably an acute ischemic stroke.

The present invention further relates to an isolated humanized protein binding to human Glycoprotein VI (hGPVI) for use in decreasing the risk of death in a subject affected with a GPVI-related condition, preferably for use in decreasing the risk of death within a time period of 20 days after the onset of the GPVI-related condition, more preferably within a time period of 10 days after the onset of the GPVI-related condition, and even more preferably the risk of death within a time period of 5 days after the onset of the GPVI-related condition, wherein preferably said GPVI-related condition is a stroke, more preferably an acute ischemic stroke.

### Definition

In the present invention, the following terms have the following meanings:

“Glycoprotein VI (GPVI)” is a platelet membrane glycoprotein that is involved in platelet-collagen interactions. GPVI is a transmembrane collagen receptor expressed on the surface of platelets. In one embodiment, the amino acid sequence of human GPVI is SEQ ID NO: 13 (accession number: BAA89353.1) or any amino acid sequence presenting at least about 90% identity with SEQ ID NO: 13, preferably at least about 91, 92, 93, 94, 95, 96, 97, 98, 99% identity or more with SEQ ID NO: 13.

(SEQ ID NO: 13)
MSPSPTALFCLGLCLGRVPA (Signal peptide).
QSGPLPKPSLQALPSSLVPLEKPVTLRCQGPPGVDLYRLEKLSSSRYQD
QAVLFIPAMKRSLAGRYRCSYQNGSLWSLPSDQLELVATGVFAKPSLSA
QPGPAVSSGGDVTLQCQTRYGFDQFALYKEGDPAPYKNPERWYRASFPI
ITVTAAHSGTYRCYSFSSRDPYLWSAPSDPLELVVTGTSVTPSRLPTEP
PSSVAEFSEATAELTVSFTNKVFTTETSRSITTSPKESDSPAGPARQYY
TKGN (Extracellular domain).
LVRICLGAVILIILAGFLAEDWHSRRKRLRHRGRAVQRPLPPLPPLPQT
RKSHGGQDGGRQDVHSRGLCS
(Transmembrane and cytoplasmic domains).

The extracellular domain of GPVI is composed of two Ig-like C2-type domains, namely D1 and D2, linked by a hinge interdomain. In one embodiment, D1 comprises amino acid residues 21 to 109 of SEQ ID NO: 13. In one embodiment, the hinge interdomain between D1 and D2 comprises amino acid residues 110 to 113 of SEQ ID NO: 13. In one embodiment, D2 comprises amino acid residues 114 to 207 of SEQ ID NO: 13.

“About” preceding a figure means plus or less 10% of the value of said figure.

“Antibody” or “Immunoglobulin”—As used herein, the term “immunoglobulin” includes a protein having a combination of two heavy and two light chains whether or not it possesses any relevant specific immunoreactivity. “Antibodies” refers to such assemblies which have significant known specific immunoreactive activity to an antigen of interest (e.g. human GPVI). The term “anti-GPVI antibodies” is used herein to refer to antibodies which exhibit immunological specificity for human GPVI protein. As explained elsewhere herein, “specificity” for human GPVI does not exclude cross-reaction with species homologues of GPVI. Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. All five classes of antibodies are within the scope of the present invention; the following discussion will generally be directed to the IgG class of immunoglobulin molecules. With regard to IgG, immunoglobulins comprise two identical light polypeptide chains of molecular weight of about 23,000 Daltons, and two identical heavy chains of molecular weight of about 53,000-70,000 Daltons. The four chains are joined by disulfide bonds in a “Y” configuration wherein the light chains bracket the heavy chains starting at the mouth of the “Y” and continuing through the variable region. The light chains of an antibody are classified as either kappa or lambda ([κ], [λ]). Each heavy chain class may be bonded with either a kappa or lambda light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the VH and VL chains.

“An isolated antibody”—As used herein, an “isolated antibody” is one that has been separated and/or recovered from a component of its natural environment. Contaminant components of its natural environment are materials that would interfere with diagnostic or therapeutic uses of the antibody, and may include enzymes, hormones, and other proteinaceous or non-proteinaceous components. In preferred embodiments, the antibody is purified: (1) to greater than 80, 85, 90, 91, 92, 93, 94, 95% or more by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity as shown by SDS-PAGE under reducing or non-reducing conditions and using Coomassie blue or, preferably, silver staining. Isolated antibody includes the antibody in situ within recombinant cells since at least one component of the antibody's natural environment will not be present. Ordinarily, however, isolated antibody will be prepared by at least one purification step.

“Affinity variants”—As used herein, the term “affinity variant” refers to a variant antibody which exhibits one or more changes in amino acid sequence compared to a reference anti-GPVI antibody, wherein the affinity variant exhibits an altered affinity for the human GPVI protein in comparison to the reference antibody. Typically, affinity variants will exhibit an improved affinity for human GPVI, as compared to the reference anti-GPVI antibody. The improvement may be either a lower K_{D} for human GPVI, a higher KA for human GPVI, a faster on-rate for human GPVI or a slower off-rate for human GPVI or an alteration in the pattern of cross-reactivity with non-human GPVI homologues. Affinity variants typically exhibit one or more changes in amino acid sequence (such as, for example in the CDRs), as compared to the reference anti-GPVI antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.

“Binding Site”—As used herein, the term “binding site” comprises a region of a protein which is responsible for selectively binding to a target antigen of interest (e.g. human GPVI). Binding domains or binding regions comprise at least one binding site. Exemplary binding domains include an antibody variable domain. The protein of the invention may comprise a single antigen binding site or multiple (e.g., two, three or four) antigen binding sites. Preferably, however, the protein of the invention comprises a single antigen binding site.

“Conservative amino acid substitution”—As used herein, “conservative amino acid substitutions” are ones in which the amino acid residue is replaced with an amino acid residue that has similar properties, such that one skilled in the art of peptide chemistry would expect the secondary structure and hydropathic nature of the polypeptide to be substantially unchanged. Amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Other families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members.

“Chimeric”—As used herein, a “chimeric” protein comprises a first amino acid sequence linked to a second amino acid sequence with which it is not naturally linked in nature. The amino acid sequences may normally exist in separate proteins that are brought together in the fusion protein or they may normally exist in the same protein but are placed in a new arrangement in the fusion protein. A chimeric protein may be created, for example, by chemical synthesis, or by creating and translating a polynucleotide in which the peptide regions are encoded in the desired relationship.

“CDR”—As used herein, the term “CDR” or “complementarity determining region” means the non-contiguous antigen combining sites found within the variable region of both heavy and light chain polypeptides. CDRs were identified according to the following rules as deduced from Kabat (Kabat et al., 1991. J. Immunol. 147 (5): 1709-19) and Chothia & Lesk (Chothia C. and A. M. Lesk, 1987. J. Mol. Biol. 196 (4): 901-17):

### CDR-L1:

Start-Approx residue 24;
Residue before is always a Cys;
Residue after is always a Trp. Typically TRP-TYR-GLN, but also, TRP-LEU-GLN, TRP-PHE-GLN, TRP-TYR-LEU;
Length 10 to 17 residues;

### CDR-L2:

Start-always 16 residues after the end of L1;
Residues before generally ILE-TYR, but also, VAL-TYR, ILE-LYS, ILE-PHE;
Length always 7 residues;

### CDR-L3:

Start—always 33 residues after end of L2;
Residue before is always Cys;
Residues after always PHE-GLY-XXX-GLY (SEQ ID NO: 21); Length 7 to 11 residues;

### CDR-H1:

Start-Approx residue 26 (always 4 after a CYS) [Chothia/AbM definition] Kabat definition starts 5 residues later;
Residues before always CYS-XXX-XXX-XXX (SEQ ID NO: 22);
Residues after always a TRP. Typically TRP-VAL, but also, TRP-ILE, TRP-ALA
Length 10 to 12 residues (AbM definition) Chothia definition excludes the last 4 residues;

### CDR-H2:

Start-always 15 residues after the end of Kabat/AbM definition) of CDR-H1 Residues before typically LEU-GLU-TRP-ILE-GLY (SEQ ID NO: 23), but a number of variations;
Residues after LYS/ARG-LEU/ILE/VAL/PHE/THR/ALA-THR/SER/ILE/ALA Length Kabat definition 16 to 19 residues (AbM definition ends 7 residues earlier);

### CDR-H3:

Start-always 33 residues after end of CDR-H2 (always 2 after a CYS);
Residues before always CYS-XXX-XXX (typically CYS-ALA-ARG);
Residues after always TRP-GLY-XXX-GLY (SEQ ID NO: 24);
Length 3 to 25 residues.

“CH2 domain”—As used herein, the term “CH2 domain” includes the region of a heavy chain molecule that usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton, Molec. Immunol. 22 (1985)161-206).

“Derived from”—As used herein, the term “derived from” a designated protein (e.g., an anti-GPVI antibody or antigen-binding fragment thereof) refers to the origin of the protein. In an embodiment, the protein or amino acid sequence which is derived from a particular starting protein is a CDR sequence or sequence related thereto. In an embodiment, the amino acid sequence which is derived from a particular starting protein is not contiguous. For example, in an embodiment, one, two, three, four, five, or six CDRs are derived from a starting antibody. In an embodiment, the protein or amino acid sequence which is derived from a particular starting protein or amino acid sequence has an amino acid sequence that is essentially identical to that of the starting sequence, or a region thereof wherein the region consists of at least 3-5 amino acids, at least 5-10 amino acids, at least 10-20 amino acids, at least 20-30 amino acids, or at least 30-50 amino acids, or which is otherwise identifiable to one of ordinary skill in the art as having its origin in the starting sequence. In an embodiment, the one or more CDR sequences derived from the starting antibody are altered to produce variant CDR sequences, e.g., affinity variants, wherein the variant CDR sequences maintain GPVI binding activity.

“Diabodies”—As used herein, the term “diabodies” refers to small antibody fragments prepared by constructing sFv fragments (see sFv paragraph) with short linkers (about 5-residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 0404097; WO 1993/011161; and Holliger (Holliger et al., 1993. Proc. Natl. Acad. Sci. 90 (14): 6444-6448).

“Engineered”—As used herein, the term “engineered” includes manipulation of nucleic acid or polypeptide molecules by synthetic means (e.g., by recombinant techniques, in vitro peptide synthesis, by enzymatic or chemical coupling of peptides or some combination of these techniques). Preferably, the antibodies of the invention are engineered, including for example, humanized and/or chimeric antibodies, and antibodies which have been engineered to improve one or more properties, such as antigen binding, stability/half-life or effector function.

“Epitope”—As used herein, the term “epitope” refers to a specific arrangement of amino acids located on a protein or proteins to which an antibody binds. Epitopes often consist of a chemically active surface grouping of molecules such as amino acids or sugar side chains, and have specific three dimensional structural characteristics as well as specific charge characteristics. Epitopes can be linear or conformational, i.e., involving two or more sequences of amino acids in various regions of the antigen that may not necessarily be contiguous.

“Framework region”—As used herein, the term “framework region” or “FR region” includes the amino acid residues that are part of the variable region, but are not part of the CDRs (e.g., using the Kabat/Chothia definition of CDRs). Therefore, a variable region framework is between about 100-120 amino acids in length but includes only those amino acids outside of the CDRs. For the specific example of a heavy chain variable region and for the CDRs as defined by Kabat/Chothia, framework region 1 may correspond to the domain of the variable region encompassing amino acids 1-25; framework region 2 may correspond to the domain of the variable region encompassing amino acids 36-49; framework region 3 may correspond to the domain of the variable region encompassing amino acids 67-98, and framework region 4 may correspond to the domain of the variable region from amino acids 110 to the end of the variable region. The framework regions for the light chain are similarly separated by each of the light chain variable region CDRs. In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainders of the heavy and light variable domains show less inter-molecular variability in amino acid sequence and are termed the framework regions. The framework regions largely adopt a [beta]-sheet conformation and the CDRs form loops which connect, and in some cases form part of, the [beta]-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Fragment”—As used herein, the term “fragment” refers to a part or region of an antibody or antibody chain comprising fewer amino acid residues than an intact or complete antibody or antibody chain. The term “antigen-binding fragment” refers to a protein fragment of an immunoglobulin or antibody that binds antigen or competes with intact antibody (i.e., with the intact antibody from which they were derived) for antigen binding (i.e., specific binding to human GPVI). As used herein, the term “fragment” of an antibody molecule includes antigen-binding fragments of antibodies, for example, an antibody light chain variable domain (VL), an antibody heavy chain variable domain (VH), a single chain antibody (scFv), a F(ab′)₂ fragment, a Fab fragment, an Fd fragment, an Fv fragment, a single domain antibody fragment (DAb), a one-armed (monovalent) antibody, diabodies or any antigen-binding molecule formed by combination, assembly or conjugation of such antigen binding fragments. Fragments can be obtained, e.g., via chemical or enzymatic treatment of an intact or complete antibody or antibody chain or by recombinant means.

The “Fc” fragment of an antibody comprises the carboxy-terminal portions of both H chains held together by disulfides. The effector functions of antibodies are determined by sequences in the Fc region, which region is also the part recognized by Fc receptors (FcR) found on certain types of cells.

“Fv”—As used herein, the term “Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Heavy chain region”—As used herein, the term “heavy chain region” includes amino acid sequences derived from the constant domains of an immunoglobulin heavy chain. A protein comprising a heavy chain region comprises at least one of: a CH1 domain, a hinge (e.g., upper, middle, and/or lower hinge region) domain, a CH2 domain, a CH3 domain, or a variant or fragment thereof. In an embodiment, a binding molecule of the invention may comprise the Fc region of an immunoglobulin heavy chain (e.g., a hinge portion, a CH2 domain, and a CH3 domain). In another embodiment, a binding molecule of the invention lacks at least a region of a constant domain (e.g., all or part of a CH2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, CH2 and CH3 domain sequences from a human immunoglobulin). In certain embodiments, the constituent constant domains of the heavy chain region are from different immunoglobulin molecules. For example, a heavy chain region of a protein may comprise a CH2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the proteins of the invention disclosed herein may comprise alterations or modifications to one or more of the heavy chain constant domains (CH1, hinge, CH2 or CH3) and/or to the light chain constant domain (CL). Exemplary modifications include additions, deletions or substitutions of one or more amino acids in one or more domains.

“Hinge region”—As used herein, the term “hinge region” includes the region of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J. Immunol. 161 (8): 4083-90).

The terms “hypervariable loop” and “complementarity determining region” are not strictly synonymous, since the hypervariable loops (HVs) are defined on the basis of structure, whereas complementarity determining regions (CDRs) are defined based on sequence variability (Kabat, Elvin A. (1983). Sequences of proteins of immunological interest (5^{th} edition). Besthesda, MD: Public Health Service, National Institutes of Health) and the limits of the HVs and the CDRs may be different in some VH and VL domains. The CDRs of the VL and VH domains can typically be defined by the Kabat/Chothia definition (see above). In one embodiment, the CDRs of the VL and VH domains may comprise the following amino acids: residues 24-39 (CDRL1), 55-61 (CDRL2) and 94-102 (CDRL3) in the light chain variable domain, and residues 26-35 (CDRH1), 50-66 (CDRH2) and 99-109 (CDRH3) in the heavy chain variable domain. Thus, the HVs may be comprised within the corresponding CDRs and references herein to the “hypervariable loops” of VH and VL domains should be interpreted as also encompassing the corresponding CDRs, and vice versa, unless otherwise indicated. The more highly conserved regions of variable domains are called the framework region (FR), as defined herein. The variable domains of native heavy and light chains each comprise four FRs (FR1, FR2, FR3 and FR4, respectively), largely adopting a [beta]-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., 1992. J. Mol. Biol. 227 (3): 799-817; Tramontano et al., 1990. J. Mol. Biol. 215 (1): 175-182). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

“Humanized”—As used herein, the term “humanized” refers to chimeric immunoglobulins, immunoglobulin chains or fragments thereof (such as Fv, Fab, Fab′, or other antigen-binding subsequences of antibodies) which contain minimal sequence derived from a murine immunoglobulin. For example, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

“Humanizing substitutions”—As used herein, the term “humanizing substitutions” refers to amino acid substitutions in which the amino acid residue present at a particular position in the VH or VL domain of a non-human anti-GPVI antibody (for example a murine anti-GPVI antibody) is replaced with an amino acid residue which occurs at an equivalent position in a reference human VH or VL domain. The reference human VH or VL domain may be a VH or VL domain encoded by the human germline, in which case the substituted residues may be referred to as “germlining substitutions”. Humanizing/germlining substitutions may be made in the framework regions and/or the CDRs of an anti-GPVI antibody, defined herein.

“High human homology”—An antibody comprising a heavy chain variable domain (VH) and a light chain variable domain (VL) will be considered as having high human homology if the VH domains and the VL domains, taken together, exhibit at least 70, 75, 80, 85, 90, 95% or more amino acid sequence identity to the closest matching human germline VH and VL sequences. Antibodies having high human homology may include antibodies comprising VH and VL domains of native non-human antibodies which exhibit sufficiently high % sequence identity human germline sequences, as well as engineered, especially humanized, variants of such antibodies and also “fully human” antibodies. In an embodiment the VH domain of the antibody with high human homology may exhibit an amino acid sequence identity or sequence homology of 75%, 80% or greater with one or more human VH domains across the framework regions FR1, FR2, FR3 and FR4. In other embodiments the amino acid sequence identity or sequence homology between the VH domain of the protein of the invention and the closest matching human germline VH domain sequence may be 85% or greater, 90% or greater, 95% or greater, 97% or greater, or up to 99% or even 100%. In an embodiment the VH domain of the antibody with high human homology may contain one or more (e.g., 1 to 20) amino acid sequence mismatches across the framework regions FR1, FR2, FR3 and FR4, in comparison to the closest matched human VH sequence. In another embodiment the VL domain of the antibody with high human homology may exhibit a sequence identity or sequence homology of 80% or greater with one or more human VL domains across the framework regions FR1, FR2, FR3 and FR4. In other embodiments the amino acid sequence identity or sequence homology between the VL domain of the protein of the invention and the closest matching human germline VL domain sequence may be 85% or greater 90% or greater, 95% or greater, 97% or greater, or up to 99% or even 100%.

In an embodiment the VL domain of the antibody with high human homology may contain one or more (e.g., 1 to 20, preferably 1 to 10 and more preferably 1 to 5) amino acid sequence mismatches across the framework regions FR1, FR2, FR3 and FR4, in comparison to the closest matched human VL sequence. Before analyzing the percentage sequence identity between the antibody with high human homology and human germline VH and VL, the canonical folds may be determined, which allow the identification of the family of human germline segments with the identical combination of canonical folds for H1 and H2 or L1 and L2(and L3). Subsequently the human germline family member that has the highest degree of sequence homology with the variable region of the antibody of interest is chosen for scoring the sequence homology. The determination of Chothia canonical classes of hypervariable loops L1, L2, L3, H1 and H2 can be performed with the bioinformatics tools publicly available on webpage www.bioinf.org.uk/abs/chothia.html.page. The output of the program shows the key residue requirements in a data file. In these data files, the key residue positions are shown with the allowed amino acids at each position. The sequence of the variable region of the antibody of interest is given as input and is first aligned with a consensus antibody sequence to assign the Kabat/Chothia numbering scheme. The analysis of the canonical folds uses a set of key residue templates derived by an automated method developed by Martin and Thornton (Martin et al., 1996. J. Mol. Biol. 263 (5): 800-815). With the particular human germline V segment known, which uses the same combination of canonical folds for H1 and H2 or L1 and L2 (and L3), the best matching family member in terms of sequence homology can be determined. With bioinformatics tools the percentage sequence identity between the VH and VL domain framework amino acid sequences of the antibody of interest and corresponding sequences encoded by the human germline can be determined, but actually manual alignment of the sequences can be applied as well. Human immunoglobulin sequences can be identified from several protein data bases, such as VBase (http://vbase.mrc-cpe.cam.ac.uk/) or the Pluckthun/Honegger database (http://www.bioc.unizh.ch/antibody/Sequences/Germlines). To compare the human sequences to the V regions of VH or VL domains in an antibody of interest a sequence alignment algorithm such as available via websites like www.expasy.ch/tools/#align can be used, but also manual alignment with the limited set of sequences can be performed. Human germline light and heavy chain sequences of the families with the same combinations of canonical folds and with the highest degree of homology with the framework regions 1, 2, and 3 of each chain are selected and compared with the variable region of interest; also the FR4 is checked against the human germline JH and JK or JL regions. Note that in the calculation of overall percent sequence homology the residues of FR1, FR2 and FR3 are evaluated using the closest match sequence from the human germline family with the identical combination of canonical folds. Only residues different from the closest match or other members of the same family with the same combination of canonical folds are scored (NB-excluding any primer-encoded differences). However, for the purposes of humanization, residues in framework regions identical to members of other human germline families, which do not have the same combination of canonical folds, can be considered “human”, despite the fact that these are scored “negative” according to the stringent conditions described above. This assumption is based on the “mix and match” approach for humanization, in which each of FR1, FR2, FR3 and FR4 is separately compared to its closest matching human germline sequence and the humanized molecule therefore contains a combination of different FRs as was done by Qu and colleagues (Qu et al., Clin. Cancer Res. 5:3095-3100 (1999)) and Ono and colleagues (Ono et al., Mol. Immunol. 36:387-395 (1999)). The boundaries of the individual framework regions may be assigned using the IMGT numbering scheme, which is an adaptation of the numbering scheme of Chothia (Lefranc et al., Nucleic acid res 27:209-212 (1999); http://im.gt.cines.fr). Antibodies with high human homology may comprise hypervariable loops or CDRs having human or human-like canonical folds, as discussed in detail below. In an embodiment at least one hypervariable loop or CDR in either the VH domain or the VL domain of the antibody with high human homology may be obtained or derived from a VH or VL domain of a non-human antibody, yet exhibit a predicted or actual canonical fold structure which is substantially identical to a canonical fold structure which occurs in human antibodies. It is well established in the art that although the primary amino acid sequences of hypervariable loops present in both VH domains and VL domains encoded by the human germline are, by definition, highly variable, all hypervariable loops, except CDR H3 of the VH domain, adopt only a few distinct structural conformations, termed canonical folds (Chothia et al., J. Mol. Biol. 196:901-917 (1987); Tramontano et al., Proteins 6:382-94 (1989)), which depend on both the length of the hypervariable loop and presence of the so-called canonical amino acid residues (Chothia et al., J. Mol. Biol. 196:901-917 (1987)). Actual canonical structures of the hypervariable loops in intact VH or VL domains can be determined by structural analysis (e.g., X-ray crystallography), but it is also possible to predict canonical structure on the basis of key amino acid residues which are characteristic of a particular structure (discussed further below). In essence, the specific pattern of residues that determines each canonical structure forms a “signature” which enables the canonical structure to be recognized in hypervariable loops of a VH or VL domain of unknown structure; canonical structures can therefore be predicted on the basis of primary amino acid sequence alone. The predicted canonical fold structures for the hypervariable loops of any given VH or VL sequence in an antibody with high human homology can be analyzed using algorithms which are publicly available from www.bioinf.org.uk/abs/chothia.html, www.biochem.ucl.ac.uk/˜martin/antibodies.html and www.bioc.unizh.ch/antibody/Sequences/Germlines/Vbase_hVk.html. These tools permit query VH or VL sequences to be aligned against human VH or VL domain sequences of known canonical structure, and a prediction of canonical structure made for the hypervariable loops of the query sequence. In the case of the VH domain, H1 and H2 loops may be scored as having a canonical fold structure “substantially identical” to a canonical fold structure known to occur in human antibodies if at least the first, and preferable both, of the following criteria are fulfilled:

1. An identical length, determined by the number of residues, to the closest matching human canonical structural class.

2. At least 33% identity, preferably at least 50% identity with the key amino acid residues described for the corresponding human H1 and H2 canonical structural classes (note for the purposes of the foregoing analysis the H1 and H2 loops are treated separately and each compared against its closest matching human canonical structural class). The foregoing analysis relies on prediction of the canonical structure of the H1 and H2 loops of the antibody of interest. If the actual structures of the H1 and H2 loops in the antibody of interest are known, for example based on X-ray crystallography, then the H1 and H2 loops in the antibody of interest may also be scored as having a canonical fold structure “substantially identical” to a canonical fold structure known to occur in human antibodies if the length of the loop differs from that of the closest matching human canonical structural class (typically by +1 or +2 amino acids) but the actual structure of the H1 and H2 loops in the antibody of interest matches the structure of a human canonical fold. Key amino acid residues found in the human canonical structural classes for the first and second hypervariable loops of human VH domains (H1 and H2) are described by Chothia et al., J. Mol. Biol. 227:799-817 (1992), the contents of which are incorporated herein in their entirety by reference. In particular, Table 3 on page 802 of Chothia et al., which is specifically incorporated herein by reference, lists preferred amino acid residues at key sites for H1 canonical structures found in the human germline, whereas Table 4 on page 803, also specifically incorporated by reference, lists preferred amino acid residues at key sites for CDR H2 canonical structures found in the human germline. In an embodiment, both HI and H2 in the VH domain of the antibody with high human homology exhibit a predicted or actual canonical fold structure which is substantially identical to a canonical fold structure which occurs in human antibodies. Antibodies with high human homology may comprise a VH domain in which the hypervariable loops H1 and H2 form a combination of canonical fold structures which is identical to a combination of canonical structures known to occur in at least one human germline VH domain. It has been observed that only certain combinations of canonical fold structures at H1 and H2 actually occur in VH domains encoded by the human germline. In an embodiment H1 and H2 in the VH domain of the antibody with high human homology may be obtained from a VH domain of a non-human species, yet form a combination of predicted or actual canonical fold structures which is identical to a combination of canonical fold structures known to occur in a human germline or somatically mutated VH domain. In non-limiting embodiments H1 and H2 in the VH domain of the antibody with high human homology may be obtained from a VH domain of a non-human species, and form one of the following canonical fold combinations: 1-1, 1-2, 1-3, 1-6, 1-4, 2-1, 3-1 and 3-5. An antibody with high human homology may contain a VH domain which exhibits both high sequence identity/sequence homology with human VH, and which contains hypervariable loops exhibiting structural homology with human VH. It may be advantageous for the canonical folds present at H1 and H2 in the VH domain of the antibody with high human homology, and the combination thereof, to be “correct” for the human VH germline sequence which represents the closest match with the VH domain of the antibody with high human homology in terms of overall primary amino acid sequence identity. By way of example, if the closest sequence match is with a human germline VH3 domain, then it may be advantageous for H1 and H2 to form a combination of canonical folds which also occurs naturally in a human VH3 domain. This may be particularly important in the case of antibodies with high human homology which are derived from non-human species, e.g., antibodies containing VH and VL domains which are derived from camelid conventional antibodies, especially antibodies containing humanized camelid VH and VL domains. Thus, in an embodiment the VH domain of the anti-GPVI antibody with high human homology may exhibit a sequence identity or sequence homology of 70% or greater, 80% or greater, 85% or greater, 90% or greater, 95% or greater, 97% or greater, or up to 99% or even 100% with a human VH domain across the framework regions FR1, FR2, FR3 and FR4, and in addition H1 and H2 in the same antibody are obtained from a non-human VH domain, but form a combination of predicted or actual canonical fold structures which is the same as a canonical fold combination known to occur naturally in the same human VH domain. In other embodiments, L1 and L2 in the VL domain of the antibody with high human homology are each obtained from a VL domain of a non-human species, and each exhibits a predicted or actual canonical fold structure which is substantially identical to a canonical fold structure which occurs in human antibodies. As with the VH domains, the hypervariable loops of VL domains of both VLambda and VKappa types can adopt a limited number of conformations or canonical structures, determined in part by length and also by the presence of key amino acid residues at certain canonical positions. Within an antibody of interest having high human homology, L1, L2 and L3 loops obtained from a VL domain of a non-human species, e.g., a Camelidae species, may be scored as having a canonical fold structure “substantially identical” to a canonical fold structure known to occur in human antibodies if at least the first, and preferable both, of the following criteria are fulfilled:

1. An identical length, determined by the number of residues, to the closest matching human structural class.

2. At least 33% identity, preferably at least 50% identity with the key amino acid residues described for the corresponding human L1 or L2 canonical structural classes, from either the VLambda or the VKappa repertoire (note for the purposes of the foregoing analysis the L1 and L2 loops are treated separately and each compared against its closest matching human canonical structural class). The foregoing analysis relies on prediction of the canonical structure of the L1, L2 and L3 loops in the VL domain of the antibody of interest. If the actual structure of the L1, L2 and L3 loops is known, for example based on X-ray crystallography, then L1, L2 or L3 loops derived from the antibody of interest may also be scored as having a canonical fold structure “substantially identical” to a canonical fold structure known to occur in human antibodies if the length of the loop differs from that of the closest matching human canonical structural class (typically by +1 or +2 amino acids) but the actual structure of the loops in the antibody of interest matches a human canonical fold. Key amino acid residues found in the human canonical structural classes for the CDRs of human VLambda and VKappa domains are described by Morea et al., Methods, 20:267-279 (2000) and Martin et al., J. Mol. Biol., 263:800-815 (1996). The structural repertoire of the human VKappa domain is also described by Tomlinson et al., EMBO J. 14:4628-4638 (1995), and that of the VLambda domain by Williams et al., J. Mol. Biol., 264:220-232 (1996). The contents of all these documents are to be incorporated herein by reference. L1 and L2 in the VL domain of an antibody with high human homology may form a combination of predicted or actual canonical fold structures which is identical to a combination of canonical fold structures known to occur in a human germline VL domain. In non-limiting embodiments LI and L2 in the VLambda domain of an antibody with high human homology may form one of the following canonical fold combinations: 11-7, 13-7 (A,B,C), 14-7 (A,B), 12-11, 14-11 and 12-12 (as defined in Williams et al., J. Mol. Biol. 264:220-32 (1996) and as shown on http://www.bioc.uzh.ch/antibody/Sequences/Germlines/VBase_hVL.html). In non-limiting embodiments L1 and L2 in the VKappa domain may form one of the following canonical fold combinations: 2-1, 3-1, 4-1 and 6-1 (as defined in Tomlinson et al., EMBO J. 14:4628-38 as shown on (1995) and http://www.bioc.uzh.ch/antibody/Sequences/Germlines/VBase_hVK.html).

In a further embodiment, all three of L1, L2 and L3 in the VL domain of an antibody with high human homology may exhibit a substantially human structure. It is preferred that the VL domain of the antibody with high human homology exhibit both high sequence identity/sequence homology with human VL, and also that the hypervariable loops in the VL domain exhibit structural homology with human VL.

In an embodiment, the VL domain of the anti-GPVI antibody with high human homology may exhibit a sequence identity of 70% or greater, 80% or greater, 85% or greater, 90% or greater, 95% or greater, 97% or greater, or up to 99% or even 100% with a human VL domain across the framework regions FR1, FR2, FR3 and FR4, and in addition hypervariable loop L1 and hypervariable loop L2 may form a combination of predicted or actual canonical fold structures which is the same as a canonical fold combination known to occur naturally in the same human VL domain. It is, of course, envisaged that VH domains exhibiting high sequence identity/sequence homology with human VH, and also structural homology with hypervariable loops of human VH will be combined with VL domains exhibiting high sequence identity/sequence homology with human VL, and also structural homology with hypervariable loops of human VL to provide antibodies with high human homology containing VH/VL pairings with maximal sequence and structural homology to human-encoded VH/VL pairings.

“Immunospecific”, “specific for” or to “specifically bind”—As used herein, an antibody is said to be “immunospecific”, “specific for” or to “specifically bind” an antigen if it reacts at a detectable level with the antigen, preferably with an affinity constant, KA, of greater than or equal to about 10⁶ M⁻¹, greater than or equal to about 10⁷ M⁻¹, or greater than or equal to 10⁸ M⁻¹, or greater than or equal to 1.5 10⁸ M⁻¹., or greater than or equal to 10⁹ M⁻¹ or greater than or equal to 5 10⁹ M⁻¹. Affinity of an antibody for its cognate antigen is also commonly expressed as a dissociation constant K_{D}, and in certain embodiments, an antibody specifically binds to antigen if it binds with a K_{D} of less than or equal to 10⁻⁶ M, less than or equal to 10⁻⁷ M, or less than or equal to 1.5 10⁻⁸ M, or less than or equal to 10⁻⁸ M, or less than or equal to 5 10⁻⁹ M or less than or equal to 10⁻⁹ M. Affinities of antibodies can be readily determined using conventional techniques, for example, those described by Scatchard G et al. (The attractions of proteins for small molecules and ions. Ann NY Acad Sci 1949; 51:660-672). Binding properties of an antibody to antigens, cells or tissues thereof may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, BIAcore).

“Inhibitor of GPVI signaling pathway”—As used herein, this term includes any agent (e.g., chemical entity, protein, and the like) that, upon administration to a patient or to a cell induces an inhibition or down-regulation of a biological activity associated with activation of the GPVI signaling pathway, including any of the downstream biological effects otherwise resulting from the binding of the GPVI to its natural ligand.

“Isolated nucleic acid”—As used herein, an “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

The term “polypeptide” is used in its conventional meaning, i.e., as a sequence of less than 100 amino acids. A polypeptide usually refers to a monomeric entity. The term “protein” refers to a sequence of more than 100 amino acids and/or to a multimeric entity. The proteins of the invention are not limited to a specific length of the product. This term does not refer to or exclude post-expression modifications of the protein, for example, glycosylation, acetylation, phosphorylation and the like, as well as other modifications known in the art, both naturally occurring and non-naturally occurring. A protein may be an entire protein, or a subsequence thereof. Particular proteins of interest in the context of this invention are amino acid subsequences comprising CDRs and being capable of binding an antigen. An “isolated protein” is one that has been identified and separated and/or recovered from a component of its natural environment. In preferred embodiments, the isolated protein will be purified (1) to greater than 80, 85, 90, 95% by weight of protein as determined by the Lowry method, and most preferably more than 96, 97, 98, or 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver staining. Isolated protein includes the protein in situ within recombinant cells since at least one component of the protein's natural environment will not be present. Ordinarily, however, isolated protein will be prepared by at least one purification step.

“Identity” or “identical”—As used herein, the term “identity” or “identical”, when used in a relationship between the sequences of two or more amino acid sequences, refers to the degree of sequence relatedness between amino acid sequences, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related amino acid sequences can be readily calculated by known methods. Such methods include, but are not limited to, those described in Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et al., 1988. SIAM J. Appl. Math. 48 (5): 1073-1082. Preferred methods for determining identity are designed to give the largest match between the sequences tested. Methods of determining identity are described in publicly available computer programs. Preferred computer program methods for determining identity between two sequences include the GCG program package, including GAP (Devereux et al., 1984. Nucl. Acid. Res. 12 (1 Pt 1): 387-395; Genetics Computer Group, University of Wisconsin Biotechnology Center, Madison, WI), BLASTP, BLASTN, TBLASTN and FASTA (Altschul et al., 1990. J. Mol. Biol. 215 (3): 403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990. J. Mol. Biol. 215 (3): 403-410). The well-known Smith Waterman algorithm may also be used to determine identity.

“Modified antibody”—As used herein, the term “modified antibody” includes synthetic forms of antibodies which are altered such that they are not naturally occurring, e.g., antibodies that comprise at least two heavy chain regions but not two complete heavy chains (such as, domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a modified antibody of the invention is a fusion protein comprising at least one heavy chain region lacking a CH2 domain and comprising a binding domain of a protein comprising the binding region of one member of a receptor ligand pair.

“Mammal”—As used herein, the term “mammal” refers to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

“Moderate to severe”—As used herein, the term “moderate to severe” when relating to a GPVI related condition, such as, for example, a stroke, refers to the severity of the GPVI-related condition. The severity of the stroke may be, for example, assessed by the NIHSS score. In one embodiment, this term encompasses moderate stroke according to the NIHSS score, moderate to severe stroke according to the NIHSS score and severe stroke according to the NIHSS score.

“Monoclonal antibody”—As used herein, the term “monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

“Native sequence”—As used herein, the term “native sequence” nucleotide refers to a polynucleotide that has the same nucleotide sequence as a polynucleotide derived from nature. Accordingly, a “native sequence” protein is one that has the same amino acid sequence as a protein (e.g., antibody) derived from nature (e.g., from any species). Such native sequence polynucleotides and proteins can be isolated from nature or can be produced by recombinant or synthetic means. A polynucleotide “variant”, as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences as described herein and evaluating one or more biological activities of the encoded proteins as described herein and/or using any of a number of techniques well known in the art. A protein “variant”, as the term is used herein, is a protein that typically differs from a protein specifically disclosed herein in one or more substitutions, deletions, additions and/or insertions. Such variants may be naturally occurring or may be synthetically generated, for example, by modifying one or more of the above protein sequences and evaluating one or more biological activities of the protein as described herein and/or using any of a number of techniques well known in the art. Modifications may be made in the structure of the polynucleotides and proteins of the present invention and still obtain a functional molecule that encodes a variant or derivative protein with desirable characteristics. When it is desired to alter the amino acid sequence of a protein to create an equivalent, or even an improved, variant or region of a protein as described herein, one skilled in the art will typically change one or more of the codons of the encoding DNA sequence. For example, certain amino acids may be substituted for other amino acids in a protein structure without appreciable loss of its ability to bind other proteins (e.g., antigens) or cells. Since it is the binding capacity and nature of a protein that defines that protein's biological functional activity, certain amino acid sequence substitutions can be made in a protein sequence, and of course, its underlying DNA coding sequence, and nevertheless obtain a protein with similar properties. It is thus contemplated that various changes may be made in the amino acid sequences of the disclosed compositions, or corresponding DNA sequences that encode said proteins without appreciable loss of their biological utility or activity. In many instances, a protein variant will contain one or more conservative substitutions. A “conservative substitution” is one in which an amino acid is substituted for another amino acid that has similar properties, such that one skilled in the art of peptide chemistry would expect the secondary structure and hydropathic nature of the protein to be substantially unchanged. As outlined above, amino acid substitutions are generally therefore based on the relative similarity of the amino acid side-chain substituents, for example, their hydrophobicity, hydrophilicity, charge, size, and the like. Exemplary substitutions that take several of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain non-conservative changes. In a preferred embodiment, variant proteins differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the protein.

“Pharmaceutically acceptable excipient”—As used herein, the term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

“Specificity”—As used herein, the term “specificity” refers to the ability to specifically bind (e.g., immunoreact with) a given target, e.g., GPVI. A protein may be monospecific and contain one or more binding sites which specifically bind a target, or a protein may be multispecific and contain two or more binding sites which specifically bind the same or different targets. In an embodiment, an antibody as described herein is specific for more than one target. For example, in an embodiment, a multispecific binding molecule binds to GPVI and a second molecule.

“Single-chain Fv” also abbreviated as “sFv” or “scFv”—As used herein, the terms “Single-chain Fv”, “sFv” or “scFv” are antibody fragments that comprise the VH and VL antibody domains connected into a single amino acid chain. Preferably, the sFv amino acid sequence further comprises a peptidic linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

“Subject”—As used herein, the term “subject” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of a disease.

“Synthetic”—As used herein, the term “synthetic” with respect to proteins includes proteins which comprise an amino acid sequence that is not naturally occurring. For example, non-naturally occurring proteins are modified forms of naturally occurring proteins (e.g., comprising a mutation such as an addition, substitution or deletion) or proteins which comprise a first amino acid sequence (which may or may not be naturally occurring) that is linked in a linear sequence of amino acids to a second amino acid sequence (which may or may not be naturally occurring) to which it is not naturally linked in nature.

“Therapeutically effective amount” means level or amount of agent that is aimed at, without causing significant negative or adverse side effects to the target, (1) delaying or preventing the onset of GPVI-related disease; (2) slowing down or stopping the progression, aggravation, or deterioration of one or more symptoms of the GPVI-related disease; (3) bringing about ameliorations of the symptoms of the GPVI-related disease; (4) reducing the severity or incidence of the GPVI-related disease; or (5) curing the GPVI-related disease. A therapeutically effective amount may be administered prior to the onset of the GPVI-related disease, for a prophylactic or preventive action. Alternatively or additionally, the therapeutically effective amount may be administered after initiation of the GPVI-related disease, for a therapeutic action.

“Tissue-type plasminogen activator” or “t-PA”—As used herein, the term refers to a serine protease. It is thus one of the essential components of the dissolution of blood clots. Its primary function includes catalyzing the conversion of plasminogen to plasmin, the primary enzyme involved in dissolving blood clots. Examples of these drugs include, without limitation, alteplase, reteplase, and tenecteplase. Said t-PA may be a native sequence, which can be isolated from nature or can be produced by recombinant or synthetic means, or a variant thereof that retain the enzymatic and/or fibrinolytic activity of native t-PA. An example of a human t-PA (in particular the premature form) is the sequence SEQ ID NO: 28.

(SEQ ID NO: 28)
MDAMKRGLCCVLLLCGAVFVSPSQEIHARFRRGARSYQVICRDEKTQMI
YQQHQSWLRPVLRSNRVEYCWCNSGRAQCHSVPVKSCSEPRCFNGGTCQ
QALYFSDFVCQCPEGFAGKCCEIDTRATCYEDQGISYRGTWSTAESGAE
CTNWNSSALAQKPYSGRRPDAIRLGLGNHNYCRNPDRDSKPWCYVFKAG
KYSSEFCSTPACSEGNSDCYFGNGSAYRGTHSLTESGASCLPWNSMILI
GKVYTAQNPSAQALGLGKHNYCRNPDGDAKPWCHVLKNRRLTWEYCDVP
SCSTCGLRQYSQPQFRIKGGLFADIASHPWQAAIFAKHRRSPGERFLCG
GILISSCWILSAAHCFQERFPPHHLTVILGRTYRVVPGEEEQKFEVEKY
IVHKEFDDDTYDNDIALLQLKSDSSRCAQESSVVRTVCLPPADLQLPDW
TECELSGYGKHEALSPFYSERLKEAHVRLYPSSRCTSQHLLNRTVTDNM
LCAGDTRSGGPQANLHDACQGDSGGPLVCLNDGRMTLVGIISWGLGCGQ
KDVPGVYTKVTNYLDWIRDNMRP

“Treating” or “treatment” or “alleviation”—As used herein, the terms “treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for the targeted pathologic condition or disorder if, after receiving a therapeutic amount of a protein according to the present invention, the subject or mammal shows observable and/or measurable improvement in one or more of the following: reduction in the number of pathogenic cells; reduction in the percent of total cells that are pathogenic; and/or relief to some extent, of one or more of the symptoms associated with the specific disease or condition; reduced morbidity and mortality, and/or improvement in quality of life issues. The above parameters for assessing successful treatment and improvement in the disease are readily measurable by routine procedures familiar to a physician.

“Variable region” or “variable domain”—As used herein, the term “variable” refers to the fact that certain regions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1 (λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ), consisting of 6 residues) in the VL domain (Morea et al., Methods 20:267-279 (2000)). The first, second and third hypervariable loops of the VKappa light chain domain are referred to herein as L1 (K), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (HI, consisting of 7, 8 or 9 residues), 52-56 (H2, consisting of 3 or 4 residues) and 91-105 (H3, highly variable in length) in the VH domain (Morea et al., Methods 20:267-279 (2000)). Unless otherwise indicated, the terms L1, L2 and L3 respectively refer to the first, second and third hypervariable loops of a VL domain, and encompass hypervariable loops obtained from both VKappa and VLambda isotypes. The terms H1, H2 and H3 respectively refer to the first, second and third hypervariable loops of the VH domain, and encompass hypervariable loops obtained from any of the known heavy chain isotypes, including [gamma], [epsilon], [delta], [alpha] or [mu]. The hypervariable loops L1, L2, L3, H1, H2 and H3 may each comprise part of a “complementarity determining region” or “CDR”, as defined hereinabove.

“Valency”—As used herein, the term “valency” refers to the number of potential target binding sites in a protein. Each target binding site specifically binds one target molecule or specific site on a target molecule. When a protein comprises more than one target binding site, each target binding site may specifically bind the same or different molecules (e.g., may bind to different ligands or different antigens, or different epitopes on the same antigen). The subject binding molecules preferably have at least one binding site specific for a human GPVI molecule. Preferably, the proteins provided herein are monovalent.

### DETAILED DESCRIPTION

An object of the present invention is an inhibitor of the Glycoprotein VI (GPVI) signaling pathway for treating or for use in treating a GPVI-related condition in a subject in need thereof, wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

As used herein, the term “inhibit” means that the inhibitor for use in the present invention is capable of blocking, reducing, preventing or neutralizing GPVI interaction with its ligands, GPVI signaling and/or the activation of molecules from the GPVI signaling pathway.

Examples of ligands of GPVI include, but are not limited to, collagen, fibrin, fibronectin, vitronectin and laminins.

In one embodiment, the inhibitor of GPVI signaling pathway acts by inhibiting the expression of GPVI and/or its ligand.

In one embodiment, the term “inhibit” refers to a decreased expression level as compared to a reference expression level, such as, for example, a level inferior or equal to 90%, 80%, 70%, 60%, 50%, 40%, 30%, 20%, 10%, or less of the reference expression level. In one embodiment, the inhibition of expression of GPVI and/or its ligand is measured after contacting a cell with a compound tested for its impact on expression, and the reference expression level correspond to an expression level measured in a cell not contacted with said compound.

Methods to determine the level of gene expression are well-known to the skilled artisan, and include, without limitation, determining the transcriptome, and/or the proteome.

In vitro methods for assessing the transcription level of a gene are well known in the prior art. Examples of such methods include, but are not limited to, RT-PCR, RT-qPCR, Northern Blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”) and the like.

In vitro methods for assessing the translation level of a gene are well-known in the art. Examples of such methods include, but are not limited to, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), flow cytometry (FACS) and the like.

Examples of inhibitors of the gene expression of GPVI and/or its ligand include, without limitation, small inhibitory RNAs (siRNA), micro RNAs (miRNA) short hairpin RNAs (shRNA), oligonucleotide antisense (including, without limitation, antisense RNA or DNA molecules), ribozymes, aptamers, morpholinos, and engineered nucleases.

In one embodiment, the inhibitor for use in the present invention inhibits the binding of GPVI to a ligand thereof (such as, for example, collagen, fibrin or any other GPVI ligand capable of inducing downstream signaling and platelet activation).

In one embodiment, the inhibitor for use in the present invention binds a ligand of GPVI, preferably human GPVI (hGPVI).

In one embodiment, the inhibitor for use in the present invention acts by occupying the GPVI binding site or a portion thereof of the ligand, thereby making the ligand inaccessible to GPVI, so that its normal biological activity is prevented or reduced.

In one embodiment the inhibitor for use in the present invention binds a ligand of GPVI selected from the group comprising collagen, fibrin, fibronectin, vitronectin and laminins.

In one embodiment, the inhibitor for use in the present invention binds Glycoprotein VI (GPVI), preferably hGPVI.

In one embodiment, the inhibitor for use in the present invention may be a competitive inhibitor, a non-competitive inhibitor, or an uncompetitive inhibitor.

In one embodiment, the inhibitor for use in the present invention is a competitive inhibitor and acts by occupying the ligand binding site or a portion thereof of the receptor (e.g. hGPVI), thereby making the receptor inaccessible to its natural ligand, so that its normal biological activity is prevented or reduced.

In one embodiment, the inhibitor for use in the present invention is a non-competitive inhibitor that reduces the activity of a receptor (e.g. hGPVI) and that binds equally well to the receptor whether or not it has already bound its ligand.

In one embodiment, the inhibitor is an uncompetitive inhibitor that binds only to the complex formed between the receptor (e.g. hGPVI) and its ligand, acting typically in reactions with two or more substrates or products.

Examples of inhibitors of the GPVI signaling pathway include, without limitation, proteins, peptides, polypeptides and small organic molecules.

In one embodiment the inhibitor of the GPVI signaling pathway is an isolated protein, preferably an isolated humanized protein, binding to hGPVI.

Thus, an object of the present invention is an isolated humanized protein binding to hGPVI for treating or for use in treating a GPVI-related condition in a subject in need thereof, wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

In one embodiment, the isolated protein is purified.

In one embodiment, the protein binds to the extracellular domain of GPVI.

In one embodiment, the protein binds to the Ig-like C2-type domain 2 (D2) of human GPVI.

In one embodiment, the isolated protein binds to a conformational epitope.

In one embodiment, the isolated protein binding to human GPVI for use in the present invention has a K_{D} for binding to human GPVI, preferably soluble human GPVI, less than or equal to 15 nM, preferably less than or equal to 10 nM and more preferably less than or equal to 5 nM.

In one embodiment, the isolated protein has a k_{off} for binding to human GPVI of less than or equal to about 8.10⁻⁴ sec⁻¹, preferably less than or equal to about 6.10⁻⁴ sec⁻¹, and more preferably less than or equal to about 4.10⁻⁴ sec⁻¹.

In one embodiment, the isolated protein has a kₒₙ for binding to human GPVI of at least about 5.10⁴ M⁻¹ sec⁻¹, preferably at least about 5.5.10⁴ M⁻¹ sec⁻¹, and more preferably at least about 5.9.10⁴ M⁻¹ sec⁻¹ and more preferably at least about 6.10⁴ sec⁻¹.

In one embodiment, the K_{D} may be determined by Surface plasmon resonance (SPR, BIAcore), using immobilized soluble GPVI at a dose ranging from about 700 to about 1600 resonance units (RU) (corresponding to about 8.5 to about 11.3 fmol/mm²), preferably from about 850 to about 1200 RU, more preferably from about 950 to about 1075 RU and/or using PBS pH 7.4 as running buffer, and/or using BIAevaluation version 3.0 software for analyzing data. In one embodiment, soluble GPVI corresponds to the extracellular domain of GPVI fused at its C-terminus via a linker (such as, for example, via a Gly-Gly-Arg linker) to a hFc sequence. This soluble GPVI may be referred to as soluble GPVI-Fc.

Methods for determining the affinity of a protein for a ligand are well known in the art.

An example of a method for determining the affinity of a protein for soluble GPVI is shown below:

Binding of the protein to soluble human GPVI is analyzed with surface plasmon resonance using a BIAcore 2000 system (Uppsala, Sweden).

Soluble GPVI-Fc is immobilized at a dose ranging from about 700 to about 1600 RU (corresponding to about 8.5 to about 11.3 fmol/mm²), preferably from about 850 to about 1200 RU, more preferably from about 950 to about 1075 RU, and even more preferably from about 960 to about 1071 RU onto a Carboxy-Methyl Dextran CM5 sensor chip using the amine coupling method (Wizard procedure). The protein is then passed over the immobilized GPVI-Fc in PBS pH 7.4 (10 mM phosphate, 138 mM NaCl, 2.7 mM KCl, pH 7.42 at 25.4° C.) at a flow rate of 20 μL/min at 25° C. Kinetic constants (kₒₙ, k_{off}) and affinity are determined using BIAevaluation version 3.0 software, by fitting data to binding model. PBS pH 7.4 is the running buffer.

In an embodiment, the protein for use in the present invention is an antibody molecule or a fragment thereof selected from the group consisting of a whole antibody, a single chain antibody, a dimeric single chain antibody, a Fv, a Fab, a F(ab)′₂, a defucosylated antibody, a bi-specific antibody, a diabody, a triabody and a tetrabody.

In another embodiment, the protein for use in the present invention is an antibody fragment selected from the group consisting of a unibody, a domain antibody, and a nanobody.

In one embodiment, the protein for use in the present invention is an antibody molecule or an antibody fragment selected from the group consisting of a whole antibody, a single chain antibody, a Fv, a Fab, a unibody, a domain antibody, and a nanobody.

In another embodiment, the protein for use in the present invention is an antibody mimetic selected from the group consisting of an affibody, an affilin, an affitin, an adnectin, an atrimer, an evasin, a DARPin, an anticalin, an avimer, a fynomer, a versabody and a duocalin.

A domain antibody is well known in the art and refers to the smallest functional binding units of antibodies, corresponding to the variable regions of either the heavy or light chains of antibodies.

A nanobody is well known in the art and refers to an antibody-derived therapeutic protein that contains the unique structural and functional properties of naturally-occurring heavy chain antibodies. These heavy chain antibodies may contain a single variable domain (VHH) and two constant domains (CH2 and CH3).

A unibody is well known in the art and refers to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

An affibody is well known in the art and refers to affinity proteins based on a 58 amino acid residue protein domain, derived from one of the IgG binding domain of staphylococcal protein A.

DARPins (Designed Ankyrin Repeat Proteins) are well known in the art and refer to an antibody mimetic DRP (designed repeat protein) technology developed to exploit the binding abilities of non-antibody proteins.

Anticalins are well known in the art and refer to another antibody mimetic technology, wherein the binding specificity is derived from lipocalins. Anticalins may also be formatted as dual targeting protein, called Duocalins.

Avimers are well known in the art and refer to another antibody mimetic technology.

Versabodies are well known in the art and refer to another antibody mimetic technology. They are small proteins of 3-5 kDa with >15% cysteines, which form a high disulfide density scaffold, replacing the hydrophobic core the typical proteins have.

In one embodiment, the protein for use in the present invention is monovalent, and is preferably selected from a whole monovalent antibody, a humanized monovalent antibody, a single chain antibody, a Fv, a Fab, a unibody, a domain antibody, and a nanobody; or a monomeric antibody mimetic selected from the group consisting of an affibody, an affilin, an affitin, an adnectin, an atrimer, an evasin, a DARPin, an anticalin, an avimer, a fynomer, and a versabody.

In another embodiment, the protein for use in the present invention is an immunoconjugate comprising an antibody or fragment thereof conjugated to a therapeutic agent.

In another embodiment, the protein for use in the present invention is a conjugate comprising the protein conjugated to an imaging agent. Said protein could be used, for example, for imaging applications.

In an embodiment, the protein for use in the present invention is a monoclonal antibody or a fragment thereof.

In another embodiment, the protein for use in the present invention is a polyclonal antibody or a fragment thereof.

Another object of the invention is thus an anti-hGPVI antibody or a fragment thereof for treating, or for use in treating, a GPVI-related condition in a subject in need thereof, wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

In one embodiment, the variable region of the heavy chain of the anti-hGPVI antibody or fragment thereof for use in the present invention comprises at least one of the followings CDRs:

VH-CDR1:
(SEQ ID NO: 1)
GYTFTSYNMH;
VH-CDR2:
(SEQ ID NO: 2)
GIYPGNGDTSYNQKFQG;
and
VH-CDR3:
(SEQ ID NO: 3)
GTVVGDWYFDV.

CDR numbering and definition are according to the Kabat/Chothia definition.

In one embodiment, the variable region of the light chain of the anti-hGPVI antibody or fragment thereof for use in the present invention comprises at least one of the followings CDRs:

VL-CDR1:
(SEQ ID NO: 4)
RSSQSLENSNGNTYLN;
VL-CDR2:
(SEQ ID NO: 5)
RVSNRFS;
and
VL-CDR3:
(SEQ ID NO: 6)
LQLTHVPWT.

CDR numbering and definition are according to the Kabat/Chothia definition.

In one embodiment, the anti-hGPVI antibody or fragment thereof for use in the present invention comprises:
in the heavy chain, at least one of the following CDR: GYTFTSYNMH (SEQ ID NO: 1), GIYPGNGDTSYNQKFQG (SEQ ID NO: 2) and GTVVGDWYFDV (SEQ ID NO: 3); and/or
in the light chain, at least one of the following CDR: RSSQSLENSNGNTYLN (SEQ ID NO: 4), RVSNRFS (SEQ ID NO: 5), and LQLTHVPWT (SEQ ID NO: 6).

In another embodiment of the invention, the anti-hGPVI antibody or fragment thereof for use in the present invention comprises in its heavy chain the 3 CDRs SEQ ID NO: 1, SEQ ID NO: 2 and SEQ ID NO: 3.

In another embodiment of the invention, the anti-hGPVI antibody or fragment thereof for use in the present invention comprises in its light chain the 3 CDRs SEQ ID NO: 4, SEQ ID NO: 5 and SEQ ID NO: 6.

In another embodiment of the invention, the anti-hGPVI antibody or fragment thereof for use in the present invention comprises in its heavy chain the 3 CDRs SEQ ID NO: 1, SEQ ID NO: 2 and SEQ ID NO: 3, and in its light chain the 3 CDRs SEQ ID NO: 4, SEQ ID NO: 5 and SEQ ID NO: 6.

In another embodiment of the invention, the anti-hGPVI antibody or fragment thereof for use in the present invention comprises in its heavy chain the 3 CDRs SEQ ID NO: 1, SEQ ID NO: 2 and SEQ ID NO: 3, and in its light chain the 3 CDRs SEQ ID NO: 4, SEQ ID NO: 5 and SEQ ID NO: 6, optionally wherein one, two, three or more of the amino acids in any of said sequences may be substituted by a different amino acid.

According to the invention, any of the CDRs 1, 2 and 3 of the heavy and light chains may be characterized as having an amino acid sequence that shares at least 60%, 70%, 75%, 80%, 90%, 95%, 96%, 97%, 98%, 99% of identity with the particular CDR or sets of CDRs listed in the corresponding SEQ ID NO.

In another embodiment of the invention, the anti-hGPVI antibody or fragment thereof for use in the present invention is an antibody or fragment thereof having:
(i) the heavy chain CDR 1, 2 and 3 (VH-CDR1, VH-CDR2, VH-CDR3) amino acid sequences as shown in SEQ ID NO: 1, 2 and 3 respectively; and
(ii) the light chain CDR 1, 2 and 3 (VL-CDR1, VL-CDR2, VL-CDR3) amino acid sequences as shown in SEQ ID NO: 4, 5 and 6 respectively;
   optionally wherein one, two, three or more of the amino acids in any of said sequences may be substituted by a different amino acid.

In one embodiment, the anti-hGPVI antibody or fragment thereof for use in the present invention comprises a heavy chain variable region comprising or consisting of the sequence SEQ ID NO: 7.

(SEQ ID NO: 7)
QVQLVQSGAEVKKPGASVKVSCKASGYTFTSYNMHWVRQAPGQGLEWMGG
IYPGNGDTSYNQKFQGRVTMTRDTSTSTVYMELSSLRSEDTAVYYCARGT
VVGDWYFDVWGQGTLVTVSS..

In one embodiment, the anti-hGPVI antibody or fragment thereof for use in the present invention comprises a light chain variable region comprising or consisting of the sequence SEQ ID NO: 8.

(SEQ ID NO: 8)
DIQMTQSPSSLSASVGDRVTITCRSSQSLENSNGNTYLNWYQQKPGKAPK
LLIYRVSNRFSGVPSRFSGSGSGTDFTFTISSLQPEDIATYYCLQLTHVP
WTFGQGTKVEITR..

In one embodiment, the anti-hGPVI antibody or fragment thereof for use in the present invention comprises a light chain variable region comprising or consisting of the sequence SEQ ID NO: 9.

(SEQ ID NO: 9)
DIQMTQSPSSLSASVGDRVTITCSASQSLENSNGNTYLNWYQQKPGKAPK
LLIYRVSNRFSGVPSRFSGSGSGTDFTLTISSLQPEDFATYYCLQLTHVP
WTFGQGTKVEIKR..

In another embodiment of the invention, the anti-hGPVI antibody (ACT017 antibody) or fragment thereof for use in the present invention comprises a heavy chain variable region comprising or consisting of the sequence SEQ ID NO: 7 and the light chain variable region comprising or consisting of the sequence SEQ ID NO: 8.

In another embodiment of the invention, the anti-hGPVI antibody (ACT006 antibody) or fragment thereof for use in the present invention comprises the heavy chain variable region comprising or consisting of the sequence SEQ ID NO: 7 and the light chain variable region comprising or consisting of the sequence SEQ ID NO: 9.

According to the invention, one, two, three or more of the amino acids of the heavy chain or light chain variable regions as described hereinabove may be substituted by a different amino acid.

In another embodiment, an antibody (or a fragment thereof) for use in the present invention comprises heavy and light chain variable regions comprising amino acid sequences that are homologous to the amino acid sequences of the ACT017 antibody described herein, and wherein the antibodies (or fragments thereof) retain the desired functional properties of the protein described in the present invention.

In another embodiment, an antibody (or a fragment thereof) for use in the present invention comprises heavy and light chain variable regions comprising amino acid sequences that are homologous to the amino acid sequences of the ACT006 antibody described herein, and wherein the antibodies (or fragments thereof) retain the desired functional properties of the protein described in the present invention.

According to the invention, the heavy chain variable region encompasses sequences that have 60%, 70%, 75%, 80%, 90%, 95%, 96%, 97%, 98%, 99% or more of identity with SEQ ID NO: 7.

According to the invention, the light chain variable region encompasses sequences that have 60%, 70%, 75%, 80%, 90%, 95%, 96%, 97%, 98%, 99% or more of identity with SEQ ID NO: 8 or SEQ ID NO: 9.

In any of the antibodies or fragments thereof for use in the present invention (e.g. ACT017 or ACT006), the specified variable region and CDR sequences may comprise conservative sequence modifications. Conservative sequence modifications refer to amino acid modifications that do not significantly affect or alter the binding characteristics of the antibody (or fragment thereof) containing the amino acid sequence. Such conservative modifications include amino acid substitutions, additions and deletions. Modifications can be introduced into an antibody (or fragment thereof) for use in the present invention by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody (or fragment thereof) for use in the present invention can be replaced with other amino acid residues from the same side chain family and the altered antibody (or fragment thereof) can be tested for retained function (i.e., the properties set forth herein) using the assays described herein.

In one embodiment, the antibody (or fragment thereof) for use in the present invention binds essentially the same epitope as ACT017 or ACT006 antibodies. In the present invention, an antibody (or fragment thereof) that binds essentially the same epitope as ACT017 or ACT006 antibodies will be referred as an ACT017-like or ACT006-like antibody, respectively.

In one embodiment, the antibody (or fragment thereof) for use in the present invention competes for binding to hGPVI with an antibody as described hereinabove, in particular with an antibody selected from ACT017 and ACT006.

In some embodiments of this invention, anti-hGPVI antibodies or fragments thereof comprising VH and VL domains, or CDRs thereof may comprise CH1 domains and/or CL domains, the amino acid sequence of which is fully or substantially human. Where the GPVI binding protein is an antibody (or fragment thereof) intended for human therapeutic use, it is typical for the entire constant region of the antibody, or at least a part thereof, to have a fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence. Advantageously, the CH1 domain, hinge region, CH2 domain, CH3 domain and CL domain (and CH4 domain if present) may all have a fully or substantially human amino acid sequence. In the context of the constant region of a humanized or chimeric antibody, or an antibody fragment, the term “substantially human” refers to an amino acid sequence identity of at least 70%, or at least 80%, or at least 90%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates the use of proteins comprising constant domains of “human” sequence which have been altered, by one or more amino acid additions, deletions or substitutions with respect to the human sequence, excepting those embodiments where the presence of a “fully human” hinge region is expressly required. The presence of a “fully human” hinge region in the anti-hGPVI antibodies for use in the present invention may be beneficial both to minimize immunogenicity and to optimize stability of the antibody. It is considered that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody, it may be desirable to modify the antibody with respect to its binding properties to Fc receptors, for example to modulate effector function. For example, cysteine residue(s) may be introduced in the Fc region, thereby allowing interchain disulfide bond formation in this region. The homodimeric antibody thus generated may have improved effector function. See Caron et al., J. Exp. Med. 176:1191-1195 (1992) and Shopes, B. J. Immunol. 148:2918-2922 (1992).

Thus, in one embodiment, the anti-hGPVI antibody or fragment thereof is humanized or chimeric.

In one embodiment, the heavy chain variable region of sequence SEQ ID NO: 7 is encoded by SEQ ID NO: 10:

(SEQ ID NO: 10)
CAGGTTCAGCTGGTTCAGTCAGGGGCTGAGGTGAAGAAGCCTGGAGCCTC
AGTGAAGGTGTCCTGCAAGGCTTCTGGCTACACATTTACCAGTTACAATA
TGCACTGGGTAAGACAGGCTCCTGGACAGGGCCTGGAATGGATGGGAGGT
ATTTATCCAGGAAATGGTGATACTTCCTACAATCAGAAGTTCCAGGGCCG
AGTTACTATGACTCGGGACACTTCCACCTCTACAGTGTACATGGAGCTCA
GCAGCCTGAGATCTGAGGACACCGCGGTCTATTACTGTGCAAGAGGCACC
GTGGTCGGCGACTGGTACTTCGATGTGTGGGGCCAAGGCACCCTGGTCAC
CGTGAGCAGT..

In one embodiment, the light chain variable region of sequence SEQ ID NO: 8 is encoded by SEQ ID NO: 11:

(SEQ ID NO: 11)
GACATCCAGATGACCCAGAGCCCAAGCAGCCTGAGCGCCAGCGTGGGTGA
CAGAGTGACCATCACCTGTAGAAGTAGTCAGAGCCTTGAGAACAGCAACG
GAAACACCTACCTGAATTGGTACCAGCAGAAGCCAGGTAAGGCTCCAAAG
CTGCTGATCTACAGAGTTTCCAACCGATTCTCTGGTGTGCCAAGCAGATT
CAGCGGTAGCGGTAGCGGTACCGACTTCACCTTCACCATCAGCAGCCTCC
AGCCAGAGGACATCGCCACCTACTACTGCCTCCAGCTGACTCATGTCCCA
TGGACCTTCGGTCAGGGCACCAAGGTGGAGATCACCCGG..

In one embodiment, the light chain variable region of sequence SEQ ID NO: 9 is encoded by SEQ ID NO: 12.

(SEQ ID NO: 12)
GACATCCAGATGACCCAGAGCCCAAGCAGCCTGAGCGCCAGCGTGGGTGA
CAGAGTGACCATCACCTGTAGTGCCAGTCAGAGCCTTGAGAACAGCAACG
GAAACACCTACCTGAATTGGTACCAGCAGAAGCCAGGTAAGGCTCCAAAG
CTGCTGATCTACAGAGTTTCCAACCGATTCTCTGGTGTGCCAAGCAGATT
CAGCGGTAGCGGTAGCGGTACCGACTTCACCCTCACCATCAGCAGCCTCC
AGCCAGAGGACTTCGCCACCTACTACTGCCTCCAGCTGACTCATGTCCCA
TGGACCTTCGGTCAGGGCACCAAGGTGGAGATCAAACGC..

In one embodiment, the nucleic sequences encoding the anti-hGPVI antibody or fragment thereof for use in the present invention are comprised in an expression vector. In one embodiment, the expression vector comprises at least one of SEQ ID NO: 10, SEQ ID NO: 11 and SEQ ID NO: 12 or any sequence having a nucleic acid sequence that shares at least 60%, 70%, 75%, 80%, 90%, 95%, 96%, 97%, 98%, 99% of identity with said SEQ ID NO: 10-12.

In one embodiment, the expression vector comprises SEQ ID NO: 10 and SEQ ID NO: 11 or any sequence having a nucleic acid sequence that shares at least 60%, 70%, 75%, 80%, 90%, 95%, 96%, 97%, 98%, 99% of identity with said SEQ ID NO: 10-11.

In one embodiment, the expression vector comprises SEQ ID NO: 10 and SEQ ID NO: 12 or any sequence having a nucleic acid sequence that shares at least 60%, 70%, 75%, 80%, 90%, 95%, 96%, 97%, 98%, 99% of identity with said SEQ ID NO: 10, 12.

In one embodiment, the vector comprises the sequence SEQ ID NO: 10 and a sequence encoding a constant region of a heavy chain. A non-limiting example of a sequence encoding a constant region of a heavy chain is SEQ ID NO: 14.

(SEQ ID NO: 14)
GCCTCCACCAAGGGTCCCTCAGTCTTCCCACTGGCACCCTCCTCCAAGAG
CACCTCTGGTGGCACAGCTGCCCTGGGCTGCCTGGTCAAGGACTACTTCC
CAGAACCAGTGACTGTGTCATGGAACTCAGGCGCCCTGACCAGCGGCGTG
CACACCTTCCCTGCTGTCTTGCAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAAAGTCGAGCCT
AAGTCATGCGACAAGACTCAC..

In one embodiment, the vector comprises the sequence SEQ ID NO: 11 or SEQ ID NO: 12 and a sequence encoding a constant region of a light chain. A non-limiting example of a sequence encoding a constant region of a light chain is SEQ ID NO: 15.

(SEQ ID NO: 15)
ACTGTGGCTGCACCAAGTGTGTTCATCTTCCCACCTAGCGATGAGCAGTT
GAAATCTGGAACTGCCTCTGTCGTGTGCCTCCTGAACAACTTCTACCCAC
GGGAGGCCAAGGTACAGTGGAAGGTGGATAACGCCCTCCAATCCGGTAAC
TCCCAGGAGAGTGTCACAGAGCAAGATAGCAAGGACAGCACCTACAGCCT
CAGCAGCACCCTGACTCTGAGCAAAGCAGACTACGAGAAGCACAAGGTCT
ACGCCTGCGAAGTCACCCATCAGGGCCTGAGTTCCCCTGTCACAAAGAGC
TTCAACCGGGGAGAGTGT..

In one embodiment, the vector comprises a sequence encoding a signal peptide. Non-limiting examples of signal peptides sequences include, but are not limited to, SEQ ID NO: 16 and SEQ ID NO: 17.

(SEQ ID NO: 16)
ATGGATATGCGTGTACCAGCTCAACTACTTGGACTTCTATTGCTTTGGCT
TCGTGGTGCTAGATGT..
(SEQ ID NO: 17)
ATGGACTGGACTTGGAGAATCCTATTCTTGGTTGCTGCAGCTACAGGTGC
TCATTCA..

In one embodiment, the vector comprises SEQ ID NO: 10, and a sequence encoding a constant region of a heavy chain (such as, for example, SEQ ID NO: 14), and a signal peptide sequence. An example of such a vector is a vector comprising SEQ ID NO: 18. SEQ ID NO: 18 further comprises cloning sites.

(SEQ ID NO: 18)
GCGGCCGCCACCATGGACTGGACTTGGAGAATCCTATTCTTGGTTGCTGC
AGCTACAGGTGCTCATTCACAGGTTCAGCTGGTTCAGTCAGGGGCTGAGG
TGAAGAAGCCTGGAGCCTCAGTGAAGGTGTCCTGCAAGGCTTCTGGCTAC
ACATTTACCAGTTACAATATGCACTGGGTAAGACAGGCTCCTGGACAGGG
CCTGGAATGGATGGGAGGTATTTATCCAGGAAATGGTGATACTTCCTACA
ATCAGAAGTTCCAGGGCCGAGTTACTATGACTCGGGACACTTCCACCTCT
ACAGTGTACATGGAGCTCAGCAGCCTGAGATCTGAGGACACCGCGGTCTA
TTACTGTGCAAGAGGCACCGTGGTCGGCGACTGGTACTTCGATGTGTGGG
GCCAAGGCACCCTGGTCACCGTGAGCAGTGCCTCCACCAAGGGTCCCTCA
GTCTTCCCACTGGCACCCTCCTCCAAGAGCACCTCTGGTGGCACAGCTGC
CCTGGGCTGCCTGGTCAAGGACTACTTCCCAGAACCAGTGACTGTGTCAT
GGAACTCAGGCGCCCTGACCAGCGGCGTGCACACCTTCCCTGCTGTCTTG
CAGTCCTCAGGACTCTACTCCCTCAGCAGCGTGGTGACCGTGCCCTCCAG
CAGCTTGGGCACCCAGACCTACATCTGCAACGTGAATCACAAGCCCAGCA
ACACCAAGGTGGACAAGAAAGTCGAGCCTAAGTCATGCGACAAGACTCAC
TGATGAGGATCC..

In one embodiment, the vector comprises SEQ ID NO: 11, and a sequence encoding a constant region of a light chain (such as, for example, SEQ ID NO: 15), and a signal peptide sequence. An example of such a vector is a vector comprising SEQ ID NO: 19. SEQ ID NO: 19 further comprises cloning sites.

(SEQ ID NO: 19)
GACGTCACCATGGATATGCGTGTACCAGCTCAACTACTTGGACTTCTATT
GCTTTGGCTTCGTGGTGCTAGATGTGACATCCAGATGACCCAGAGCCCAA
GCAGCCTGAGCGCCAGCGTGGGTGACAGAGTGACCATCACCTGTAGAAGT
AGTCAGAGCCTTGAGAACAGCAACGGAAACACCTACCTGAATTGGTACCA
GCAGAAGCCAGGTAAGGCTCCAAAGCTGCTGATCTACAGAGTTTCCAACC
GATTCTCTGGTGTGCCAAGCAGATTCAGCGGTAGCGGTAGCGGTACCGAC
TTCACCTTCACCATCAGCAGCCTCCAGCCAGAGGACATCGCCACCTACTA
CTGCCTCCAGCTGACTCATGTCCCATGGACCTTCGGTCAGGGCACCAAGG
TGGAGATCACCCGGACTGTGGCTGCACCAAGTGTGTTCATCTTCCCACCT
AGCGATGAGCAGTTGAAATCTGGAACTGCCTCTGTCGTGTGCCTCCTGAA
CAACTTCTACCCACGGGAGGCCAAGGTACAGTGGAAGGTGGATAACGCCC
TCCAATCCGGTAACTCCCAGGAGAGTGTCACAGAGCAAGATAGCAAGGAC
AGCACCTACAGCCTCAGCAGCACCCTGACTCTGAGCAAAGCAGACTACGA
GAAGCACAAGGTCTACGCCTGCGAAGTCACCCATCAGGGCCTGAGTTCCC
CTGTCACAAAGAGCTTCAACCGGGGAGAGTGTTGATGATATC..

In one embodiment, the vector comprises SEQ ID NO: 12, and a sequence encoding a constant region of a light chain (such as, for example, SEQ ID NO: 15), and a signal peptide sequence. An example of such a vector is a vector comprising SEQ ID NO: 20. SEQ ID NO: 20 further comprises cloning sites.

(SEQ ID NO: 20)
GACGTCACCATGGATATGCGTGTACCAGCTCAACTACTTGGACTTCTATT
GCTTTGGCTTCGTGGTGCTAGATGTGACATCCAGATGACCCAGAGCCCAA
GCAGCCTGAGCGCCAGCGTGGGTGACAGAGTGACCATCACCTGTAGTGCC
AGTCAGAGCCTTGAGAACAGCAACGGAAACACCTACCTGAATTGGTACCA
GCAGAAGCCAGGTAAGGCTCCAAAGCTGCTGATCTACAGAGTTTCCAACC
GATTCTCTGGTGTGCCAAGCAGATTCAGCGGTAGCGGTAGCGGTACCGAC
TTCACCCTCACCATCAGCAGCCTCCAGCCAGAGGACTTCGCCACCTACTA
CTGCCTCCAGCTGACTCATGTCCCATGGACCTTCGGTCAGGGCACCAAGG
TGGAGATCAAACGCACTGTGGCTGCACCAAGTGTGTTCATCTTCCCACCT
AGCGATGAGCAGTTGAAATCTGGAACTGCCTCTGTCGTGTGCCTCCTGAA
CAACTTCTACCCACGGGAGGCCAAGGTACAGTGGAAGGTGGATAACGCCC
TCCAATCCGGTAACTCCCAGGAGAGTGTCACAGAGCAAGATAGCAAGGAC
AGCACCTACAGCCTCAGCAGCACCCTGACTCTGAGCAAAGCAGACTACGA
GAAGCACAAGGTCTACGCCTGCGAAGTCACCCATCAGGGCCTGAGTTCCC
CTGTCACAAAGAGCTTCAACCGGGGAGAGTGTTGATGATATC..

In one embodiment, the vector as described hereinabove is comprised in an isolated host cell. Said host cell may be used for the recombinant production of the antibodies (or fragments thereof) for use in the present invention. In an embodiment, host cells may be prokaryote, yeast, or eukaryote cells preferably mammalian cells, such as, for example: monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 85110503); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art. It should be noted that the term “host cell” generally refers to a cultured cell line. Whole human beings into which an expression vector encoding an antigen binding protein for use according to the invention has been introduced are explicitly excluded from the definition of a “host cell”.

Methods for producing an anti-hGPVI antibody or fragment thereof for use in the present invention are well known in the art. Examples of suitable methods comprise culturing host cells containing the isolated polynucleotide sequence encoding the anti-hGPVI antibody or fragment thereof for use in the present invention under conditions suitable for expression of the anti-hGPVI antibody or fragment thereof, and recovering the expressed anti-hGPVI antibody or fragment thereof. This recombinant process can be used for large scale production of anti-hGPVI antibodies or fragment thereof for use in the present invention, including monoclonal antibodies intended for in vivo therapeutic uses. These processes are available in the art and will be known by the skilled person.

In one embodiment, the protein for use in the present invention may be purified by chromatography, preferably by affinity chromatography, more preferably by affinity chromatography on protein L agarose.

Therefore, in one embodiment, the protein for use in the present invention comprises a domain for binding protein L (PpL). Methods for transferring PpL-binding activity onto proteins of the invention are described in Muzard et al., Analytical Biochemistry 388, 331-338, 2009 and in Lakhrif et al., MAbs. 2016; 8 (2): 379-88, which are incorporated herein by reference.

Fragments and derivatives of antibodies for use in the present invention (which are encompassed by the term “antibody” or “antibodies” as used in this application, unless otherwise stated or clearly contradicted by context), preferably a ACT017-like or ACT006-like antibody, can be produced by techniques that are known in the art.

“Fragments” comprise a portion of the intact antibody, generally the antigen binding site or variable region. Examples of antibody fragments include Fab, Fab′, Fab′-SH, F (ab′)₂, and Fv fragments; diabodies; any antibody fragment that is a protein having a primary structure consisting of one uninterrupted sequence of contiguous amino acid residues (referred to herein as a “single-chain antibody fragment” or “single chain protein”), including without limitation (1) single-chain Fv molecules (2) single chain proteins containing only one light chain variable domain, or a fragment thereof that contains the three CDRs of the light chain variable domain, without an associated heavy chain moiety and (3) single chain proteins containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods. For instance, Fab or F(ab′)2 fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al., Cytokines 16 (3): 106-119 (2001) and Delgado et al., Br. J. Cancer 73 (2): 175-182 (1996), the disclosures of which are incorporated herein by reference.

Alternatively, the DNA encoding an antibody for use in the present invention, preferably an ACT017-like or ACT006-like antibody, may be modified so as to encode a fragment. The modified DNA is then inserted into an expression vector and used to transform or transfect an appropriate cell, which then expresses the desired fragment.

Another object of the invention is a composition for treating or for use in treating a GPVI-related condition in a subject in need thereof, wherein said composition comprises at least one inhibitor of the GPVI signaling pathway, in particular at least one isolated humanized protein binding to human Glycoprotein VI (hGPVI), as described here above, and wherein said subject presents at least one of the following conditions:
(i). said subject is affected with a moderate to severe GPVI-related condition,
(ii). said subject is of 65 years of age older, and/or
(iii). said subject is undergoing or has undergone thrombectomy and thrombolysis,
   preferably wherein said GPVI-related condition is a stroke.

Another object of the invention is a pharmaceutical composition for treating or for use in treating a GPVI-related condition in a subject in need thereof, wherein said pharmaceutical composition comprises at least one inhibitor of the GPVI signaling pathway, in particular at least one isolated humanized protein binding to human Glycoprotein VI (hGPVI), as described here above and at least one pharmaceutically acceptable excipient, and wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

Pharmaceutically acceptable excipients that may be used in these compositions include, but are not limited to, ion exchangers, alumina, aluminum stearate, lecithin, serum proteins, such as human serum albumin, buffer substances such as acetates, citrates, phosphates, glycine, sorbic acid, potassium sorbate, sugars such as glucose, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pH of the pharmaceutical composition is comprised between 4 to 6 and preferably is of about 5.0.

Another object of the invention is a medicament for treating or for use in treating a GPVI-related condition in a subject in need thereof, wherein said medicament comprises at least one inhibitor of GPVI signaling pathway, in particular at least one isolated humanized protein binding to human Glycoprotein VI (hGPVI), as described here above, and wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

Another object of the invention is the use of at least one inhibitor of GPVI signaling pathway, in particular at least one isolated humanized protein binding to human Glycoprotein VI (hGPVI), as described here above in the manufacture of a medicament for treating a GPVI-related condition in a subject in need thereof, and wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

In one embodiment, the composition, pharmaceutical composition or medicament for use in the present invention further comprises another agent useful for treating a GPVI-related condition, such as, for example a thrombolytic agent.

As used herein, thrombolytic agents are agents capable of dissolving a clot (or thrombus) formed in blood vessels. Examples of thrombolytic agents, include, without limitation, anistreplase (e.g. Eminase); tissue-type plasminogen activator (t-PA) and modified forms of t-PA; streptokinase (e.g. Streptase); urokinase, pro-urokinase and modified forms thereof (e.g. Abbokinase, Kinlytic, TS-01); small molecule plasminogen activator (e.g. TMS-007; SMTP-7; BIIB131); and N-Acetyl Cysteine (NAC).

In one embodiment, the thrombolytic agent is anistreplase (also known as anisoylated plasminogen streptokinase activator complex (APSAC)) (e.g. Eminase). In one embodiment, the thrombolytic agent is streptokinase (e.g. Streptase).

In one embodiment, the thrombolytic agent is urokinase or pro-urokinase (e.g. Abbokinase, Kinlytic). In one embodiment, the thrombolytic agent is a modified form of pro-urokinase (e.g. TS-01).

In one embodiment, the thrombolytic agent is a small molecule plasminogen activator (e.g. TMS-007).

In one embodiment, the thrombolytic agent is N-Acetyl Cysteine (NAC).

In one embodiment, the thrombolytic agent is t-PA, including native t-PA and recombinant t-PA, as well as modified forms of t-PA that retain the enzymatic and/or fibrinolytic activity of native t-PA. The enzymatic activity of t-PA, such as a modified form of t-PA, may be measured by assessing the ability of the molecule to convert plasminogen to plasmin. The fibrinolytic activity of t-PA, such as a modified form of t-PA, may be determined by any in vitro clot lysis test known in the art.

Native t-PA include t-PA whose sequences are naturally found in wild-type organisms, such as mammal t-PA, preferably human t-PA. Native t-PA may be obtained by methods well known by the skilled artisan in the art, such as, for example, isolation from natural sources or production by recombinant or synthetic means. In one embodiment, the native t-PA is the human t-PA of sequence SEQ ID NO: 25 or SEQ ID NO: 28.

Recombinant t-PA have been described in the art and are known by the skilled artisan. Recombinant t-PA may be native t-PA as described hereinabove or modified forms of t-PA as described hereinbelow, produced by recombinant means. Recombinant t-PA may be encoded by recombinant DNA that has been cloned in an expression vector that supports expression of the gene in a host cell. Examples of host cells and production of proteins are provided hereinabove for anti-hGPVI antibodies (or fragments thereof) and can apply for t-PA. Recombinant t-PA are commercially available as, for example, alteplase (Activase® or Actilyse®), reteplase (Retavase) or tenecteplase (TNKase, Metalyse and Elaxi). Alteplase is a recombinant human t-PA produced in Chinese hamster ovary cells, which may have the sequence SEQ ID NO: 29. Reteplase is a recombinant non-glycosylated form of human t-PA produced in Escherichia coli bacteria, having the sequence SEQ ID NO: 30. Tenecteplase is a recombinant t-PA produced in Chinese hamster ovary cells, having the sequence SEQ ID NO: 31.

(SEQ ID NO: 29)
SYQVICRDEKTQMIYQQHQSWLRPVLRSNRVEYCWCNSGRAQCHSVPVKS
CSEPRCFNGGTCQQALYFSDFVCQCPEGFAGKCCEIDTRATCYEDQGISY
RGTWSTAESGAECTNWNSSALAQKPYSGRRPDAIRLGLGNHNYCRNPDRD
SKPWCYVFAGKYSSEFCSTPACSEGNSDCYFGNGSAYRGTHSLTESGASC
LPWNSMILIGKVYTAQNPSAQALGLGKHNYCRNPDGDAKPWCHVLKNRRL
TWEYCDVPSCSTCGLRQYSQPQFRIKGGLFADIASHPWQAAIFAKHRRSP
GERFLCGGILISSCWILSAAHCFQERFPPHHLTVILGRTYRVVPGEEEQK
FEVEKYIVHKEFDDDTYDNDIALLQLKSDSSRCAQESSVVRTVCLPPADL
QLPDWTECELSGYGKHEALSPFYSERLKEAHVRLYPSSRCTSQHLLNRTV
TDNMLCAGDTRSGGPQANLHDACQGDSGGPLVCLNDGRMTLVGIISWGLG
CGQKDVPGVYTKVTNYLDWIRDNMRP
(SEQ ID NO: 30)
SYQGNSDCYFGNGSAYRGTHSLTESGASCLPWNSMILIGKVYTAQNPSAQ
ALGLGKHNYCRNPDGDAKPWCHVLKNRRLTWEYCDVPSCSTCGLRQYSQP
QFRIKGGLFADIASHPWQAAIFAKHRRSPGERFLCGGILISSCWILSAAH
CFQERFPPHHLTVILGRTYRVVPGEEEQKFEVEKYIVHKEFDDDTYDNDI
ALLQLKSDSSRCAQESSVVRTVCLPPADLQLPDWTECELSGYGKHEALSP
FYSERLKEAHVRLYPSSRCTSQHLLNRTVTDNMLCAGDTRSGGPQANLHD
ACQGDSGGPLVCLNDGRMTLVGIISWGLGCGQKDVPGVYTKVTNYLDWIR
DNMRP
(SEQ ID NO: 31)
SYQVICRDEKTQMIYQQHQSWLRPVLRSNRVEYCWCNSGRAQCHSVPVKS
CSEPRCFNGGTCQQALYFSDFVCQCPEGFAGKCCEIDTRATCYEDQGISY
RGNWSTAESGAECTNWQSSALAQKPYSGRRPDAIRLGLGNHNYCRNPDRD
SKPWCYVFKAGKYSSEFCSTPACSEGNSDCYFGNGSAYRGTHSLTESGAS
CLPWNSMILIGKVYTAQNPSAQALGLGKHNYCRNPDGDAKPWCHVLKNRR
LTWEYCDVPSCSTCGLRQYSQPQFRIKGGLFADIASHPWQAAIFAAAAAS
PGERFLCGGILISSCWILSAAHCFQERFPPHHLTVILGRTYRVVPGEEEQ
KFEVEKYIVHKEFDDDTYDNDIALLQLKSDSSRCAQESSVVRTVCLPPAD
LQLPDWTECELSGYGKHEALSPFYSERLKEAHVRLYPSSRCTSQHLLNRT
VTDNMLCAGDTRSGGPQANLHDACQGDSGGPLVCLNDGRMTLVGIISWGL
GCGQKDVPGVYTKVTNYLDWIRDNMRP

In one embodiment, the thrombolytic agent is a recombinant t-PA, preferably alteplase or tenecteplase.

Modified forms of t-PA have been described in the art and is known by the skilled artisan, and include, without limitation, t-PA having deleted or substituted amino acids or domains, modified glycosylation status, and variants conjugated to or fused with other molecules. Examples of modified forms of t-PA were described, for example, hereinabove and in EP0352119, WO93/24635, EP0382174 and WO2013/034710, which are incorporated herein by reference.

In one embodiment, the composition, pharmaceutical composition or medicament for use in the present invention further comprises a mutated t-PA as described in WO2013/034710.

In one embodiment, said mutated t-PA comprises the sequence SEQ ID NO: 25 (corresponding to human wt t-PA mature form) or SEQ ID NO: 26 (corresponding to human wt t-PA first chain of tc-t-PA), preferably consisting of SEQ ID NO: 25 or of the association of SEQ ID NO:26 and SEQ ID NO:27 (corresponding to human wt t-PA second chain of tc-t-PA), or variant thereof having at least 80% identity, wherein said sequence comprises a mutation consisting of the replacement of any amino acid of the Lysine Binding Site of SEQ ID NO: 25 or SEQ ID NO:26 by a hydrophilic amino acid chosen from arginine, aspartic acid, glutamic acid, lysine, asparagine, glutamine, serine, threonine, tyrosine and histidine, preferably by arginine, and/or a mutation consisting of the replacement of arginine in position 275 of SEQ ID NO: 25 or SEQ ID NO:26 by serine.

In one embodiment, said mutated t-PA comprises the sequence SEQ ID NO: 25 (corresponding to human wt t-PA mature form) or SEQ ID NO: 26 (corresponding to human wt t-PA first chain of two chain t-PA), preferably consisting of SEQ ID NO: 25 or of the association of SEQ ID NO:26 and SEQ ID NO:27 (corresponding to human wt t-PA second chain of two chain t-PA), or variant thereof having at least 80% identity, wherein said sequence comprises a mutation consisting of the replacement of tryptophan in position 253 of SEQ ID NO: 25 or SEQ ID NO:26 by a hydrophilic amino acid chosen from arginine, aspartic acid, glutamic acid, lysine, asparagine, glutamine, serine, threonine, tyrosine and histidine, preferably by arginine, and/or a mutation consisting of the replacement of arginine in position 275 of SEQ ID NO: 25 or SEQ ID NO:26 by serine.

In one embodiment, said mutated t-PA further comprises at least one of the following mutations:
the replacement of proline in position 125 of SEQ ID NO:25 or SEQ ID NO:26 by arginine,
the deletion of the Finger domain in the N-terminus and/or the deletion of the EGF-like domain, in SEQ ID NO:25 or SEQ ID NO:26, and/or the replacement of asparagine in position 117 of SEQ ID NO:25 or SEQ ID NO:26 by glutamine,
the replacement of threonine in position 103 of SEQ ID NO:25 or SEQ ID NO:26 by asparagine, and/or the replacement of asparagine in position 117 of SEQ ID NO: 25 or SEQ ID NO:26 by glutamine, and/or the replacement of lysine-histidine-arginine-arginine (KHRR) in positions 296 to 299 of SEQ ID NO:25 by alanine-alanine-alanine-alanine (AAAA),
the replacement of cysteine in position 84 of SEQ ID NO:25 or SEQ ID NO:26 by serine,
the replacement of arginine in position 275 of SEQ ID NO:25 or SEQ ID NO:26 by glutamic acid or glycine, and/or the deletion of the Kringle 1 domain in SEQ ID NO: 25 or SEQ ID NO:26.

Therefore, in one embodiment, the composition, pharmaceutical composition or medicament for use in the present invention comprises at least one inhibitor of GPVI signaling pathway, preferably an isolated humanized protein binding to human Glycoprotein VI (hGPVI) as described here above, more preferably at least one anti-hGPVI antibody as described hereinabove or a fragment thereof, and t-PA (either in a native, recombinant or modified form).

One object of the invention is a kit of part comprising, in a first part, at least one inhibitor of GPVI signaling pathway, in particular at least one isolated humanized protein binding to human Glycoprotein VI (hGPVI), as described hereinabove (preferably at least one isolated anti-hGPVI antibody or a fragment thereof as described hereinabove) and, in a second part, at least one thrombolytic agent, preferably t-PA (either in a native, recombinant or modified form), for use in the treatment of a GPVI-related condition in a subject in need thereof, wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

In one embodiment, the elements of the kit of parts are to be administered concomitantly to the subject. In one embodiment, the elements of the kit of parts are to be administered sequentially to the subject.

In one embodiment, the at least one inhibitor of GPVI signaling pathway, in particular the at least one humanized protein binding to hGPVI, as described hereinabove is to be administered before the thrombolytic agent, preferably t-PA (either in a native, recombinant or modified form). In one embodiment, the at least one inhibitor of GPVI signaling pathway, in particular the at least one humanized protein binding to hGPVI, as described hereinabove is to be administered less than 48 hours, 24 hours, 12 hours, 8 hours, 4 hours, 3 hours, 2.5 hours, 2 hours, 1.5 hours, 1 hour or less, before the administration of the thrombolytic agent, preferably t-PA (either in a native, recombinant or modified form).

In one embodiment, the at least one inhibitor of GPVI signaling pathway, in particular the at least one humanized protein binding to hGPVI, as described hereinabove is to be administered after the thrombolytic agent, preferably t-PA (either in a native, recombinant or modified form).

In one embodiment, the at least one inhibitor of GPVI signaling pathway, in particular the at least one humanized protein binding to hGPVI, as described hereinabove is to be administered less than 48 hours, 24 hours, 12 hours, 8 hours, 4 hours, 3 hours, 2.5 hours, 2 hours, 1.5 hours, 1 hour or less, after the administration of the thrombolytic agent, preferably t-PA (either in a native, recombinant or modified form). In one embodiment, the at least one inhibitor of GPVI signaling pathway, in particular the at least one humanized protein binding to hGPVI, as described hereinabove is to be administered less than 2.5 hours after the administration of the thrombolytic agent, preferably t-PA (either in a native, recombinant or modified form).

In one embodiment, the composition, pharmaceutical composition or medicament for use in the present invention further comprises another agent useful for treating a GPVI-related condition, such as, for example an antiplatelet agent.

In one embodiment, said antiplatelet agent is selected from the group comprising or consisting of irreversible cyclooxygenase inhibitors such as, for example, Aspirin (Acetylsalicylic acid (ASA)), indobufen (Ibustrin) or Triflusal (Disgren); Adenosine diphosphate (ADP) receptor inhibitors such as, for example, Cangrelor (Kengreal), Clopidogrel (Plavix), Prasugrel (Effient), Ticagrelor (Brilinta) or Ticlopidine (Ticlid); Phosphodiesterase inhibitors such as, for example, Cilostazol (Pletaal); Protease-activated receptor-1 (PAR-1) antagonists such as, for example, Vorapaxar (Zontivity); Glycoprotein IIB/IIIA inhibitors such as, for example, Abciximab (ReoPro), Eptifibatide (Integrilin) or Tirofiban (Aggrastat); Prostacycline analogs such as, for example, Iloprost (Ilomedine), Epoprostenol (Veletri) or Treprostinil (Remodulin); Prostacycline receptor inhibitors such as, for example, Selexipag (Uptravi); Adenosine reuptake inhibitors such as, for example, Dipyridamole (Persantine); Thromboxane inhibitors, Thromboxane synthase inhibitors and Thromboxane receptor antagonists such as, for example, Terutroban.

In one embodiment, said antiplatelet agent is an ADP receptor inhibitor, more preferably Clopidogrel. In one embodiment, said antiplatelet agent is an irreversible cyclooxygenase inhibitor, preferably ASA.

In one embodiment, the composition, pharmaceutical composition or medicament for use in the present invention further comprises another agent useful for treating a GPVI-related condition, such as, for example, an anticoagulant agent.

In one embodiment, said anticoagulant agent is selected from the group comprising or consisting of vitamin K antagonists including coumarin derivatives, such as warfarin (Coumadin), Fluindione (Previscan), Phenprocoumone or acenocoumarol; heparin and derivatives thereof including low molecular weight heparin (LMWH), such as, for example Enoxaparin, Nadroparin, Tinzaparin or Dalteparin; synthetic pentasaccharide inhibitors of factor Xa, such as, for example, Fondaparinux, Idraparinux or Idrabiotaparinux; directly acting oral anticoagulants (DOACs) including direct factor Xa inhibitors (e.g., rivaroxaban, apixaban, edoxaban, betrixaban, darexaban, letaxaban, and eribaxaban) and direct thrombin inhibitors (e.g, hirudin, lepirudin, bivalirudin; argatroban and dabigatran); antithrombin protein therapeutics; and other agents such as, for example, Batroxobin; Hementin and Vitamin E.

In one embodiment, the anticoagulant agent is heparin and or a derivative thereof, preferably a low molecular weight heparin, more preferably Enoxaparin or Dalteparin. In one embodiment, the anticoagulant agent is a vitamin K antagonist, preferably a coumarin derivative, more preferably acenocoumarol.

In one embodiment, the anticoagulant agent is an agent targeting the von Willebrand factor, such as, for example, Caplacizumab (Cablivi).

In one embodiment, the anticoagulant agent is an activated protein C and or a derivative thereof, such as, for example, human protein C (Ceprotin), 3K3A-APC.

In one embodiment, the composition, pharmaceutical composition or medicament for use in the present invention further comprises at least one agent, such as, for example, 1, 2 or 3 agents, selected from the group comprising or consisting of a thrombolytic agent, an antiplatelet agent, and an anticoagulant agent.

One object of the invention is a kit of part comprising, in a first part, at least one inhibitor of GPVI signaling pathway, in particular at least one isolated humanized protein binding to human Glycoprotein VI (hGPVI), as described hereinabove (preferably at least one isolated anti-hGPVI antibody or a fragment thereof as described hereinabove) and, in a second part, at least one agent, such as, for example, 1, 2, 3 or 4 agents, selected from the group comprising or consisting of a thrombolytic agent, an antiplatelet agent, and an anticoagulant agent.

In one embodiment, the kit of part comprises, in a first part, at least one inhibitor of GPVI signaling pathway, in particular at least one isolated humanized protein binding to human Glycoprotein VI (hGPVI), as described hereinabove, and, in a second part, an antiplatelet agent. In one embodiment, the kit of part comprises, in a first part, at least one inhibitor of GPVI signaling pathway, in particular at least one isolated humanized protein binding to human Glycoprotein VI (hGPVI), as described hereinabove, and, in a second part, an anticoagulant agent. In one embodiment, the kit of part comprises, in a first part, at least one inhibitor of GPVI signaling pathway, in particular at least one isolated humanized protein binding to human Glycoprotein VI (hGPVI), as described hereinabove, in a second part, an antiplatelet agent and, in a third part, an anticoagulant agent. In one embodiment, the kit of part comprises, in a first part, at least one inhibitor of GPVI signaling pathway, in particular at least one isolated humanized protein binding to human Glycoprotein VI (hGPVI), as described hereinabove, in a second part, an antiplatelet agent and, in a third part, a thrombolytic agent. In one embodiment, the kit of part comprises, in a first part, at least one inhibitor of GPVI signaling pathway, in particular at least one isolated humanized protein binding to human Glycoprotein VI (hGPVI), as described hereinabove, in a second part, an anticoagulant agent and, in a third part, a thrombolytic agent. In one embodiment, the kit of part comprises, in a first part, at least one inhibitor of GPVI signaling pathway, in particular at least one isolated humanized protein binding to human Glycoprotein VI (hGPVI), as described hereinabove, in a second part, an antiplatelet agent, in a third part, an anticoagulant agent and, in a fourth part, a thrombolytic agent.

In one embodiment, the kit of part is for use in the treatment of a GPVI-related condition in a subject in need thereof, wherein said subject presents at least one of the following conditions:
(i). said subject is affected with a moderate to severe GPVI-related condition,
(ii). said subject is of 65 years of age or older, and/or
(iii). said subject is undergoing or has undergone thrombectomy and thrombolysis,
   preferably wherein said GPVI-related condition is a stroke.

In one embodiment, the inhibitor for use in the present invention inhibits GPVI signaling and/or signaling of a ligand of GPVI.

In one embodiment, the inhibitor, in particular the protein, for use in the present invention is an anti-hGPVI antibody or fragment thereof that inhibits GPVI signaling and/or signaling of a ligand of GPVI. As used herein, the term “inhibit” means that the inhibitor, in particular the protein, for use in the present invention is capable of blocking, reducing, preventing or neutralizing GPVI interaction with its ligands, GPVI signaling and/or the activation of molecules from the GPVI signaling pathway.

Examples of ligands of GPVI are provided hereinabove.

In one embodiment, the inhibitor, in particular the protein, for use in the present invention is a neutralizing anti-hGPVI antibody or fragment thereof.

In one embodiment, the inhibitor, in particular the protein, for use in the present invention inhibits the binding of GPVI to a ligand thereof (such as, for example, collagen, fibrin or any other GPVI ligand capable of inducing downstream signaling and platelet activation).

In one embodiment, the inhibitor, in particular the protein, for use in the present invention inhibits and/or prevents platelet activation, secretion and aggregation in response to a ligand of GPVI, such as, for example, collagen. In an embodiment, the inhibitor, in particular the protein, for use in the present invention inhibits and/or prevents platelet adhesion to a ligand of GPVI, such as, for example, collagen.

In one embodiment, the inhibitor, in particular the protein, for use in the present invention inhibits and/or prevents GPVI-dependent thrombin production in response to a ligand of GPVI, such as, for example, fibrin. In an embodiment, the inhibitor, in particular the protein, for use in the present invention inhibits and/or prevent platelet-catalyzed thrombin production in response to collagen and/or to tissue factor.

In one embodiment, the inhibitor, in particular the protein, for use in the present invention inhibits and/or prevents platelet recruitment by a ligand of GPVI (such as, for example, fibrin) via GPVI.

In one embodiment, the inhibitor, in particular the protein, for use in the present invention induces saturation of platelets in whole blood or in platelet rich plasma when present at a concentration ranging from about 1 to about 200 μg/mL, preferably from about 2 to about 100 μg/mL, and more preferably from about 5 to about 50 μg/mL.

In one embodiment, the inhibitor, in particular the protein, for use in the present invention inhibits collagen-induced platelet aggregation when used at a concentration of at least about 15 μg/mL, preferably of at least about 10 μg/mL. Preferably, the inhibitor, in particular the protein, for use in the present invention fully inhibits collagen-induced platelet aggregation when used at such concentrations.

In one embodiment, the IC50 of the inhibitor, in particular the protein, for use in the present invention for inhibiting collagen-induced platelet aggregation ranges from about 0.5 to about 10 μg/mL, preferably from about 1 to about 6 μg/mL, more preferably from about 2 to about 3.2 μg/mL.

In one embodiment, the concentration of the inhibitor, in particular the protein, for use in the present invention reducing by 50% the velocity of collagen-induced platelet aggregation ranges from about 0.5 to about 5 μg/mL, preferably from about 1 to about 3 μg/mL, more preferably of about 2 μg/mL.

In one embodiment, the concentration of the inhibitor, in particular the protein, for use in the present invention reducing the intensity of collagen-induced platelet aggregation ranges from about 0.5 to about 10 μg/mL, preferably from about 1 to about 6 μg/mL, more preferably of about 3.2 μg/mL.

In one embodiment, the inhibitor, in particular the protein, for use in the present invention does not induce depletion of GPVI when administered in vivo, such as, for example, when administered at a dose ranging from 0.01 to 500 mg.

In one embodiment, the inhibitor, in particular the protein, for use in the present invention does not induce a decrease in platelet count, i.e., thrombocytopenia, when administered in vivo, such as, for example, when administered at a dose ranging from 0.01 to 500 mg. In one embodiment, the inhibitor, in particular the protein, for use in the present invention decreases the incidence of the intracranial hemorrhages (ICHs) or prevents the occurrence of ICHs in a subject affected by, preferably diagnosed with a GPVI-related condition.

In one embodiment, said intracranial hemorrhages are symptomatic ICHs. In one embodiment, said intracranial hemorrhages are non-symptomatic ICHs.

It is well known by the skilled artisan in the art that ICHs may be classified by classifications, such as, for example, the Heidelberg classification. According to the Heidelberg classification, the ICH may be classified in classes and/or types described in the Table 1 below.

TABLE 1

| Class | Type | Description |
|---|---|---|
| 1 | | Hemorrhagic transformation of infarcted brain tissue |
| 1a | HI1 | Scattered small petechiae, no mass effect |
| 1b | HI2 | Confluent petechiae, no mass effect |
| 1c | PH1 | Hematoma within infarcted tissue, occupying <30%, no |
| | | substantive mass effect |
| 2 | | Intracerebral hemorrhage within and beyond infarcted |
| | | brain tissue |
| | PH2 | Hematoma occupying 30% or more of the infarcted |
| | | tissue, with obvious mass effect |
| 3 | | Intracerebral hemorrhage outside the infarcted brain |
| | | tissue or intracranial-extracerebral hemorrhage |
| 3a | | Parenchymal hematoma remote from infarcted brain tissue |
| 3b | | Intraventricular hemorrhage |
| 3c | | Subarachnoid hemorrhage |
| 3d | | Subdural hemorrhage |

HI: hemorrhagic infarction,
PH: parenchymatous hematoma

In one embodiment, said intracranial hemorrhages are from any one of the classes and/or types described hereinabove according to the Heidelberg classification.

In one embodiment, said intracranial hemorrhages are hemorrhagic transformations. In one embodiment, said intracranial hemorrhages are subarachnoid hemorrhages.

Thus, the present invention further relates to an inhibitor, a protein, a composition, a pharmaceutical composition or a medicament of the invention for use in decreasing the incidence of the intracranial hemorrhages or for use in preventing the occurrence of intracranial hemorrhages in a subject affected by, preferably diagnosed with a GPVI-related condition.

In one embodiment, the inhibitor, in particular the protein, for use according to the present invention decreases the risk of death in a subject affected by, preferably diagnosed with a GPVI-related condition. In one embodiment, the risk of death within a time period of 90 days, 80 days, 60 days, 40 days, 20 days, 10 days, or 5 days, after the onset of the GPVI-related condition is decreased. In one embodiment, the risk of death within a time period of 20 days, 10 days, or 5 days, after the onset of the GPVI-related condition is decreased.

In one embodiment, the death may be caused by various events, such as, for example, worsening of the initial stroke, respiratory diseases, ICHs, cerebral herniation, visceral dysfunction, sepsis or hypercapnia. In one embodiment, the death is a non-stroke-related death. In one embodiment, the death is a stroke-related death, such as, for example, worsening of the initial stroke, ICHs or cerebral herniation. In one embodiment, the death is caused by ICHs.

Thus, in one embodiment, the present invention further relates to an inhibitor, a protein, a composition, a pharmaceutical composition or a medicament of the invention for use in decreasing the risk of death in a subject affected by, preferably diagnosed with a GPVI-related condition.

In one embodiment, the present invention further relates to an inhibitor, a protein, a composition, a pharmaceutical composition or a medicament of the invention for use in decreasing the risk of death within a time period of about 90 days, 80 days, 60 days, 40 days, 20 days, 10 days or 5 days, after the onset of the GPVI-related condition in a subject affected by, preferably diagnosed with a GPVI-related condition.

In one embodiment, the present invention further relates to an inhibitor, a protein, a composition, a pharmaceutical composition or a medicament of the invention for use in decreasing the risk of death within a time period of 20 days, 10 days or 5 days, after the onset of the GPVI-related condition in a subject affected by, preferably diagnosed with a GPVI-related condition.

In one embodiment, the inhibitor, in particular the protein, for use according to the present invention increases the survival probability of a subject affected by, preferably diagnosed with a GPVI-related condition. In one embodiment, the survival of probability is increased within a time period of 90 days, 80 days, 60 days, 40 days, 20 days, 10 days, or 5 days, after the onset of the GPVI-related condition is decreased.

Thus, in one embodiment, the present invention further relates to an inhibitor, a protein, a composition, a pharmaceutical composition or a medicament of the invention for use in increasing the survival probability of a subject affected by, preferably diagnosed with a GPVI-related condition.

In one embodiment, the present invention further relates to an inhibitor, a protein, a composition, a pharmaceutical composition or a medicament of the invention for use in increasing the survival probability within a time period of about 90 days, 80 days, 60 days, 40 days, 20 days, 10 days or 5 days, after the onset of the GPVI-related condition in a subject affected by, preferably diagnosed with a GPVI-related condition.

In one embodiment, the inhibitor, in particular the protein, according to the present invention decreases the neurological symptoms of a subject affected with a GPVI-related condition, preferably a stroke, in particular at short term. In one embodiment, said neurological symptoms are evaluated at 6 hours, 12 hours, 24 hours, 36 hours, 48 hours or more after administration of said inhibitor, in particular said protein. In one embodiment, said neurological symptoms are evaluated 6 hours, 12 hours or 24 hours after administration of said inhibitor, in particular said protein.

Means for evaluating neurological symptoms of a subject affected with a GPVI-related condition, in particular a stroke, are well-known by the skilled artisan in the art, and include for example, NIH stroke scale (NIHSS) as described hereinbelow.

Examples of neurological symptoms, include without limitation, alterations of motor abilities, sensory abilities, speech and languages abilities, cognitive abilities and alteration of the level of consciousness.

Thus, in one embodiment, the present invention further relates to an inhibitor, a protein, a composition, a pharmaceutical composition or a medicament of the invention for use in decreasing the neurological symptoms of a subject affected by, preferably diagnosed with a GPVI-related condition, such as a stroke, preferably at short term, more preferably about 6 hours or about 12 hours after the administration of the inhibitor, protein, composition, pharmaceutical composition or medicament of the invention.

Thus, in one embodiment, the present invention further relates to an inhibitor, a protein, a composition, a pharmaceutical composition or a medicament of the invention for use in decreasing the neurological symptoms of a subject affected by, preferably diagnosed with a GPVI-related condition, such as a stroke, preferably at short term, more preferably about 24 hours or about 36 hours or about 48 hours after the administration of the inhibitor, protein, composition, pharmaceutical composition or medicament of the invention.

In one embodiment, the inhibitor, in particular the protein, according to the present invention decreases the degree of disability or dependence of a subject following a GPVI-related condition, preferably a stroke, in particular at long-term. In one embodiment, said disability or dependence is evaluated 90 days after the administration of said inhibitor, in particular said protein.

Thus, in one embodiment, the present invention further relates to an inhibitor, a protein, a composition, a pharmaceutical composition or a medicament of the invention for use in decreasing the degree of disability or dependence of a subject following a GPVI-related condition, such as a stroke, preferably at long-term, more preferably about 30, 60, 90, 120, 150 days or more after the administration of the inhibitor, protein, composition, pharmaceutical composition or medicament of the invention.

In one embodiment, the present invention further relates to an inhibitor, a protein, a composition, a pharmaceutical composition or a medicament of the invention for use in decreasing the degree of disability or dependence of a subject following a GPVI-related condition, such as a stroke, preferably at long-term, more preferably about 90 days after the administration of the inhibitor, protein, composition, pharmaceutical composition or medicament of the invention.

Means for evaluating disability or dependence following a GPVI-related condition, in particular a stroke, are well-known by the skilled artisan in the art, and include for example, the modified Rankin Scale.

The mRS is a commonly used scale for measuring the degree of disability or dependence in the daily activities of people who have suffered a stroke or other causes of neurological disability. The scale runs from 0-6 (see Table 2), running from perfect health without symptoms to death.

TABLE 2

| Score | Symptoms |
|---|---|
| 0 | No symptoms |
| 1 | No significant disability. Able to carry out all usual |
| | activities, despite some symptoms. |
| 2 | Slight disability. Able to look after own affairs without |
| | assistance, but unable to carry out all previous activities. |
| 3 | Moderate disability. Requires some help, but able to walk |
| | unassisted. |
| 4 | Moderately severe disability. Unable to attend to own bodily |
| | needs without assistance, and unable to walk unassisted. |
| 5 | Severe disability. Requires constant nursing care and |
| | attention, bedridden, incontinent. |
| 6 | Dead. |

Thus, in one embodiment, the inhibitor, protein, composition, pharmaceutical composition or medicament of the invention is for use in decreasing the risk of having a modified Rankin Scale (mRS) score equal to or greater than 4 after administration of the inhibitor, protein, composition, pharmaceutical composition or medicament of the invention.

In one embodiment, said score is measured at long term. In one embodiment, said score is measured 30 days, 60 days, 90 days, 120 days, 150 days or more after the administration of the inhibitor, protein, composition, pharmaceutical composition or medicament of the invention. In one embodiment, said score is measured 90 days after the administration of the inhibitor, protein, composition, pharmaceutical composition or medicament of the invention. Thus, in one embodiment, the inhibitor, protein, composition, pharmaceutical composition or medicament of the invention is for use in decreasing the risk of having a modified Rankin Scale (mRS) score equal to or greater than 5, preferably wherein said score is measured 90 days after the administration of the inhibitor, protein, composition, pharmaceutical composition or medicament of the invention.

In one embodiment, the inhibitor, protein, composition, pharmaceutical composition or medicament of the invention is for use in increasing the probability of having a modified Rankin Scale (mRS) score equal to or lower than 3, preferably wherein said score is measured 90 days after the administration of the inhibitor, protein, composition, pharmaceutical composition or medicament of the invention.

In one embodiment, the inhibitor, protein, composition, pharmaceutical composition or medicament of the invention is for use in increasing the probability of having a complete recanalization following thrombectomy (and thrombolysis).

Thus, in one embodiment, the present invention further relates to an inhibitor, a protein, a composition, a pharmaceutical composition or a medicament of the invention for use in increasing the probability of having a complete recanalization following thrombectomy (and thrombolysis).

In one embodiment, the recanalization is evaluated by the modified treatment in cerebral ischemia (mTICI) score. As used herein, the mTICI score is a score graded from 0 to 3 that assess the anterograde reperfusion degree of the middle cerebral artery territory (see Table 3 below).

TABLE 3

| mTICI | |
|---|---|
| Score | Description |
| 0 | No perfusion |
| 1 | antegrade reperfusion past the initial occlusion, but limited |
| | distal branch filling with little or slow distal reperfusion |
| 2a | antegrade reperfusion of less than half of the occluded target |
| | artery previously ischemic territory (e.g. in one major division |
| | of the middle cerebral artery (MCA) |
| 2b | antegrade reperfusion of more than half of the previously |
| | occluded target artery ischemic territory (e.g. in two major |
| | divisions of the MCA and their territories) |
| 2c | near-complete perfusion except for slow flow in a few distal |
| | cortical vessels or presence of small distal cortical emboli |
| 3 | complete antegrade reperfusion of the previously occluded |
| | target artery ischemic territory, with absence of visualised |
| | occlusion in all distal branches |

In one embodiment, the recanalization is complete if the subject presents a score of 3 according to the mTICI score following thrombectomy (and thrombolysis). Thus, in one embodiment, the inhibitor, protein, composition, pharmaceutical composition or medicament of the present invention is for use in increasing the probability of having a mTICI score of 3 following thrombectomy (and thrombolysis).

Thus, in one embodiment, the present invention further relates to an inhibitor, a protein, a composition, a pharmaceutical composition or a medicament of the invention for use in increasing the probability of having a mTICI score of 3 following thrombectomy (and thrombolysis).

In one embodiment, the inhibitor, protein, composition, pharmaceutical composition or medicament of the invention is for use in increasing the probability of having a complete reperfusion following thrombectomy (and thrombolysis).

Thus, in one embodiment, the present invention further relates to an inhibitor, a protein, a composition, a pharmaceutical composition or a medicament of the invention for use in increasing the probability of having a complete reperfusion following thrombectomy (and thrombolysis).

Examples of GPVI-related conditions are well known in the art and include, without limitation, inflammation, thrombosis, disorders associated with abnormal or aberrant megakaryocyte and/or platelet proliferation, differentiation, morphology, migration, aggregation, degranulation and/or function, thrombotic disorders (such as, for example, thrombotic occlusion of coronary arteries), diseases exhibiting quantitative or qualitative platelet dysfunction and diseases displaying endothelial dysfunction, cerebral vascular diseases, coronary diseases, disorders resulting from any blood vessel insult that can result in platelet aggregation, disorders associated with aberrant signal transduction in response to ligands of GPVI, disorders associated with aberrant levels of GPVI expression and/or activity either in cells that normally express GPVI or in cells that do not express GPVI, bone marrow and peripheral blood or disorders in which platelets contribute by modulating inflammatory responses.

In one embodiment, said GPVI-related condition is a cardiovascular disease or event selected from arterial and venous thrombosis, restenosis, acute coronary syndrome, cerebrovascular accidents due to atherosclerosis, myocardial infarction, pulmonary embolism, critical limb ischemia and peripheral artery disease.

In one embodiment, said GPVI-related condition is a venous thrombosis. In one embodiment, said GPVI-related condition is a restenosis. In one embodiment, said GPVI-related condition is an acute coronary syndrome. In one embodiment, said GPVI-related condition is cerebrovascular accidents due to atherosclerosis. In one embodiment, said GPVI-related condition is a myocardial infarction. In one embodiment, said GPVI-related condition is a pulmonary embolism. In one embodiment, said GPVI-related condition is a critical limb ischemia. In one embodiment, said GPVI-related condition is a peripheral artery disease.

Further examples of diseases, disorders or conditions related to GPVI include, but are not limited to, cardiovascular diseases and/or cardiovascular events, such as, for example, arterial thrombosis including atherothrombosis, ischemic events, acute coronary artery syndrome, myocardial infarction (heart attack), acute cerebrovascular ischemia (stroke), percutaneous coronary intervention, stenting thrombosis, ischemic, restenosis, ischemia, (acute and chronic), diseases of the aorta and its branches (such as aortic aneurysm, thrombosis), peripheral artery disease, venous thrombosis, acute phlebitis and pulmonary embolism, cancer-associated thrombosis (Trousseau syndrome), inflammatory thrombosis and thrombosis associated to infection.

In one embodiment, the inhibitor, protein, composition, pharmaceutical composition or medicament of the invention is for treating or for use in treating arterial or venous thrombosis, restenosis, acute coronary syndrome or cerebrovascular accidents due to atherosclerosis, preferably thrombosis.

In one embodiment, the inhibitor, protein, composition, pharmaceutical composition or medicament as described hereinabove is used to modulate leukocyte-platelet in inflammation and/or thrombosis. Therefore, according to an embodiment, the inhibitor, protein, composition, pharmaceutical composition or medicament as described hereinabove is used to treat inflammation and/or thrombosis. In one embodiment, the inhibitor, protein, composition, pharmaceutical composition or medicament as described hereinabove is used to treat thrombo-inflammation.

In one embodiment, the inhibitor, protein, composition, pharmaceutical composition or medicament as described hereinabove is used to modulate, preferably to prevent, platelet adhesion aggregation and degranulation.

In one embodiment, the inhibitor, protein, composition, pharmaceutical composition or medicament as described hereinabove is used to treat thrombotic disorders (such as, for example, thrombotic occlusion of coronary arteries), diseases exhibiting quantitative or qualitative platelet dysfunction and diseases displaying endothelial dysfunction. These diseases include, but are not limited to, coronary artery and cerebral artery diseases.

In one embodiment, the inhibitor, protein, composition, pharmaceutical composition or medicament as described hereinabove is used to treat cerebral vascular diseases or events, including ischemic stroke, venous thromboembolism diseases (such as, for example, diseases involving leg swelling, pain and ulceration, pulmonary embolism, abdominal venous thrombosis), thrombotic microangiopathies, vascular purpura.

In one embodiment, the inhibitor, protein, composition, pharmaceutical composition or medicament as described hereinabove is used to treat stroke, preferably an ischemic stroke.

In one embodiment, said stroke is an acute stroke.

In one embodiment, the GPVI-related disease or condition is an acute ischemic stroke.

In one embodiment, the inhibitor, protein, composition, pharmaceutical composition or medicament as described hereinabove is used to treat coronary diseases (such as, for example, cardiovascular diseases including unstable angina pectoris, myocardial infarction, acute myocardial infarction, coronary artery disease, coronary revascularization, coronary restenosis, ventricular thromboembolism, atherosclerosis, coronary artery disease (e.g., arterial occlusive disorders), plaque formation, cardiac ischemia, including complications related to coronary procedures, such as percutaneous coronary artery angioplasty (balloon angioplasty) procedures). With respect to coronary procedures, such treatment can be achieved via administration of an inhibitor, a protein, a composition, a pharmaceutical composition or a medicament as described above prior to, during, or subsequent to the procedure. In a preferred embodiment, such administration can be utilized to prevent acute cardiac ischemia following angioplasty.

In one embodiment, the inhibitor, protein, composition, pharmaceutical composition or medicament as described hereinabove is used to treat disorders resulting from any blood vessel insult that can result in platelet aggregation. Such blood vessel insults include, but are not limited to, vessel wall injury, such as vessel injuries that result in a highly thrombogenic surface exposed within an otherwise intact blood vessel e.g., vessel wall injuries that result in release of ADP, thrombin and/or epinephrine, fluid shear stress that occurs at the site of vessel narrowing, ruptures and/or tears at the sites of atherosclerotic plaques, and injury resulting from balloon angioplasty or atherectomy.

Further, in certain embodiments, it is preferred that the inhibitor, in particular the protein, of the invention does not affect other platelet attributes or functions, such as agonist-induced platelet shape change (e.g., GPIb-vWF-mediated platelet activation), release of internal platelet granule components, activation of signal transduction pathways or induction of calcium mobilization upon platelet activation by agonists that do not interact with GPVI.

In one embodiment, the inhibitor, protein, composition, pharmaceutical composition or medicament as described hereinabove is used to treat disorders associated with aberrant signal transduction in response to ligands of GPVI (including, without limitation, collagen, fibrin, fibronectin, vitronectin and laminins) or to other extracellular matrix proteins.

In one embodiment, the inhibitor, protein, composition, pharmaceutical composition or medicament as described hereinabove is used to treat disorders associated with aberrant levels of GPVI expression and/or activity either in cells that normally express GPVI or in cells that do not express GPVI. For example, the inhibitor, in particular the protein, of the invention can be used to modulate disorders associated with aberrant expression of GPVI in cancerous (e.g., tumor) cells that do not normally express GPVI. Such disorders can include, for example, ones associated with tumor cell migration and progression to metastasis.

In one embodiment, the inhibitor, protein, composition, pharmaceutical composition or medicament as described hereinabove is used to modulate immunoregulatory functions of platelets.

In one embodiment, the inhibitor, protein, composition, pharmaceutical composition or medicament as described hereinabove is used to treat disorders of bone marrow and peripheral blood.

In one embodiment, the inhibitor, protein, composition, pharmaceutical composition or medicament as described hereinabove is used to treat disorders in which platelets contribute by modulating inflammatory responses including, without limitation, sustained or prolonged inflammation associated with infection, arthritis, fibrosis or disorders in which platelets modulate cell functions including, without limitation, cancer cells proliferation and/or dissemination.

In one embodiment, the subject is a male. In one embodiment, the subject is a female.

In one embodiment, the subject is a child, i.e. younger than 18 years old. In one embodiment, the subject is an adult, i.e. 18 years old or older.

In one embodiment, the subject is of or older than 50, 55, 60, 65, 70, 75, 80, 85, 90, 95 years of age or more. In one embodiment, the subject is of 65 years of age or older. In one embodiment, the subject is of 80 years of age or older.

In one embodiment, the subject is affected by, preferably is diagnosed with a disease, disorder or condition related to GPVI, preferably a cardiovascular disease and/or event.

In one embodiment, the subject is affected by, preferably is diagnosed with a stroke, preferably an ischemic stroke. In one embodiment, the subject is affected, preferably is diagnosed with an acute stroke, more preferably an acute ischemic stroke.

In one embodiment, the subject is affected by, preferably is diagnosed with a moderate GPVI-related condition, such as a moderate form of stroke.

In one embodiment, the subject is affected by, preferably is diagnosed with a severe GPVI-related condition, such as a severe form of stroke.

In one embodiment, the severity of the stroke is assessed by the National Institutes of Health Stroke Scale (NIHSS) score.

The NIHSS is composed of 11 items (see Table 4 below), each of which scores a specific ability between 0-2, 0-3 or 0-4. For each item, a score of 0 typically indicates normal function in that specific ability, while a higher score is indicative of some level of impairment. The individual scores from each item are summed in order to calculate a patient's total NIHSS score. The maximum possible score is 42, with the minimum score being 0.

TABLE 4

| Instructions | Scale definition |
|---|---|
| 1a. Level of Consciousness: | 0 = Alert; keenly responsive. |
| The investigator must choose a response if a full | 1 = Not alert; but arousable by minor |
| evaluation is prevented by such obstacles as an | stimulation to obey, answer, or respond. |
| endotracheal tube, language barrier, orotracheal | 2 = Not alert; requires repeated |
| trauma/bandages. A 3 is scored only if the patient | stimulation to attend, or is obtunded |
| makes no movement (other than reflexive | and requires strong or painful |
| posturing) in response to noxious stimulation | stimulation to make movements (not |
| | stereotyped). |
| | 3 = Responds only with reflex motor or |
| | autonomic effects or totally |
| | unresponsive, flaccid, and areflexic. |
| 1b. LOC Questions: | 0 = Answers both questions correctly. |
| The patient is asked the month and his/her age. | 1 = Answers one question correctly. |
| The answer must be correct - there is no partial | 2 = Answers neither question correctly. |
| credit for being close. Aphasic and stuporous | |
| patients who do not comprehend the questions | |
| will score 2. Patients unable to speak because of | |
| endotracheal intubation, orotracheal trauma, | |
| severe dysarthria from any cause, language | |
| barrier, or any other problem not secondary to | |
| aphasia are given a 1. It is important that only the | |
| initial answer be graded and that the examiner not | |
| “help” the patient with verbal or non-verbal cues. | |
| 1c. LOC Commands: | 0 = Performs both tasks correctly. |
| The patient is asked to open and close the eyes | 1 = Performs one task correctly. |
| and then to grip and release the non-paretic hand. | 2 = Performs neither task correctly. |
| Substitute another one step command if the hands | |
| cannot be used. Credit is given if an unequivocal | |
| attempt is made but not completed due to | |
| weakness. If the patient does not respond to | |
| command, the task should be demonstrated to him | |
| or her (pantomime), and the result scored (i.e., | |
| follows none, one or two commands). Patients | |
| with trauma, amputation, or other physical | |
| impediments should be given suitable one-step | |
| commands. Only the first attempt is scored. | |
| 2. Best Gaze: | 0 = Normal. |
| Only horizontal eye movements will be tested. | 1 = Partial gaze palsy; gaze is |
| Voluntary or reflexive (oculocephalic) eye | abnormal in one or both eyes, but |
| movements will be scored, but caloric testing is | forced deviation or total gaze |
| not done. If the patient has a conjugate deviation | paresis is not present. |
| of the eyes that can be overcome by voluntary or | 2 = Forced deviation, or total gaze |
| reflexive activity, the score will be 1. If a patient | paresis not overcome by the |
| has an isolated peripheral nerve paresis (CN III, | oculocephalic maneuver |
| IV or VI), score a 1. Gaze is testable in all | |
| aphasic patients. Patients with ocular trauma, | |
| bandages, pre-existing blindness, or other | |
| disorder of visual acuity or fields should be | |
| tested with reflexive movements, and a choice | |
| made by the investigator. Establishing eye | |
| contact and then moving about the patient from | |
| side to side will occasionally clarify the | |
| presence of a partial gaze palsy. | |
| 3. Visual: | 0 = No visual loss. |
| Visual fields (upper and lower quadrants) are | 1 = Partial hemianopia. |
| tested by confrontation, using finger counting or | 2 = Complete hemianopia. |
| visual threat, as appropriate. Patients may be | 3 = Bilateral hemianopia (blind |
| encouraged, but if they look at the side of the | including cortical blindness). |
| moving fingers appropriately, this can be scored | |
| as normal. If there is unilateral blindness or | |
| enucleation, visual fields in the remaining eye | |
| are scored. Score 1 only if a clear-cut asymmetry, | |
| including quadrantanopia, is found. If patient | |
| is blind from any cause, score 3. Double | |
| simultaneous stimulation is performed at this | |
| point. If there is extinction, patient receives a 1, | |
| and the results are used to respond to item 11. | |
| 4. Facial Palsy: | 0 = Normal symmetrical movements. |
| Ask - or use pantomime to encourage - the | 1 = Minor paralysis (flattened |
| patient to show teeth or raise eyebrows and close | nasolabial fold, asymmetry on smiling). |
| eyes. Score symmetry of grimace in response to | 2 = Partial paralysis (total or near-total |
| noxious stimuli in the poorly responsive or | paralysis of lower face). |
| non-comprehending patient. If facial | 3 = Complete paralysis of one or both |
| trauma/bandages, orotracheal tube, tape or other | sides (absence of facial movement in |
| physical barriers obscure the face, these should | the upper and lower face). |
| be removed to the extent possible. | |
| 5. Motor Arm: | 0 = No drift; limb holds 90 (or 45) |
| The limb is placed in the appropriate position: | degrees for full 10 seconds. |
| extend the arms (palms down) 90 degrees (if | 1 = Drift; limb holds 90 (or 45) degrees, |
| sitting) or 45 degrees (if supine). Drift is scored | but drifts down before full 10 seconds; |
| if the arm falls before 10 seconds. The aphasic | does not hit bed or other support. |
| patient is encouraged using urgency in the voice | 2 = Some effort against gravity; limb |
| and pantomime, but not noxious stimulation. | cannot get to or maintain (if cued) 90 |
| Each limb is tested in turn, beginning with the | (or 45) degrees, drifts down to bed, but |
| non-paretic arm. Only in the case of amputation | has some effort against gravity. |
| or joint fusion at the shoulder, the examiner | 3 = No effort against gravity; limb falls. |
| should record the score as untestable (UN), and | 4 = No movement. |
| clearly write the explanation for this choice. | UN = Amputation or joint fusion, |
| | explain: |
| | 5a. Left Arm |
| | 5b. Right Arm |
| 6. Motor Leg: | 0 = No drift; leg holds 30-degree |
| The limb is placed in the appropriate position: | position for full 5 seconds. |
| hold the leg at 30 degrees (always tested supine). | 1 = Drift; leg falls by the end of the |
| Drift is scored if the leg falls before 5 seconds. | 5-second period but does not hit bed. |
| The aphasic patient is encouraged using urgency | 2 = Some effort against gravity; leg |
| in the voice and pantomime, but not noxious | falls to bed by 5 seconds, but has |
| stimulation. Each limb is tested in turn, | some effort against gravity. |
| beginning with the non-paretic leg. Only in the | 3 = No effort against gravity; leg |
| case of amputation or joint fusion at the hip, the | falls to bed immediately. |
| examiner should record the score as untestable | 4 = No movement. |
| (UN), and clearly write the explanation for this | UN = Amputation or joint fusion, |
| choice. | explain: |
| | 6a. Left Leg |
| | 6b. Right Leg |
| 7. Limb Ataxia: | 0 = Absent. |
| This item is aimed at finding evidence of a | 1 = Present in one limb. |
| unilateral cerebellar lesion. Test with eyes open. | 2 = Present in two limbs. |
| In case of visual defect, ensure testing is done in | UN = Amputation or joint fusion, |
| intact visual field. The finger-nose-finger and | explain: |
| heel-shin tests are performed on both sides, and | |
| ataxia is scored only if present out of proportion | |
| to weakness. Ataxia is absent in the patient who | |
| cannot understand or is paralyzed. Only in the | |
| case of amputation or joint fusion, the examiner | |
| should record the score as untestable (UN), and | |
| clearly write the explanation for this choice. In | |
| case of blindness, test by having the patient | |
| touch nose from extended arm position. | |
| 8. Sensory: | 0 = Normal; no sensory loss. |
| Sensation or grimace to pinprick when tested, or | 1 = Mild-to-moderate sensory loss; |
| withdrawal from noxious stimulus in the | patient feels pinprick is less sharp or is |
| obtunded or aphasic patient. Only sensory loss | dull on the affected side; or there is a |
| attributed to stroke is scored as abnormal and the | loss of superficial pain with pinprick, |
| examiner should test as many body areas (arms | but patient is aware of being touched. |
| [not hands], legs, trunk, face) as needed to | 2 = Severe to total sensory loss; |
| accurately check for hemisensory loss. A score | patient is not aware of being touched |
| of 2, “severe or total sensory loss,” should only | in the face, arm, and leg. |
| be given when a severe or total loss of sensation | |
| can be clearly demonstrated. Stuporous and | |
| aphasic patients will, therefore, probably score 1 | |
| or 0. The patient with brainstem stroke who has | |
| bilateral loss of sensation is scored 2. If the | |
| patient does not respond and is quadriplegic, | |
| score 2. Patients in a coma (item 1a = 3) are | |
| automatically given a 2 on this item. | |
| 9. Best Language: | 0 = No aphasia; normal. |
| A great deal of information about | 1 = Mild-to-moderate aphasia; some |
| comprehension will be obtained during the | obvious loss of fluency or facility of |
| preceding sections of the examination. For this | comprehension, without significant |
| scale item, the patient is asked to describe what | limitation on ideas expressed or form of |
| is happening in the attached picture, to name the | expression. Reduction of speech and/or |
| items on the attached naming sheet and to read | comprehension, however, makes |
| from the attached list of sentences. | conversation about provided materials |
| Comprehension is judged from responses here, | difficult or impossible. For example, in |
| as well as to all of the commands in the | conversation about provided |
| preceding general neurological exam. If visual | materials, examiner can identify |
| loss interferes with the tests, ask the patient to | picture or naming card content from |
| identify objects placed in the hand, repeat, and | patient's response. |
| produce speech. The intubated patient should be | 2 = Severe aphasia; all communication |
| asked to write. The patient in a coma (item 1a = 3) | is through fragmentary expression; |
| will automatically score 3 on this item. The | great need for inference, questioning, |
| examiner must choose a score for the patient | and guessing by the listener. Range of |
| with stupor or limited cooperation, but a score | information that can be exchanged |
| of 3 should be used only if the patient is mute | is limited; listener carries burden of |
| and follows no one-step commands. | communication. Examiner cannot |
| | identify materials provided from |
| | patient response. |
| | 3 = Mute, global aphasia; no usable |
| | speech or auditory comprehension. |
| 10. Dysarthria: | 0 = Normal. |
| If patient is thought to be normal, an adequate | 1 = Mild-to-moderate dysarthria; |
| sample of speech must be obtained by asking | patient slurs at least some words |
| patient to read or repeat words from the attached | and, at worst, can be understood |
| list. If the patient has severe aphasia, the clarity | with some difficulty. |
| of articulation of spontaneous speech can be | 2 = Severe dysarthria; patient's speech |
| rated. Only if the patient is intubated or has other | is so slurred as to be unintelligible in |
| physical barriers to producing speech, the | the absence of or out of proportion to |
| examiner should record the score as untestable | any dysphasia, or is mute/anarthric. |
| (UN), and clearly write an explanation for this | UN = Intubated or other physical |
| choice. Do not tell the patient why he or she is | barrier, |
| being tested. | explain: |
| 11. Extinction and Inattention (formerly Neglect): | 0 = No abnormality. |
| Sufficient information to identify neglect may | 1 = Visual, tactile, auditory, spatial, |
| be obtained during the prior testing. If the | or personal inattention or extinction |
| patient has a severe visual loss preventing visual | to bilateral simultaneous stimulation |
| double simultaneous stimulation, and the | in one of the sensory modalities. |
| cutaneous stimuli are normal, the score is | 2 = Profound hemi-inattention or |
| normal. If the patient has aphasia but does | extinction to more than one modality; |
| appear to attend to both sides, the score is | does not recognize own hand or orients |
| normal. The presence of visual spatial neglect or | to only one side of space. |
| anosagnosia may also be taken as evidence of | |
| abnormality. Since the abnormality is scored | |
| only if present, the item is never untestable. | |

Then, a score is associated with a stroke severity, as presented in the Table 5 below.

TABLE 5

| Score | Stroke severity |
|---|---|
| 0 | No stroke symptoms |
| 1-4 | Minor stroke |
| 5-15 | Moderate stroke |
| 16-20 | Moderate to severe stroke |
| 21-42 | Severe stroke |

In one embodiment, the subject is affected by, preferably diagnosed with a minor stroke according to the NIHSS score. In one embodiment, the subject is affected by, preferably diagnosed with a moderate stroke according to the NIHSS score. In one embodiment, the subject is affected by, preferably diagnosed with a moderate to severe stroke according to the NIHSS score. In one embodiment, the subject is affected by, preferably diagnosed with a severe stroke according to the NIHSS score.

In one embodiment, the subject is affected by, preferably diagnosed with a moderate to severe stroke.

In one embodiment, the subject is affected by, preferably diagnosed with a moderate stroke according to the NIHSS score, a moderate to severe stroke according to the NIHSS score, or a severe stroke according to the NIHSS score.

In one embodiment, the subject affected by, preferably diagnosed with a stroke has a NIHSS score ranging from 5 to 42 at baseline (i.e. before administration of the inhibitor, in particular the protein, according to the present invention and optionally before the administration of a thrombolytic agent).

In one embodiment, the subject affected by, preferably diagnosed with a stroke has a NIHSS score ranging from 5 to 15 at baseline (i.e. before administration of the inhibitor, in particular the protein, according to the present invention and optionally before the administration of a thrombolytic agent).

In one embodiment, the subject affected by, preferably diagnosed with a stroke has a NIHSS score ranging from 16 to 20 at baseline.

In one embodiment, the subject affected by, preferably diagnosed with a stroke has a NIHSS score ranging from 21 to 42 at baseline.

In one embodiment, the subject affected by, preferably diagnosed with a stroke has a NIHSS score equal or superior to 5 at baseline (i.e. before administration of the inhibitor, in particular the protein, according to the present invention and optionally before the administration of a thrombolytic agent). In one embodiment, the subject affected by, preferably diagnosed with a stroke has a NIHSS score equal or superior to 5, 6, 7, 8, 9, 10, 11, 12, 13, 14, 15 at baseline.

In one embodiment, the subject affected by, preferably diagnosed with a stroke has a NIHSS score equal or superior to 16 at baseline (i.e. before administration of the inhibitor, in particular the protein, according to the present invention and optionally before the administration of a thrombolytic agent). In one embodiment, the subject affected by, preferably diagnosed with a stroke has a NIHSS score equal or superior to 16, 17, 18, 19, 20 at baseline.

In one embodiment, the subject affected by, preferably diagnosed with a stroke has a NIHSS score equal or superior to 21 at baseline (i.e. before administration of the inhibitor, in particular the protein, according to the present invention and optionally before the administration of a thrombolytic agent). In one embodiment, the subject affected by, preferably diagnosed with a stroke has a NIHSS score equal or superior to 21, 22, 23, 24, 25, 26, 27, 28, 29, 30, 31, 32, 33, 34, 35, 36, 37, 38, 39, 40, 41, 42 at baseline.

In one embodiment, the subject affected by, preferably diagnosed with a stroke has a NIHSS score equal or superior to 6, preferably a NIHSS score equal or superior to 10 at baseline (i.e. before administration of the inhibitor, in particular the protein, according to the present invention and optionally before the administration of a thrombolytic agent).

In one embodiment, the subject affected by, preferably diagnosed with a stroke has a NIHSS score equal or inferior to 42 at baseline (i.e. before administration of the inhibitor, in particular the protein, according to the present invention and optionally before the administration of a thrombolytic agent). In one embodiment, the subject affected by, preferably diagnosed with a stroke has a NIHSS score ranging from 5 or 10 to 42 at baseline.

In one embodiment, the subject affected by, preferably diagnosed with a stroke has a NIHSS score equal or inferior to 25 at baseline (i.e. before administration of the inhibitor, in particular the protein, according to the present invention and optionally before the administration of a thrombolytic agent). In one embodiment, the subject affected by, preferably diagnosed with a stroke has a NIHSS score ranging from 5 or 10 to 25 at baseline.

In one embodiment, the subject affected by, preferably diagnosed with a stroke has a NIHSS score equal or inferior to 15 at baseline (i.e. before administration of the inhibitor, in particular the protein, according to the present invention and optionally before the administration of a thrombolytic agent). In one embodiment, the subject affected by, preferably diagnosed with a stroke has a NIHSS score ranging from 5 or 10 to 15 at baseline.

In one embodiment, the subject is undergoing or has undergone thrombectomy. In one embodiment, the subject will undergo thrombectomy.

In one embodiment, the subject is undergoing or has undergone thrombolysis, preferably wherein said thrombolysis is a treatment with a thrombolytic agent as described hereinabove. In one embodiment, the subject will undergo thrombolysis. In one embodiment, the subject has undergone or is undergoing thrombolysis, but is not undergoing or has not undergone thrombectomy. In one embodiment, the subject will undergo thrombolysis, but will not undergo thrombectomy.

In one embodiment, the subject is undergoing or has undergone thrombectomy and thrombolysis, preferably wherein said thrombolysis is a treatment with a thrombolytic agent as described hereinabove. In one embodiment, the subject will undergo thrombectomy and thrombolysis.

In one embodiment, the subject presents at least two or the three following conditions:
(i). said subject is affected with a moderate to severe GPVI-related condition as described hereinabove,
(ii). said subject is of 65 years of age or older, preferably of 80 years of age or older, as described hereinabove, and/or
(iii). said subject is undergoing or has undergone thrombectomy and thrombolysis, as described hereinabove.

In one embodiment, the subject is affected with a moderate to severe GPVI-related condition (preferably a moderate to severe stroke, more preferably the subject presents a NIHSS score at baseline equal or superior to 10) and is of 65 years of age or older (preferably is of 80 years of age or older).

In one embodiment, the subject is affected with a moderate to severe GPVI-related condition (preferably a moderate to severe stroke, more preferably the subject presents a NIHSS score at baseline equal or superior to 10) and is undergoing or has undergone thrombectomy and thrombolysis.

In one embodiment, the subject is 65 years of age or older (preferably is of 80 years of age or older) and is undergoing or has undergone thrombectomy and thrombolysis.

In one embodiment, the subject is 80 years of age or older and is undergoing or has undergone thrombectomy and thrombolysis.

In one embodiment, the subject is affected with a moderate to severe GPVI-related condition (preferably a moderate to severe stroke, more preferably the subject presents a NIHSS score at baseline equal or superior to 10) and is of 65 years of age or older (preferably is of 80 years of age or older) and is undergoing or has undergone thrombectomy and thrombolysis.

In one embodiment, the subject is affected with a moderate to severe GPVI-related condition (preferably a moderate to severe stroke, more preferably the subject presents a NIHSS score at baseline equal or superior to 10) and is of 80 years of age or older and is undergoing or has undergone thrombectomy and thrombolysis.

In one embodiment, the subject experienced a cardiovascular event (such as, for example, thrombosis, stroke, myocardial infarction or a cerebrovascular accident) less than 48 hours, preferably less than 24 hours, 12 hours, 10 hours, 8 hours, 6 hours, 4 hours or less, before the administration of the inhibitor, in particular the protein, for use in the present invention (and optionally before the administration of the thrombolytic agent).

In one embodiment, the inhibitor, protein, composition, pharmaceutical composition or medicament is administered during the first 24 hours, preferably the first 12 hours, 10 hours, 8 hours, 6 hours, 4 hours or less after the onset of the GPVI-related condition.

In one embodiment, the subject receives the inhibitor, protein, composition, pharmaceutical composition or medicament as described hereinabove as part of a treatment protocol.

In one embodiment, said treatment protocol further comprises, before, concomitantly or after the administration of the inhibitor, in particular the protein, of the invention, the administration of another agent useful for treating a GPVI-related condition, such as, for example a thrombolytic agent, preferably t-PA (including native t-PA and recombinant t-PA, as well as modified forms of t-PA that retain the enzymatic and/or fibrinolytic activity of native t-PA).

Thus, in one embodiment, the subject has received, is receiving or will receive a thrombolytic agent, preferably wherein said thrombolytic agent is t-PA (including native t-PA and recombinant t-PA, as well as modified forms of t-PA that retain the enzymatic and/or fibrinolytic activity of native t-PA).

In one embodiment, t-PA is administered during the first 10, 9, 8, 7, 6, 5 hours, preferably the 4.5 hours after the onset of the GPVI-related condition. In one embodiment, t-PA is administered during the first 4.5, 4, 3.5, 3, 2.5, 2, 1.5 or 1 hour(s), preferably the 2 hours after the onset of the GPVI-related condition.

In one embodiment, the subject receives the inhibitor, protein, composition, pharmaceutical composition or medicament as described hereinabove and another agent useful for treating a GPVI-related condition, such as, for example, a thrombolytic agent, preferably t-PA (including native t-PA and recombinant t-PA, as well as modified forms of t-PA that retain the enzymatic and/or fibrinolytic activity of native t-PA) concomitantly.

In one embodiment, the subject receives the inhibitor, protein, composition, pharmaceutical composition or medicament as described hereinabove before the other agent useful for treating a GPVI-related condition. In one embodiment, the inhibitor, protein, composition, pharmaceutical composition or medicament is administered less than 48 hours, 24 hours, 12 hours, 8 hours, 4 hours, 3 hours, 2.5 hours, 2 hours, 1.5 hours, 1 hour or less, before the other agent useful for treating a GPVI-related condition such as, for example, a thrombolytic agent, preferably t-PA (including native t-PA and recombinant t-PA, as well as modified forms of t-PA that retain the enzymatic and/or fibrinolytic activity of native t-PA).

In one embodiment, the subject receives the inhibitor, protein, composition, pharmaceutical composition or medicament as described hereinabove after the other agent useful for treating a GPVI-related condition. In one embodiment, the inhibitor, protein, composition, pharmaceutical composition or medicament is administered less than 48 hours, 24 hours, 12 hours, 8 hours, 4 hours, 3 hours, 2.5 hours, 2 hours, 1.5 hours, 1 hour or less, after the other agent useful for treating a GPVI-related condition such as, for example, a thrombolytic agent, preferably t-PA (including native t-PA and recombinant t-PA, as well as modified forms of t-PA that retain the enzymatic and/or fibrinolytic activity of native t-PA).

In one embodiment, the inhibitor, protein, composition, pharmaceutical composition or medicament is administered less than 2 hours after the other agent useful for treating a GPVI-related condition such as, for example, a thrombolytic agent, preferably t-PA (including native t-PA and recombinant t-PA, as well as modified forms of t-PA that retain the enzymatic and/or fibrinolytic activity of native t-PA).

In one embodiment, said treatment protocol further comprises, before, concomitantly or after the administration of the inhibitor, in particular the protein, of the invention, the treatment of the subject by endovascular treatment, such as, for example, by thrombectomy or by embolectomy. As used herein, the term “thrombectomy” and “mechanical thrombectomy” are equivalent and refer to a mechanical interventional procedure by which a thrombus is removed.

Therefore, in one embodiment, the subject to be treated, was previously treated or is to be treated by endovascular treatment, such as, for example, by thrombectomy. In one embodiment, the subject is undergoing or has undergone thrombectomy.

In one embodiment, the subject receives the inhibitor, protein, composition, pharmaceutical composition or medicament as described hereinabove and an antiplatelet agent.

In one embodiment, said treatment protocol further comprises, before, concomitantly or after the administration of the inhibitor, in particular the protein, of the invention, the administration of an antiplatelet agent.

Thus, in one embodiment, the subject has received, is receiving or will receive an antiplatelet agent.

In one embodiment, the subject receives the inhibitor, protein, composition, pharmaceutical composition or medicament as described hereinabove and an anticoagulant agent.

In one embodiment, said treatment protocol further comprises, before, concomitantly or after the administration of the inhibitor, in particular the protein, of the invention, the administration of an anticoagulant agent.

Thus, in one embodiment, the subject has received, is receiving or will receive an anticoagulant agent.

In one embodiment, the subject receives the inhibitor, protein, composition, pharmaceutical composition or medicament as described hereinabove and at least one other agent, such as, for example, a thrombolytic agent, and/or an antiplatelet agent, and/or an anticoagulant agent.

In one embodiment, the subject has received, is receiving or will receive a thrombolytic agent, and/or an anticoagulant, and/or an antiplatelet agent.

In one embodiment, said treatment protocol further comprises, before, concomitantly or after the administration of the inhibitor, in particular the protein, of the invention, the treatment of the subject by a thrombolytic agent and/or by an antiplatelet agent, and/or by an anticoagulant agent.

In one embodiment, said treatment protocol further comprises, before, concomitantly or after the administration of the inhibitor, in particular the protein, of the invention, the treatment of the subject by a thrombolytic agent and an antiplatelet agent. In one embodiment, said treatment protocol further comprises, before, concomitantly or after the administration of the inhibitor, in particular the protein, of the invention, the treatment of the subject by a thrombolytic agent and an anticoagulant agent. In one embodiment, said treatment protocol further comprises, before, concomitantly or after the administration of the inhibitor, in particular the protein, of the invention, the treatment of the subject by an antiplatelet agent and an anticoagulant agent. In one embodiment, said treatment protocol further comprises, before, concomitantly or after the administration of the inhibitor, in particular the protein, of the invention, the treatment of the subject by a thrombolytic agent, an antiplatelet agent and an anticoagulant agent.

In one embodiment, the thrombolytic agent, the antiplatelet agent and the anticoagulant agent are as described hereinabove.

In one embodiment, said treatment protocol also comprises, before, concomitantly or after the administration of the inhibitor, in particular the protein, of the invention and of at least one of the agents as described hereinabove, the treatment of the subject by an endovascular treatment such as, for example, a thrombectomy or an embolectomy.

Thus, in one embodiment, the subject has received, is receiving or will receive a thrombolytic agent and/or an antiplatelet agent and/or an anticoagulant agent, as described hereinabove, in combination with an endovascular treatment such as, for example, a thrombectomy or an embolectomy.

In one embodiment, said treatment protocol further comprises, before, concomitantly or after the administration of the inhibitor, in particular the protein, of the invention, the treatment of the subject by at least one of the treatments, such as, for example, 1, 2, 3, or all of the treatments, selected from the group comprising or consisting of an endovascular treatment, such as, for example, a thrombectomy or an embolectomy, a thrombolytic agent, an antiplatelet agent and an anticoagulant agent.

Therefore, in one embodiment, the subject to be treated, was previously treated or is to be treated by at least one of the treatments, such as, for example, 1, 2, 3, or all of the treatments, selected from the group comprising or consisting of endovascular treatment, such as, for example, a thrombectomy or an embolectomy, a thrombolytic agent, an antiplatelet agent and an anticoagulant agent.

In one embodiment, a dose of the inhibitor, in particular the protein, for use in the present invention ranging from about 0.5 mg/kg to about 50 mg/kg is administered (or is to be administered) to the patient, preferably ranging from about 1 mg/kg to about 32 mg/kg, more preferably of about 8 mg/kg. In another embodiment, a dose of inhibitor, in particular of humanized protein, ranging from about 2.5 mg/kg to about 25 mg/kg, preferably from about 5 mg/kg to about 15 mg/kg, more preferably of about 8 mg/kg is to be administered to the patient. In one embodiment, a dose of the inhibitor, in particular the protein, for use in the present invention of about 0.5 mg/kg, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or of about 50 mg/kg is administered (or is to be administered) to the subject.

In one embodiment, a dose of the inhibitor, in particular the protein, for use in the present invention ranging from about 30 mg to about 3000 mg is administered (or is to be administered) to the patient, preferably ranging from about 60 mg to about 2000 mg, more preferably of about 100 to about 1000 mg, and even more preferably of about 500 mg. In one embodiment, a dose of the inhibitor, in particular the protein, for use in the present invention of about 30, 60, 62.5, 90, 100, 125, 150, 175, 200, 225, 250, 275, 300, 325, 350, 375, 400, 450, 500, 550, 600, 650, 700, 750, 800, 850, 900, 950, 1000, 1500, 2000, 2500 or of about 3000 mg is administered (or is to be administered) to the subject. In another embodiment, a dose of the inhibitor, in particular the protein, for use in the present invention ranges from about 100 mg to about 2000 mg, from about 125 mg to about 2000 mg, preferably from about 250 mg to about 1000 mg or from about 500 mg to about 1000 mg. In one embodiment, a dose of the inhibitor, in particular the protein, for use in the present invention of about 1000 mg is administered (or is to be administered) to the subject.

The composition will be formulated for administration to the subject. The compositions may be administered parenterally, by inhalation spray, rectally, nasally, or via an implanted reservoir. The term administration used herein includes subcutaneous, intravenous, intramuscular, intra-articular, intra-synovial, intrasternal, intrathecal, intrahepatic, intralesional and intracranial injection or infusion techniques.

In one embodiment, the inhibitor, in particular the protein, for use in the present invention is injected, preferably by intravenous infusion. In another embodiment, the inhibitor, in particular the protein, for use in the present invention is injected intraperitoneally. In another embodiment, the inhibitor, in particular the protein, for use in the present invention is injected intradermally.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Sterile injectable forms of the compositions may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the inhibitor, in particular the protein, for use in the present invention is administered (or is to be administered) to the subject during about 2 hours, 2.5, 3, 3.5, 4, 4.5, 5, 5.5, 6, 6.5, 7, 7.5, 8, 8.5, 9, 9.5, 10, 10.5, 11, 11.5 or during about 12 hours.

In one embodiment, the inhibitor, in particular the protein, for use in the present invention is continuously administered to the subject during at least 2 hours, preferably during at least 4 to 6 hours (e.g. during about 2 hours, 2.5, 3, 3.5, 4, 4.5, 5, 5.5, 6, 6.5, 7, 7.5, 8, 8.5, 9, 9.5, 10, 10.5, 11, 11.5 or during about 12 hours). As used herein, the terms “continuously administered” refers to the administration of a compound for a prolonged period of time with a substantially constant speed of administration.

In another embodiment, a first bolus of the inhibitor, in particular the protein, is injected, followed by the continuous administration of the remaining dose of the inhibitor, in particular the protein.

In one embodiment, said first bolus administration comprises about 10 to 50%, preferably about 20% of the total dosage of the inhibitor, in particular the isolated humanized protein, to be administered. In one embodiment, said first bolus administration comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or about 50% of the total dosage of the inhibitor, in particular the isolated humanized protein, to be administered.

In one embodiment, said first bolus is administered in about 5 to 30 minutes, preferably in about 15 minutes.

In one embodiment, about 20% of the total dosage of the inhibitor, in particular the protein, for use in the present administration are administered during the first 15 minutes of the administration, followed by a continuous administration of the remaining 80% during the next 5 hours 45 minutes.

In one embodiment, said specific administration regimen (continued administration during at least 2 hours, with optionally a first bolus) allows a prolonged effect of the inhibitor, in particular the protein, which may be observed after the end of administration of said inhibitor, in particular said protein, as demonstrated in the Examples. In one embodiment, the effect of the inhibitor, in particular the protein, on platelet aggregation is observed for at least about 1, 2, 3, 4, 5, 6, 12, 18, 24, 36 or 48 hours after the end of the administration of the inhibitor, in particular the protein.

In one embodiment, an inhibitor, in particular a protein, for use in the present invention present in a pharmaceutical composition can be supplied at a concentration ranging from about 1 to about 100 mg/mL, such as, for example, at a concentration of 1 mg/mL, 5 mg/mL, 10 mg/mL, 50 mg/mL or 100 mg/mL. In one embodiment, the inhibitor, in particular the protein, is supplied at a concentration of about 10 mg/mL in either 100 mg (10 mL) or 500 mg (50 mL) single-use vials.

In one embodiment, the pharmaceutical composition may comprise an inhibitor, in particular a protein, of the invention in PBS pH 7.2-7.7.

In one embodiment, the pharmaceutical composition may comprise an inhibitor, in particular a protein, of the invention in sodium citrate buffer 20 mM, NaCl 130 mM, pH 5.0.

Another object of the invention is a method of treating a GPVI-related condition, wherein said method comprises administering to a subject in need thereof an inhibitor of the GPVI signaling pathway as described hereinabove, or a composition, pharmaceutical composition or medicament as described hereinabove, wherein said subject presents at least one of the following conditions:
(i). said subject is affected with a moderate to severe GPVI-related condition,
(ii). said subject is above 65 years old, and/or
(iii). said subject is undergoing or has undergone thrombectomy and thrombolysis,
   preferably wherein said GPVI-related condition is a stroke.

In one embodiment the inhibitor of the GPVI signaling pathway is an isolated protein, preferably an isolated humanized protein, binding to hGPVI.

Another object of the invention is a method of treating a GPVI-related condition, wherein said method comprises administering to a subject in need thereof an isolated humanized protein binding to hGPVI as described hereinabove, or a composition, pharmaceutical composition or medicament as described hereinabove, wherein said subject presents at least one of the following conditions:
(i). said subject is affected with a moderate to severe GPVI-related condition,
(ii). said subject is above 65 years old, and/or
(iii). said subject is undergoing or has undergone thrombectomy and thrombolysis,
   preferably wherein said GPVI-related condition is a stroke.

In one embodiment, the method of the invention comprises administering said inhibitor, protein, composition, pharmaceutical composition or medicament during at least 2 hours to the subject, preferably during at least 4 to 6 hours.

In one embodiment, the method of treating a GPVI-related condition of the present invention further comprises treating the patient with endovascular treatment such as, for example, a thrombectomy. In one embodiment, said endovascular treatment such as, for example, a thrombectomy, is carried out before, concomitantly or after the administration of the inhibitor, in particular the protein (preferably the antibody or fragment thereof), of the present invention.

In one embodiment, the method of treating a GPVI-related condition of the present invention further comprises a step of administering another agent useful for treating a GPVI-related condition, such as, for example a thrombolytic agent, preferably t-PA (including native t-PA and recombinant t-PA, as well as modified forms of t-PA that retain the enzymatic and/or fibrinolytic activity of native t-PA). In one embodiment, said additional agent is administered before, concomitantly or after the administration of the inhibitor, in particular the protein (preferably the antibody or fragment thereof), of the present invention.

In one embodiment, the method of treating a GPVI-related condition of the present invention further comprises a step of administering another agent useful for treating a GPVI-related condition, such as, for example a thrombolytic agent, and/or an anticoagulant, and/or an antiplatelet agent. In one embodiment, the thrombolytic agent, the antiplatelet agent and the anticoagulant agent are as described hereinabove.

In one embodiment, the method of the invention comprises administering a dose of the inhibitor, in particular the protein, as described in the present invention ranging from about 0.5 mg/kg to about 50 mg/kg, preferably ranging from about 1 mg/kg to about 32 mg/kg, more preferably of about 8 mg/kg. In another embodiment, the method of the invention comprises administering a dose of the inhibitor, in particular the protein, as described in the present invention ranging from about 2.5 mg/kg to about 25 mg/kg, preferably from about 5 mg/kg to about 15 mg/kg, more preferably of about 8 mg/kg. In one embodiment, the method of the invention comprises administering a dose of the inhibitor, in particular the protein, as described in the present invention of about 0.5 mg/kg, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or of about 50 mg/kg.

In one embodiment, the method of the invention comprises administering a dose of the inhibitor, in particular the protein, as described in the present invention ranging from about 30 mg to about 3000 mg, preferably ranging from about 60 mg to about 2000 mg, more preferably of about 100 to about 1000 mg, and even more preferably of about 500 mg or of about 1000 mg. In one embodiment, the method of the invention comprises administering a dose of the inhibitor, in particular the protein, as described in the present invention of about 30, 60, 62.5, 90, 100, 125, 150, 175, 200, 225, 250, 275, 300, 325, 350, 375, 400, 450, 500, 550, 600, 650, 700, 750, 800, 850, 900, 950, 1000, 1500, 2000, 2500 or of about 3000 mg. In another embodiment, the method of the invention comprises administering a dose of the inhibitor, in particular the protein, as described in the present invention ranging from about 100 mg to about 2000 mg, from about 125 mg to about 2000 mg, preferably from about 250 mg to about 1000 mg or from about 500 mg to about 1000 mg.

In one embodiment, the inhibitor, in particular the protein, as described in the present invention is injected, preferably by intravenous infusion.

In one embodiment, the method of the invention comprises administering the inhibitor, in particular the protein, during about 2 hours, 2.5, 3, 3.5, 4, 4.5, 5, 5.5, 6, 6.5, 7, 7.5, 8, 8.5, 9, 9.5, 10, 10.5, 11, 11.5 or during about 12 hours.

In one embodiment, the method of the invention comprises continuously administering the inhibitor, in particular the protein, during at least 2 hours, preferably during at least 4 to 6 hours (e.g., during about 2 hours, 2.5, 3, 3.5, 4, 4.5, 5, 5.5, 6, 6.5, 7, 7.5, 8, 8.5, 9, 9.5, 10, 10.5, 11, 11.5 or during about 12 hours).

In another embodiment, the method of the invention comprises administering a first bolus of the inhibitor, in particular the protein, followed by the continuous administration of the remaining dose of the inhibitor, in particular the protein.

In one embodiment, said first bolus administration comprises about 10 to 50%, preferably about 20% of the total dosage of the inhibitor, in particular the isolated humanized protein, to be administered. In one embodiment, said first bolus administration comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or about 50% of the total dosage of the inhibitor, in particular the isolated humanized protein, to be administered.

In one embodiment, said first bolus is administered in about 5 to 30 minutes, preferably in about 15 minutes.

In one embodiment, about 20% of the total dosage of the inhibitor, in particular the protein, for use in the present administration are administered during the first 15 minutes of the administration, followed by a continuous administration of the remaining 80% during the next 5 hours 45 minutes.

In one embodiment, the method of the invention is for inhibiting GPVI receptor function and downstream signalling, thereby treating a GPVI related condition.

In one embodiment, the method for inhibiting GPVI receptor function and downstream signalling does not impact platelet count, expression of GPVI at the platelet surface nor bleeding time.

In one embodiment, the method for inhibiting GPVI receptor function and downstream signalling is efficient and reversible.

In one embodiment, the method of the invention is for inhibiting the binding of GPVI to its ligands (preferably, but not exclusively, collagen), thereby treating a GPVI related condition.

In one embodiment, the method for inhibiting the binding of GPVI to its ligands does not impact platelet count, expression of GPVI at the platelet surface nor bleeding time.

In one embodiment, the method for inhibiting the binding of GPVI to its ligands is efficient and reversible.

In one embodiment, the method of the invention is for inhibiting and/or preventing platelet adhesion to collagen, thereby treating a GPVI related condition.

In one embodiment, the method of the invention is for inhibiting and/or preventing collagen-induced platelet aggregation, thereby treating a GPVI related condition

In one embodiment, the method of the invention is for inhibiting and/or preventing platelet activation, in particular platelet aggregation, in response to collagen, thereby treating a GPVI related condition.

In one embodiment, the method of the invention is for inhibiting and/or preventing thrombin production in response to collagen and/or to tissue factor, thereby treating a GPVI related condition.

In one embodiment, the method of the invention is for inhibiting the binding of GPVI to fibrin, thereby treating a GPVI related condition.

In one embodiment, the method of the invention is for inhibiting and/or preventing platelet recruitment by fibrin via GPVI, thereby treating a GPVI related condition.

In one embodiment, the method of the invention is for inhibiting and/or preventing GPVI-dependent thrombin production in response to fibrin, thereby treating a GPVI related condition.

In one embodiment, the method of the invention is for decreasing the risk of death of the subject. In one embodiment, the risk of death within a time period of 20 days, 10 days, or 5 days, after the onset of the GPVI-related condition is decreased.

Another object of the present invention is a method for decreasing the risk of death of a subject affected, preferably diagnosed with a GPVI-related condition, said method comprising administering an inhibitor, a protein, a composition, a pharmaceutical composition or a medicament to the subject. In one embodiment, the method of the invention is for decreasing the risk of death within a time period of 20 days, 10 days, or 5 days, after the onset of the GPVI-related condition.

Another object of the present invention is a method for increasing the survival probability of a subject affected by, preferably diagnosed with a GPVI-related condition, said method comprising administering an inhibitor, a protein, a composition, a pharmaceutical composition or a medicament to the subject.

In one embodiment, the method of the invention is for preventing the occurrence of intracranial hemorrhages in the subject.

Another object of the present invention is a method for preventing the occurrence of intracranial hemorrhages in a subject affected with a GPVI-related condition, said method comprising administering an inhibitor, a protein, a composition, a pharmaceutical a composition or a medicament to the subject.

In one embodiment, the method of the invention is for decreasing the degree of disability or dependence of a subject following a GPVI-related condition. In one embodiment, the degree of disability or dependence is evaluated 90 days after administration of the inhibitor, in particular the protein, of the present invention.

Another object of the present invention is a method for decreasing the degree of disability or dependence of a subject following a GPVI-related condition, said method comprising administering an inhibitor, a protein, a composition, a pharmaceutical composition or a medicament to the subject, preferably wherein said degree of disability or dependence is evaluated 90 days after administration of the inhibitor, in particular the protein, of the present invention.

Another object of the present invention is a method for decreasing the risk of presenting disability or dependence of a subject following a GPVI-related condition, said method comprising administering an inhibitor, a protein, a composition, a pharmaceutical composition or a medicament to the subject, preferably wherein said degree of disability or dependence is evaluated 90 days after administration of the inhibitor, in particular the protein, of the present invention.

In one embodiment, the method of the invention is for decreasing the risk of having a modified Rankin Scale (mRS) score measured 90 days after the administration of the inhibitor, in particular the isolated humanized protein, equal to or greater than 4.

Another object of the present invention is a method for decreasing the risk of having a modified Rankin Scale (mRS) score equal to or greater than 4, said method comprising administering an inhibitor, a protein, a composition, a pharmaceutical composition or a medicament to subject affected with a GPVI-related condition, preferably wherein said modified Rankin Scale (mRS) score is measured 90 days after administration of the inhibitor, protein, composition, pharmaceutical composition or medicament.

Another object of the present invention is a method for increasing the probability of having a modified Rankin Scale (mRS) score equal to or lower than 3, said method comprising administering an inhibitor, a protein, a composition, a pharmaceutical composition or a medicament to subject affected with a GPVI-related condition, preferably wherein said modified Rankin Scale (mRS) score is measured 90 days after administration of the inhibitor, protein, composition, pharmaceutical composition or medicament.

Another object of the present invention is a method for increasing the probability of having a complete recanalization following thrombectomy (and thrombolysis), said method comprising administering an inhibitor, a protein, composition, pharmaceutical composition or medicament to the subject affected with a GPVI-related condition.

Another object of the present invention is a method for increasing the probability of having a mTICI score of 3 following thrombectomy (and thrombolysis), said method comprising administering an inhibitor, a protein, composition, pharmaceutical composition or medicament to the subject affected with a GPVI-related condition.

Another object of the present invention is a method for increasing the probability of having a complete reperfusion following thrombectomy (and thrombolysis), said method comprising administering an inhibitor, a protein, a composition, a pharmaceutical composition or a medicament to the subject affected with a GPVI-related condition.

In one embodiment, administering an inhibitor, in particular a protein, as described hereinabove to a subject does not induce depletion of GPVI in vivo.

In one embodiment, administering an inhibitor, in particular a protein, as described hereinabove to a subject does not induce a decrease in platelet count. Thus, in one embodiment, administering an inhibitor, a protein, as described hereinabove to a subject does not induce thrombocytopenia.

In one embodiment, administering an inhibitor, in particular a protein, as described hereinabove to a subject does not induce an increase in bleeding time.

In one embodiment, the method of the invention comprises administering a therapeutically effective amount of the inhibitor, in particular the protein, to the subject.

The present invention further relates to a method for enhancing the potency of a thrombolytic agent (preferably t-PA (including native t-PA and recombinant t-PA, as well as modified forms of t-PA that retain the enzymatic and/or fibrinolytic activity of native t-PA)) for treating a GPVI-related condition in a subject in need thereof, wherein said method comprises administering a combination of said thrombolytic agent with an inhibitor, in particular a protein, of the invention (preferably a therapeutically effective amount of the inhibitor, in particular the protein, of the invention) to the subject, wherein said subject presents at least one of the following conditions:
(i). said subject is affected with a moderate to severe GPVI-related condition,
(ii). said subject is above 65 years old, and/or
(iii). said subject is undergoing or has undergone thrombectomy and thrombolysis,
   preferably wherein said GPVI-related disease or condition is a stroke.

In one embodiment, the method of the invention allows decreasing the dose of thrombolytic agent (preferably t-PA) to be administered to the subject.

In one embodiment, the combination of a thrombolytic agent and an inhibitor, in particular a protein, of the invention as described hereinabove is to be administered simultaneously or sequentially. In one embodiment, the thrombolytic agent is to be administered before the inhibitor, in particular the protein, of the invention. In one embodiment, the thrombolytic agent is to be administered after the inhibitor, in particular the protein, of the invention.

### BRIEF DESCRIPTION OF THE DRAWINGS

FIG. 1 is a Kaplan-Meier Curve representing the survival analysis in the group receiving glenzocimab at 1000 mg (continuous line) vs placebo (dashed line) in the phase IIa study.

FIG. 2 is a schema representing the mRS results at Day 90 across treatment groups (glenzocimab at 1000 mg (n=52) vs placebo (n=53)) in the overall population.

FIG. 3 is a schema representing the mRS results at Day 90 across treatment groups (glenzocimab at 1000 mg (n=20) vs placebo (n=23)) in the population of patients above 80 years old.

FIG. 4 is a schema representing the mRS results at Day 90 across treatment groups (glenzocimab at 1000 mg (n=37) vs placebo (n=40)) in the population of patients having a NIHSS score equal or superior to 10 at baseline.

FIG. 5 is a schema representing the mRS results at Day 90 across treatment groups (glenzocimab at 1000 mg (n=28) vs placebo (n=27)) in the population of patients undergoing thrombectomy and thrombolysis.

FIG. 6 is a schema representing the mRS results at Day 90 across treatment groups (glenzocimab at 1000 mg (n=18) vs placebo (n=16)) in the population of patients between 65 and 79 years old.

### EXAMPLES

The present invention is further illustrated by the following examples.

### Example 1: Randomized, Double Blind, Multicenter, Multinational, Placebo-Controlled, Single Parallel Escalating Dose Safety and Efficacy Study of Glenzocimab, Used as Add-on Therapy on Top of the Standard of Care, in the 4.5 Hours Post Onset of Acute Ischemic Stroke Symptoms

The primary objective is to evaluate the safety of single intravenous (bolus+infusion) doses of glenzocimab in add-on to recombinant tPA, with or without added mechanical thrombectomy, monitoring the occurrence of:
Symptomatic and non-symptomatic IntraCranial Hemorrhages (ICHs)
SAEs, SUSARs, Bleeding-related events (BREs), Treatment Emergent Adverse Events (TEAEs), Deaths, medically important events

The secondary objective is to evaluate the following points:
Safety: global profile
Efficacy: early neurological improvement (NIHSS score) and functional handicap recovery (mRS)
PK profile/ADA
Optimal therapeutic dose
Exploratory: Predictive biomarkers or imaging profile and patient characteristics related to therapeutic response.

### Endpoints

The study endpoints for the primary objective (safety) are the following:
Incidence of symptomatic ICHs (defined by an increase of NIHSS score≥4 pts, or ICH-related death)
Incidence of non-symptomatic ICHs (not present at baseline imaging but detected on 24-hr CT scan/MRI)
Incidence, nature and severity of: SAEs, SUSARS, TEAEs, bleeding-related AEs
Deaths (all-cause)

The study endpoints for the secondary objective are the following:
NIHSS score at 24 hrs: absolute and relative change from baseline
Dramatic improvements (defined by a reduction in the NIHSS score of >8 points) or recovery (NIHSS=0) is assessed in each group.
mRS at D90: absolute and relative number of patients with a score 0-2
Recanalization after MT procedure: measured by mTICI score
Size of infarct zone at baseline and D7: assessed by MRI central review
PK: all patients (TO-30 min-24 h) & randomly (5 h-9 hrs or 7.5-12 h)
Safety: Changes to vital signs over the study course duration versus screening; Changes to clinical laboratory assessments (hematology, biochemistry, urinalysis) over the study duration versus screening; ECG over the study duration versus screening;

### Inclusion Criteria

160 evaluable patients were enrolled with the following inclusion criteria:
Adults Male/Female
Patient or legal representative having given a written consent or emergency consent
Patients presenting with an acute disabling ischemic stroke in either the anterior or posterior circulation. The time of onset is known or if unknown, the last time the patient was seen well, was at most 4.5 hours before confirmation of the diagnosis enabling the initiation of alteplase administration within this time-frame.
Patients presenting at least a NIHSS≥6 prior to thrombolysis with tPA;
Treated by thrombolysis by rtPA, and for those eligible, with mechanical thrombectomy
Effective birth control method

### Main Exclusion Criteria

Coma, and/or NIHSS score>25
Patients<18 years of age
Stroke of hemorrhagic origin
Baseline CT-scan evaluation: more than ⅓ of the middle cerebral artery) regions of clear hypodensity on the baseline imaging;
Significant mass effect with midline shift;
Prior ischemic stroke within the past 3 months with sequelae defined by mRS pre stroke≥2.
Contra-indications to thrombolysis with rtPA (after potential remediation)
Patients receiving a dual antiplatelet treatment;
Cardiopulmonary resuscitation within the past 10 days;
Childbirth within the past 10 days,
Epileptic seizure at the onset of symptoms;
Life expectancy<3 months
Pregnant or breastfeeding;
Females of childbearing potential not using effective birth control methods;
Known severe (grade 3 and above) renal impairment or Glomerular Filtration Rate<30 ml/min/1.73 m2 or Serum Creatinine>>2×ULN (1.2 mg/dL for men and 1.0 mg/dL for women) at screening.

### Study Design

Phase Ib: n=60 patients receiving either escalating dose of glenzocimab (ACT017) (125, 250, 500, 1000 mg) or placebo.
Phase IIa: n=106 patients receiving either 1000 mg of glenzocimab or placebo. Glenzocimab and placebo were administered intravenously, with a bolus followed by a continuous infusion (6 hours). Patients were stratified by type of Standard of Care (SOC) administered: Thrombolysis with tPA only; or Thrombolysis with tPA and mechanical thrombectomy.

### Results

### Safety

Regarding the safety of glenzocimab, there were no more frequent bleeding-related events with glenzocimab at 1000 mg compared to placebo. There was no difference between treatment groups regarding reported SUSARs. In addition, no difference in the incidence of SAEs or TEAEs was detected across all treatment groups. Thus, glenzocimab is well-tolerated by the patients.

The incidence of intracranial hemorrhages (ICHs) in phases Ib and IIa was evaluated. Table 6 hereinbelow recapitulates the incidence of ICHs across treatment groups. No symptomatic ICHs were observed in the group receiving glenzocimab at 1000 mg in both phases Ib and IIa. In addition, there was a lower rate of non-symptomatic ICHs in the group receiving glenzocimab at 1000 mg in both phases Ib and IIa. These results show that the administration of glenzocimab decreases the incidence of symptomatic and non-symptomatic ICHs in patients affected with a stroke.

TABLE 6

| Symptomatic ICH incidence | Non-symptomatic ICH incidence |
|---|---|
| 1/102 (1%) glenzocimab all doses | 31/102 (30%) glenzocimab all doses |
| 0/66 (0%) glenzocimab 1000 mg | 20/66 (30%) glenzocimab 1000 mg |
| 5/64 (8%) placebo | 31/64 (48%) placebo |

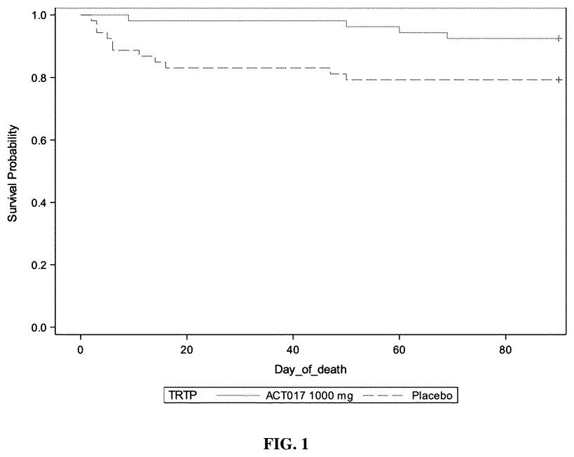

The number of deaths was also evaluated across treatment groups. FIG. 1 shows that there were fewer deaths in the group receiving glenzocimab at 1000 mg compared to placebo. In addition, deaths under placebo occurred earlier than in the group receiving glenzocimab at 1000 mg. There were fewer stroke-related deaths in the group receiving glenzocimab at 1000 mg. Indeed, the number of stroke-related deaths, including initial stroke worsening, ICHs and cerebral herniation, was decreased in the groups receiving glenzocimab as compared to the placebo (4 deaths for glenzocimab at all doses, 3 deaths for glenzocimab at 1000 mg and 8 deaths for placebo). This effect was particularly pronounced for the deaths caused by ICHs: no death caused by ICHs was encountered for glenzocimab at 1000 mg, whereas deaths caused by ICHs were reported in the placebo group (4 deaths caused by ICHs in the placebo group).

Among 106 patients in phase IIa, 43 patients (40.6%) were above 80 years (Min-Max 80-95). Thus, the safety of glenzocimab was also evaluated in this subpopulation. None of said elderly patients have presented s-ICH in the group receiving glenzocimab at 1000 mg against 5 patients in the group receiving the placebo. In overall population, 15 patients died including 7 (47%) elderly patients (5 with placebo, and 2 with glenzocimab) for whom 3 deaths were stroke-related.

Altogether, these results demonstrate that glenzocimab at a dose of 1000 mg showed a very favorable safety profile, particularly in terms of hemorrhagic events and deaths, in the overall population and, in particular, in the subpopulation of patients above 80 years old.

Compatibility with Antiplatelet Agents, Thrombolytic Agents and/or Anticoagulants

Among 106 patients in phase IIa, 27 patients were treated by antiplatelet agents prior to and at study drug administration start (22 aspirin and 5 clopidogrel); and 82 patients were treated by anticoagulants, mostly prophylactic dose of low molecular weight heparins (e.g. Dalteparin or Enoxaparin), or vitamin K antagonists (e.g. acenocoumarol). As shown in Table 7, none of patients treated by antiplatelet agents or by anticoagulants presented s-ICH in the group receiving glenzocimab at 1000 mg against 3 patients treated by antiplatelet agents (20%) and 2 patients treated by anticoagulants (5%) in the group receiving the placebo. The incidence of mortality was not impacted by the association of drugs. No safety signal associated with dual antithrombotic treatment was identified.

TABLE 7

| | | | |
|---|---|---|---|
| | Glenzocimab | | |
| N (%) | 1000 mg | Placebo | Total |

| Patients in | 54 | 52 | | 106 | |
|---|---|---|---|---|---|
| phase 2a (n) | | | | | |
| s-ICH | — | 5 | (10) | 5 | (5) |

| | | | | | | |
|---|---|---|---|---|---|---|
| ns-ICH | 18 | (33) | 26 | (50) | 44 | (42) |
| Death | 4 | (7) | 11 | (21) | 15 | (14) |

Treated by antiplatelet agents (Prior or at study drug
administration start) - n (%)

| | | | | | | |
|---|---|---|---|---|---|---|
| N (%) | 12 | (100) | 15 | (100) | 27 | (100) |

| | | | | | |
|---|---|---|---|---|---|
| s-ICH | — | 3 | (20) | 3 | (11) |

| | | | | | | |
|---|---|---|---|---|---|---|
| ns-ICH | 5 | (42) | 6 | (40) | 11 | (41) |

| | | | | | |
|---|---|---|---|---|---|
| Death | — | 1 | (7) | 1 | (4) |

Treated by anticoagulants (Prior or at study drug
administration start) - n (%)

| | | | | | | |
|---|---|---|---|---|---|---|
| N (%) | 40 | (100) | 42 | (100) | 82 | (100) |

| | | | | | |
|---|---|---|---|---|---|
| s-ICH | — | 2 | (5) | 2 | (2) |

| | | | | | | |
|---|---|---|---|---|---|---|
| ns-ICH | 14 | (35) | 22 | (52) | 36 | (44) |
| Death | 4 | (10) | 6 | (14) | 10 | (8) |

In addition, the safety of glenzocimab in patients with acute ischemic stroke (AIS) exposed to an antithrombotic agent (Alteplase (tPA)) was also evaluated on 54 patients including 11 patients under Acetylsalicylic acid (ASA), regarding the bleeding events. As shown in Table 8, glenzocimab administration was associated with reduced intracranial bleeding rates (33% vs 60% with placebo), even in patients that receive ASA (45% vs 60% with placebo).

TABLE 8

| | | | Alteplase + | Alteplase + |
|---|---|---|---|---|
| | Alteplase + | Alteplase + | Placebo + | ASA + |
| | Placebo | Glenzocimab | ASA | Glenzocimab |
| | (n = 52) | (n = 54) | (n = 10/52) | (n = 11/54) |
| Bleeding | 8 − 7 (13.5) | 10 − 8 (14.8) | 1 − 1 (10) | 4 − 2 (18) |
| events N | | | | |
| events − N | | | | |
| (%) patients | | | | |
| Intracranial | 31 (60) | 18 (33) | 6 (60) | 5 (45) |
| Bleeding N | | | | |
| (%) patients | | | | |

Altogether, these results show that administration of glenzocimab in the subpopulation of patients treated by antiplatelet agents or by anticoagulants has no deleterious impact of the safety for the patients.

These results demonstrate a huge advantage of glenzocimab compared with antiplatelet agents because none of them have shown safety effect associated with anticoagulants. Consequently, these results are in favor of the use of glenzocimab in the subpopulation of patients treated by antiplatelet agents or by anticoagulants.

### Efficacy

Regarding the efficacy, the NIHSS score was evaluated at baseline and at 24 hours across the treatment groups. As seen in the Table 9 hereinbelow, 24 hr NIHSS change from baseline showed no difference between glenzocimab at 1000 mg and placebo on the overall population. However, there was a favorable trend observed in some subpopulations, including i) patients with a NIHSS equal or superior to 10 at baseline, ii) patients undergoing thrombectomy, and iii) patients above 65 years old, and in particular patients above 80 years old.

TABLE 9

| | | | |
|---|---|---|---|
| Phase IIa population | Variable | 1000 mg (n = 53) | Placebo (n = 52) |
| (Absolute changes | Mean (SD) | −3.4 ± 6.3 | −3.4 ± 9.2 |
| from baseline) | | | |
| NIHSS ≥ 10 | Variable | 1000 mg (n = 38) | Placebo (n = 39) |
| (Absolute changes | Mean (SD) | −4.3 ± 7.1 | −3.5 ± 10.5 |
| from baseline) | | | |
| tPA + MT | Variable | 1000 mg (n = 28) | Placebo (n = 26) |
| (Absolute changes | Mean (SD) | −3.2 ± 7.8 | −2.0 ± 11.8 |
| from baseline) | | | |
| Age 65-79 | Variable | 1000 mg (n = 18) | Placebo (n = 16) |
| (Absolute changes | Mean (SD) | −4.1 ± 6.2 | −2.3 ± 12.3 |
| from baseline) | | | |
| Age ≥ 80 | Variable | 1000 mg (n = 20) | Placebo (n = 23) |
| (Absolute changes | Mean (SD) | −4.7 ± 5.3 | −2.6 ± 7.4 |
| from baseline) | | | |

Regarding patients of 80 years or older, 24 hr NIHSS score change from baseline was increased in the group receiving glenzocimab in comparison with the group receiving the placebo (respectively, −4.7±5.3 vs −2.6±7.4) (see Table 10 hereinbelow).

TABLE 10

| | Glenzocimab | |
|---|---|---|
| Patients ≥ 80 years | 1000 mg | Placebo |
| N-n (%) | 20 (47) | 23 (54) |
| Gender | | |
| Female | 13 (65) | 13 (57) |
| Male | 7 (35) | 10 (44) |
| Age | | |
| Mean | 84.5 | 86.0 |
| Median | 83.0 | 85.0 |
| Min-Max | 80-95 | 80-93 |
| NIHSS score ± SD | | |
| Baseline | 15.1 ± 4.5 | 13.3 ± 5.7 |
| 24 hrs | 10.5 ± 6.1 | 10.7 ± 8.8 |
| Δ T0-T24 hrs | −4.7 ± 5.3 | −2.6 ± 7.4 |

Regarding patients with thrombolysis and thrombectomy, 24 hr NIHSS score was improved more frequently in the group receiving glenzocimab in comparison with the group receiving the placebo, and never worsened in the group receiving glenzocimab (see Table 11 hereinbelow). Interestingly, it was noted that thrombectomy was poorly effective in reducing NIHSS score in patients treated with thrombolytic agents and placebo (NIHSS score change from baseline: −0.40), contrary to the condition with glenzocimab, wherein thrombectomy was highly effective in reducing NIHSS score in patients treated with thrombolytic agents and glenzocimab (NIHSS score change from baseline: −5.78).

TABLE 11

| Patients treated by mechanical | Glenzocimab | |
|---|---|---|
| thrombectomy (MT) | 1000 mg | Placebo |
| N | 12 | 8 |
| NIHSS score ± SD | | |
| Improvement from baseline | | |
| At least 4 points | 7 (58) | 2 (25) |
| At least 8 points | 5 (42) | 1 (13) |
| Worsening from baseline | | |
| At least 4 points | 0 | 3 (38) |
| At least 8 points | 0 | 2 (25) |

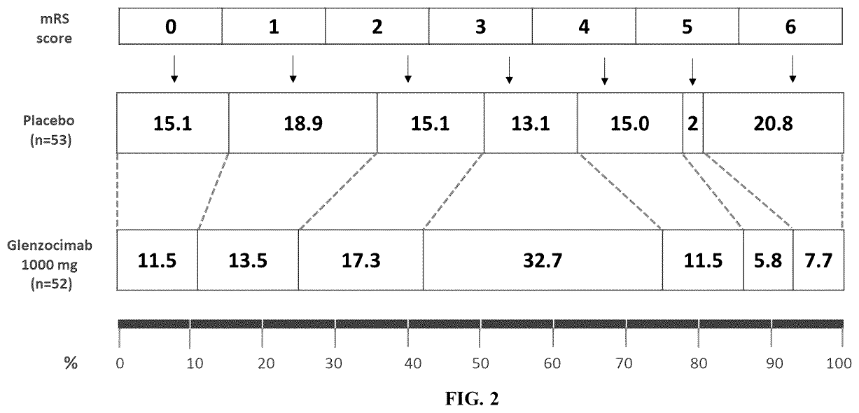

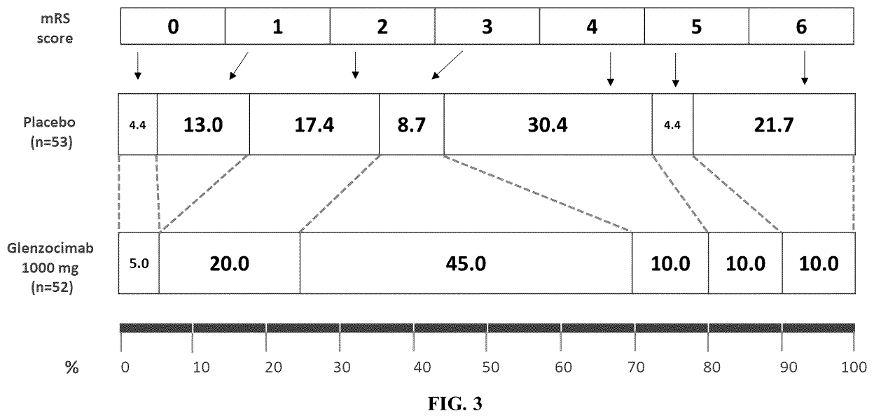

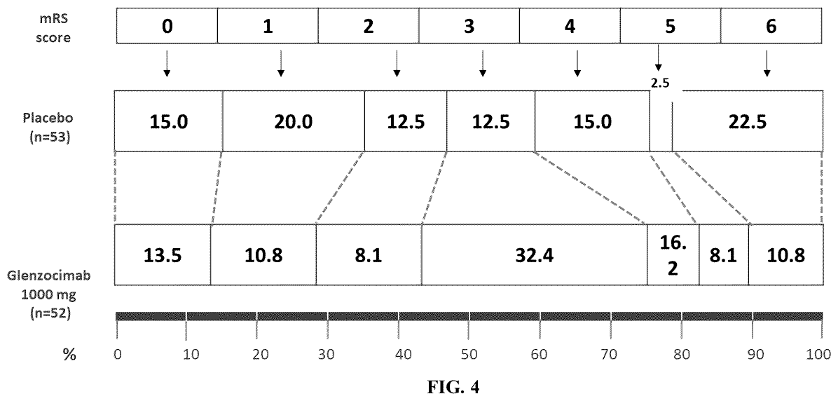

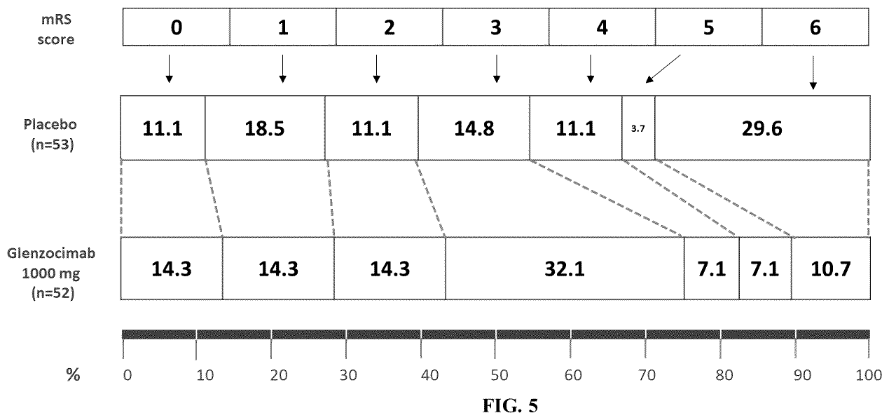

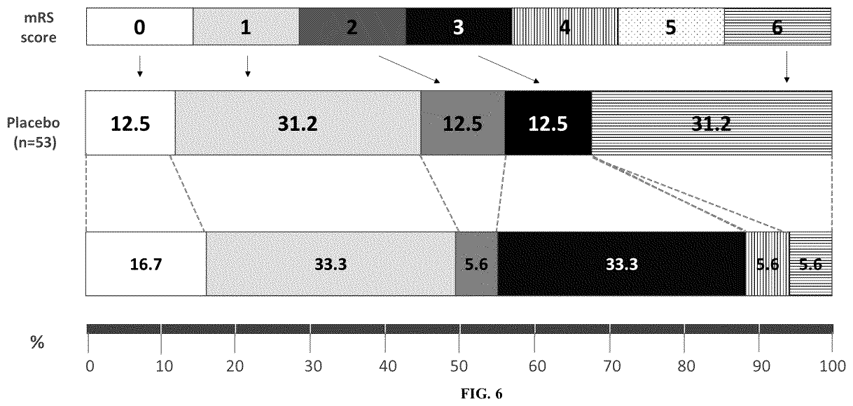

In addition, the mRS score was evaluated at day 90 across treatment groups. As observed in FIG. 2, under glenzocimab treatment at 1000 mg, there was a favorable shift for the most severe mRS categories. Interestingly, this shift was particularly present for patients above 80 years (FIG. 3), patients with a NIHSS score equal or superior to 10 at baseline (FIG. 4), and patients undergoing thrombectomy (FIG. 5). This shift was also present in patients between 65 and 79 years old (FIG. 6).

For the patients having undergone thrombectomy, the recanalization was evaluated after the procedure by measuring the mTICI score across treatment groups. As shown in Table 12, there was a higher rate of success following thrombectomy when the patients were treated with 1000 mg glenzocimab as compared to the patients with placebo. In particular, there is a higher proportion of patients having a mTICI score of 3 (i.e. a complete recanalization) following thrombectomy, when the patients were treated with 1000 mg of glenzocimab as compared to the placebo.

TABLE 12

| | | |
|---|---|---|
| | Glenzocimab | |
| mTICI | 1000 mg | Placebo |
| score | N = 27, n(%) | N = 27, n(%) |

| 0 | 0 | | 3 | (11) |
|---|---|---|---|---|
| 1 | 0 | | 0 | |
| 2a | 1 | (4) | 1 | (4) |
| 2b | 6 | (22) | 5 | (19) |
| 2c | 8 | (30) | 10 | (37) |
| 3 | 12 | (44) | 8 | (30) |
| 2b to 3 | 26 | (96) | 23 | (85) |
| 2c to 3 | 20 | (74) | 18 | (67) |
| Success | 26 | (96) | 23 | (85) |

Regarding the subpopulation of patients above 80 years having mechanical thrombectomy (MT), there was also a higher rate of success following thrombectomy when the patients were treated with 1000 mg glenzocimab as compared to the patients with placebo (see Table 13 hereinbelow). In particular, there is a higher proportion of elderly patients having thrombectomy that have a complete recanalization following thrombectomy, when the patients are treated with 1000 mg of glenzocimab as compared to the placebo (50% vs. 13% with placebo).

TABLE 13

| Patients treated by mechanical | Glenzocimab | |
|---|---|---|
| thrombectomy (MT) | 1000 mg | Placebo |
| N | 12 | 8 |
| Post-MT Recanalization - n (%) | | |
| mTICI score | | |
| Successful Recanalization (grade 2b-3) | 12 (100) | 4 (50) |
| Complete Recanalization (grade 3) | 6 (50) | 1 (13) |

Altogether, these results show that, unexpectedly, administration of glenzocimab in patients suffering from a stroke induces complete recanalizations and reperfusions following thrombectomy, and decreases the incidence of ICHs.

In addition, these results show that there were favorable trends of glenzocimab in patients older than 65 years of age (in particular older than 80 years of age), those undergoing thrombectomy, and those with a moderate to severe stroke. These results also confirm the safety profile of glenzocimab in patients treated with thrombolytic agents, anticoagulants and/or antiplatelet agents.

1-16. (canceled)

17. A method for treating a GPVI-related condition in a subject in need thereof, comprising administering to said subject a therapeutically effective amount of an isolated humanized protein binding to human Glycoprotein VI (hGPVI), wherein said subject presents at least one of the following conditions:
said subject is affected with a moderate to severe GPVI-related condition,
said subject is of 65 years of age or older, and/or
said subject is undergoing or has undergone thrombectomy and thrombolysis,
preferably wherein said GPVI-related condition is a stroke, more preferably an acute ischemic stroke.

18. The method according to claim 17, wherein the isolated humanized protein binding to hGPVI is an antibody molecule or an antibody fragment selected from the group consisting of a whole antibody, a single chain antibody, a Fv, a Fab, a unibody, a domain antibody, and a nanobody; or a monomeric antibody mimetic selected from the group consisting of an affibody, an affilin, an affitin, an adnectin, an atrimer, an evasin, a DARPin, an anticalin, an avimer, a fynomer, and a versabody, preferably the isolated humanized protein is a monovalent antibody or fragment thereof.

19. The method according to claim 17, wherein the isolated humanized protein binding to hGPVI is an antibody molecule or an antibody fragment,
the variable region of the heavy chain comprises at least one of the following CDRs:

| | VH-CDR1: |
|---|---|
| (SEQ ID NO: 1) | |
| | GYTFTSYNMH; |
| | VH-CDR2: |
| (SEQ ID NO: 2) | |
| | GIYPGNGDTSYNQKFQG; |
| | and |
| | VH-CDR3: |
| (SEQ ID NO: 3) | |
| | GTVVGDWYFDV; |

or any CDR having an amino acid sequence that shares at least 60% of identity with SEQ ID NO: 1-3; and
the variable region of the light chain comprises at least one of the following CDRs:

| | VL-CDR1: |
|---|---|
| (SEQ ID NO: 4) | |
| | RSSQSLENSNGNTYLN; |
| | VL-CDR2: |
| (SEQ ID NO: 5) | |
| | RVSNRFS; |
| | and |
| | VL-CDR3: |
| (SEQ ID NO: 6) | |
| | LQLTHVPWT; |

or any CDR having an amino acid sequence that shares at least 60% of identity with SEQ ID NO: 4-6.

20. The method according to claim 17, wherein the isolated humanized protein binding to hGPVI is an antibody molecule or an antibody fragment, the variable region of the heavy chain comprises the following CDRs: GYTFTSYNMH (SEQ ID NO: 1), GIYPGNGDTSYNQKFQG (SEQ ID NO: 2) and GTVVGDWYFDV (SEQ ID NO: 3) and the variable region of the light chain comprises the following CDRs: RSSQSLENSNGNTYLN (SEQ ID NO: 4), RVSNRFS (SEQ ID NO: 5) and LQLTHVPWT (SEQ ID NO: 6) or any CDR having an amino acid sequence that shares at least 60% of identity with said SEQ ID NO: 1-6.

21. The method according to claim 17, wherein the isolated humanized protein binding to hGPVI is an antibody molecule or an antibody fragment, the amino acid sequence encoding the heavy chain variable region is SEQ ID NO: 7 and the amino acid sequence encoding the light variable region is SEQ ID NO: 8, or any sequence having an amino acid sequence that shares at least 60% of identity with said SEQ ID NO: 7 or 8.

22. The method according to claim 17, wherein the isolated humanized protein binding to hGPVI is an antibody molecule or an antibody fragment, the amino acid sequence encoding the heavy chain variable region is SEQ ID NO: 7 and the amino acid sequence encoding the light variable region is SEQ ID NO: 9, or any sequence having an amino acid sequence that shares at least 60% of identity with said SEQ ID NO: 7 or 9.

23. The method according to claim 17, wherein the GPVI-related condition is a stroke, and wherein the severity of the stroke is evaluated by the NIH stroke scale (NIHSS), preferably wherein said subject affected with a stroke has a NIHSS score at baseline equal to or greater than 10.

24. The method according to claim 17, wherein said GPVI-related condition is an ischemic stroke, preferably an acute ischemic stroke.

25. The method according to claim 17, wherein said subject has received, is receiving or will receive a thrombolytic agent, and/or an anticoagulant, and/or an antiplatelet agent.

26. The method according to claim 17, wherein said subject is of 80 years of age or older.

27. The method according to claim 17, wherein said protein prevents the occurrence of intracranial hemorrhages in the subject.

28. The method according to claim 17, wherein said protein decreases the risk of death of the subject, preferably the risk of death within a time period of 20 days after the onset of the GPVI-related condition, more preferably 10 days after the onset of the GPVI-related condition, and even more preferably the risk of death within a time period of 5 days after the onset of the GPVI-related condition.

29. The method according to claim 17, wherein said protein decreases the degree of disability or dependence following the GPVI-related condition.

30. A method for decreasing the degree of disability or dependence following a GPVI-related condition, such as a stroke, in a subject, comprising administering to said subject a therapeutically effective amount of an isolated humanized protein binding to human Glycoprotein VI (hGPVI), preferably the method decreasing the risk of having a modified Rankin Scale (mRS) score measured 90 days after the administration of the isolated humanized protein equal to or greater than 4 or increasing the probability of having a mRS score measured 90 days after the administration of the isolated humanized protein equal to or lower than 3.

31. A method for preventing the occurrence of intracranial hemorrhages in a subject affected with a GPVI-related condition, comprising administering to said subject a therapeutically effective amount of an isolated humanized protein binding to human Glycoprotein VI (hGPVI), wherein preferably said GPVI-related condition is a stroke, more preferably an acute ischemic stroke.

32. A method for decreasing the risk of death in a subject affected with a GPVI-related condition, preferably for decreasing the risk of death within a time period of 20 days after the onset of the GPVI-related condition, preferably within a time period of 10 days after the onset of the GPVI-related condition, and even more preferably the risk of death within a time period of 5 days after the onset of the GPVI-related condition, the method comprising administering to said subject a therapeutically effective amount of an isolated humanized protein binding to human Glycoprotein VI (hGPVI), wherein preferably said GPVI-related condition is a stroke, more preferably an acute ischemic stroke.
